(19) United States
(12) Patent Application Publication
(10) Pub. No.: US 2024/0166680 A1
(43) Pub. Date: May 23, 2024

(54) NUCLEOSIDE ANALOG AND USE THEREOF

(71) Applicant: SHANGHAI INSTITUTE OF MATERIA MEDICA, CHINESE ACADEMY OF SCIENCES, Shanghai (CN)

(72) Inventors: Jingshan Shen, Shanghai (CN); Yuanchao Xie, Shanghai (CN); Tianwen Hu, Shanghai (CN)

(21) Appl. No.: 18/286,416

(22) PCT Filed: Apr. 12, 2022
(86) PCT No.: PCT/CN2022/086215

§ 371 Date: Oct. 11, 2023

(30) Foreign Application Priority Data
Apr. 15, 2021 (CN) 202110405657.6
Sep. 2, 2021 (CN) 202111027057.7

(51) Int. Cl. C07H 19/23; A61P 31/14; A61P 31/16; C07H 19/067; C07H 19/10

(52) U.S. Cl. CPC C07H 19/23; A61P 31/14; A61P 31/16; C07H 19/067; C07H 19/10

(57) ABSTRACT

The present invention relates to a nucleoside analog represented by the following formula and a use thereof. Specifically, the present invention relates to a nucleoside analog represented by the following formula or a pharmaceutically acceptable salt thereof, and a pharmaceutical composition thereof, and a use thereof in preparation of (a) an inhibitor for inhibiting replication of coronaviruses, paramyxoviruses, influenza viruses, flaviviruses, filoviruses, bunya viruses and/or arenaviruses, and/or (b) a medicine for treating and/or preventing or alleviating a disease caused by infection of coronaviruses, paramyxoviruses, influenza viruses, flaviviruses, filoviruses, bunya viruses and/or arenaviruses.

### TECHNICAL FIELD

The present invention relates to a nucleoside analog and use thereof, specifically, to a compound of formula (I) or a pharmaceutically acceptable salt thereof, a pharmaceutical composition and use thereof.

### BACKGROUND ART

Infectious diseases caused by viruses pose a major threat to global public health security. Since 2007, the World Health Organization has declared a number of global public health emergencies, such as the H1N1 influenza A virus outbreak in 2009, the wild-type poliovirus outbreak in 2014, the Ebola virus outbreak in West Africa in 2014, the Zika virus outbreak in Brazil in 2016, the Ebola outbreak in Congo (Kinshasa) in 2018, and the novel coronavirus (SARS-CoV-2) outbreak in 2020.

There are many types of viruses. In addition to the above, many other viruses have also had a great impact on human society, such as dengue virus, respiratory syncytial virus, bunya virus, and animal coronaviruses that cause heavy losses in the breeding industry. With the expansion of the scope of human social activities and the enhancement of the trend of globalization, new viruses or re-emerging viruses will continue to appear around the world, and the world's medical and health systems will face more severe challenges.

Nucleoside analogs are the most important class of antiviral drugs. This type of drug can be converted into the corresponding triphosphate form in vivo. During the virus replication stage, the triphosphate can “disguise” as a substrate and be incorporated into the DNA or RNA chain of the virus under the catalysis of the viral polymerase, interfering with the replication of genetic material, thereby exerting an antiviral effect. Most viral polymerases have conserved active centers, so nucleoside analogs, as antiviral drugs, have a high barrier to drug resistance and often exhibit broad-spectrum antiviral effects.

β-d-N4-hydroxycytidine (NHC) is a cytosine nucleoside derivative, which was first reported in 1959. The compound exhibits significant inhibitory effect on the replication of various viruses, such as influenza virus, hepatitis C virus, SARS, MERS, SARS-CoV-2, etc., and is a broad-spectrum antiviral nucleoside analogue. (2R,3R,4S,5R)-2-(4-aminopyrrolo[2,1-F][1,2,4]triazin-7-yl)-3,4-dihydroxy-5-(hydroxymethyl)tetrahydrofuran-2-carbonitrile (GS-441524) is also a nucleoside with broad-spectrum antiviral activity. The compound was first discovered to have an anti-hepatitis C virus effect, and was subsequently found to have inhibitory activity against filoviruses (Ebola virus, Marburg virus), paramyxoviruses (paraflu virus, measles virus, respiratory syncytial virus), coronavirus (SARS, MERS, SARS-CoV-2), etc. However, both compounds have the disadvantage of low oral bioavailability, which is less than 10% in monkeys, making them difficult to develop as oral drugs.

Prodrug modification is an important means to improve the drugability of nucleoside analogs. Appropriate prodrug forms are not only beneficial to improve the metabolic properties of such compounds, but also improve their therapeutic effect on diseases and reduce side effects.

### SUMMARY OF THE INVENTION

Therefore, the purpose of the present invention is to provide an active ingredient that can effectively inhibit virus replication, and its new use in related diseases caused by viral infection.

Specifically, the present invention provides a nucleoside analog represented by formula I or pharmaceutically acceptable salts thereof, compositions thereof and use thereof against virus, such as coronaviruses (SARS, MERS, SARS-CoV-2, porcine epidemic diarrhea virus, feline infectious peritonitis virus, etc.), paramyxovirus (paraflu virus, measles virus, respiratory syncytial virus, etc.), influenza virus, flaviviridae virus (hepatitis C virus, dengue virus, Zika virus, etc.), filoviruses (Ebola virus, Marburg virus), bunya virus and/or arenavirus, especially the novel coronavirus (SARS-CoV-2), influenza virus.

According to a first aspect of the present invention, provided is a compound represented by formula I or a pharmaceutically acceptable salt thereof:

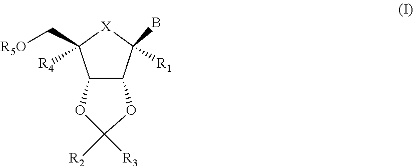

wherein
B is selected from the group consisting of

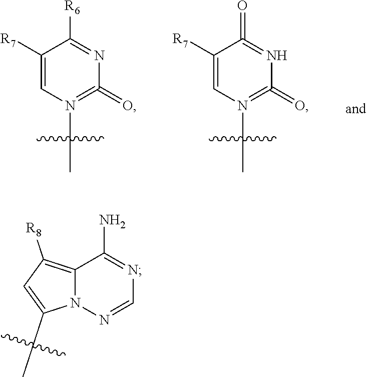

X is selected from the group consisting of oxygen, sulfur, CH_{2,} and NH;
R₁ is selected from the group consisting of hydrogen, deuterium, and cyano;
R₂ is selected from the group consisting of hydrogen, C₁₋₁₈ alkyl, C₃₋₈ cycloalkyl, C₆₋₂₀ aryl, and 5-15 membered heteroaryl, wherein the alkyl and the cycloalkyl are unsubstituted or substituted by one to three substituents independently selected from the group consisting of halogen, hydroxyl, carboxyl and C₁₋₄ alkoxy, and the aryl and the heteroaryl are unsubstituted or substituted by one to five substituents independently selected from the group consisting of R₉;
R₃ is selected from the group consisting of hydrogen, and C₁₋₄ alkoxy;

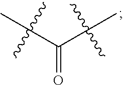 [SMILES: *C(*)=O]

or R₂, R₃ and the carbon to which they are attached form
R₄ is selected from the group consisting of hydrogen, deuterium, halogen, azido, cyano, C₁₋₆ alkyl, halogenated C₁₋₆ alkyl, azido C₁₋₆ alkyl, cyano C₁₋₆ alkyl, hydroxy C₁-6 alkyl, C₂₋₆ alkenyl, C₂₋₆ alkynyl, C₃₋₆ cycloalkyl, and C₁₋₆ alkoxy C₁₋₆ alkyl;
R₅ is selected from the group consisting of hydrogen, C₁₋₂₀ alkanoyl, C₃₋₂₀ cycloalkanoyl, amino C₁₋₂₀ alkanoyl, C₁₋₂₀ alkylamino C₁₋₆ alkanoyl, C₁₋₆ cycloalkylamino C₁₋₆ alkanoyl, C₁₋₂₀ bis-alkylamino C₁₋₆ alkanoyl, C₁₋₂₀ alkoxy C₁₋₆ alkanoyl, an amino acid group in which the carbonyl of the carboxyl group on the amino acid forms an ester bond with the connected oxygen, C₆₋₂₀ arylamino C₁₋₆ alkanoyl, 3-20 membered heterocycloalkyl C₁₋₆ alkanoyl,

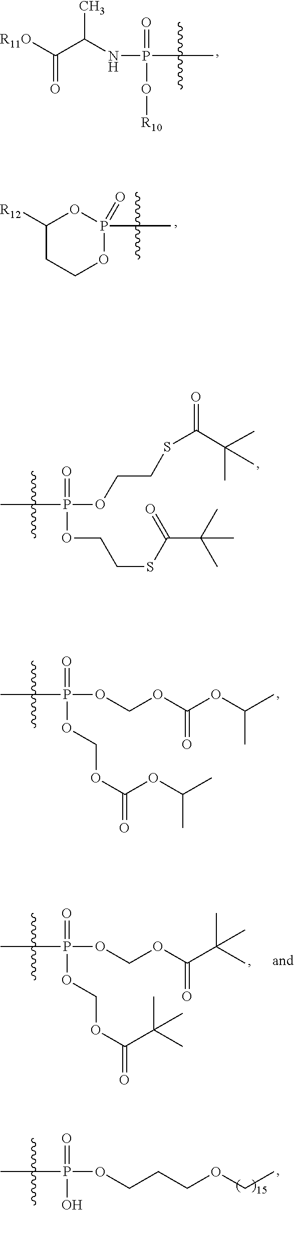

wherein the C₁₋₂₀ alkanoyl and the C₃₋₂₀ cycloalkanoyl are unsubstituted or substituted by one to three halogens, and the 3-20 membered heterocycloalkyl is unsubstituted or substituted by C₁₋₆ alkyl;
R₆ is selected from the group consisting of hydroxyl, amino, hydroxylamine (—NHOH), and —NHOR₁₃;
R₇ is selected from the group consisting of hydrogen, deuterium, and halogen;
R₈ is selected from the group consisting of hydrogen, deuterium, halogen, cyano, and carbamoyl;
R₉ is selected from the group consisting of halogen, C₁₋₄ alkyl, C₁₋₄ alkoxy, C₁₋₄ alkylthio, cyano, nitro, amino, phenyl, carboxyl, trifluoromethyl, difluoromethoxy, trifluoromethoxy, C₁₋₄ alkylamino, di(C₁₋₄ alkyl) amino, C₁₋₄ alkylcarbonyl, C₁₋₄ alkylcarbonyloxy, and C₁₋₄ alkoxycarbonyl;
R₁₀ is selected from the group consisting of C₁₋₆ alkyl, C₃₋₆ cycloalkyl, C₆₋₂₀ aryl, and 5-15 membered heteroaryl;
R₁₁ is selected from the group consisting of C₁₋₁₈ alkyl, and methylene C₆₋₂₀ aryl;
R₁₂ is selected from the group consisting of C₁₋₆ alkyl, C₃₋₆ cycloalkyl, C₆₋₂₀ aryl, and 5-15 membered heteroaryl;
R₁₃ is selected from the group consisting of C₁₋₂₀ alkanoyl, C₃₋₂₀ cycloalkanoyl, amino C₁₋₂₀ alkanoyl, C₁₋₂₀ alkylamino C₁₋₆ alkanoyl, C₁₋₆ cycloalkylamino C₁₋₆ alkanoyl, C₁₋₂₀ dialkylamino C₁₋₆ alkanoyl, C₁₋₂₀ alkoxy C₁₋₆ alkanoyl, and C₁₋₆ alkoxycarbonyloxymethylene;

In a preferred embodiment, the compound of formula I has formula I-I,

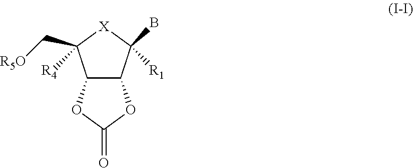

wherein, B, X, R₁, R₄, and R₅ are defined the same as above.

In another preferred embodiment, the compound of formula I has formula I-II,

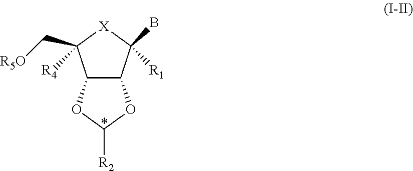

wherein the formula (I-II) contains an asymmetric center indicated by *, so it can have two diastereomers as shown by formulae I-IIA and I-IIB, and thus it may be a pure single diastereomer alone, or a mixture of the two diastereomers,

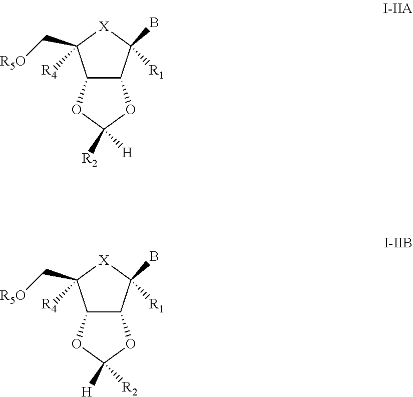

wherein, B, X, R₁, R₂, R₄ and R₅ are defined the same as above.

In another preferred embodiment, the compound of formula I is selected from the group consisting of the following formula,

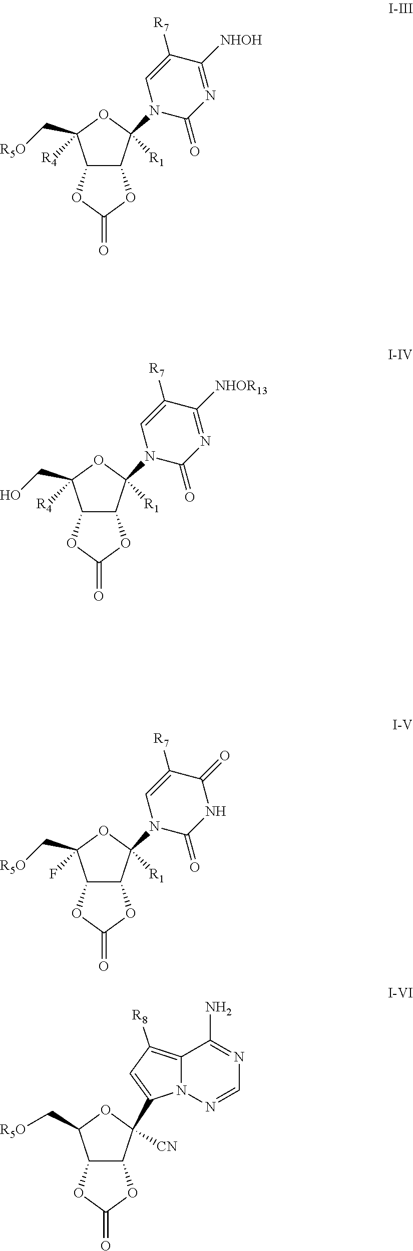

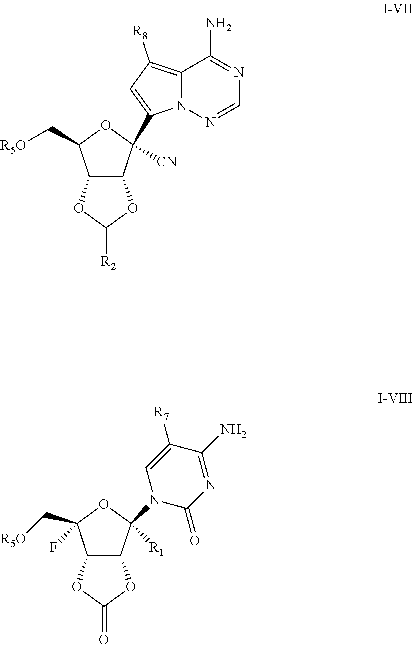

wherein R₁ is selected from the group consisting of hydrogen, and deuterium; R₂, R₅, R₇, R₈ and R₁₃ are defined the same as above;
R₄ is selected from the group consisting of hydrogen, deuterium, and halogen.

In another preferred embodiment, the compound of formula I is any one selected from the group consisting of the compounds A1 to A56, B1 to B46, C1 to C42, and D1 to D13, or a combination thereof:

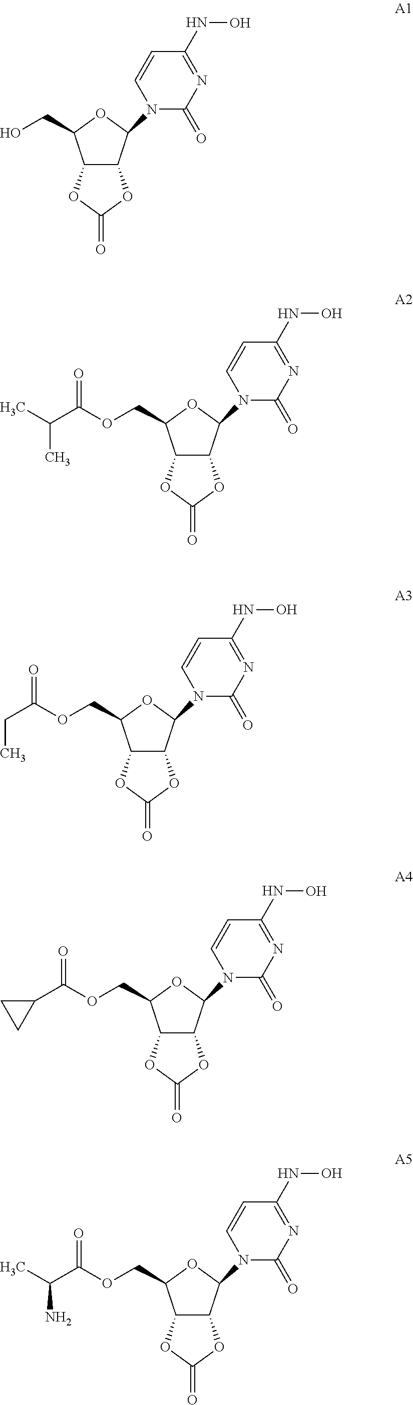 [SMILES: O=C1O[C@@H]2[C@H](O1)[C@@H](CO)O[C@H]2n1ccc(NO)nc1=O ; CC(C)C(=O)OC[C@H]1O[C@@H](n2ccc(NO)nc2=O)[C@@H]2OC(=O)O[C@@H]21 ; CCC(=O)OC[C@H]1O[C@@H](n2ccc(NO)nc2=O)[C@@H]2OC(=O)O[C@@H]21 ; O=C1O[C@@H]2[C@H](O1)[C@@H](COC(=O)C1CC1)O[C@H]2n1ccc(NO)nc1=O ; C[C@H](N)C(=O)OC[C@H]1O[C@@H](n2ccc(NO)nc2=O)[C@@H]2OC(=O)O[C@@H]21]

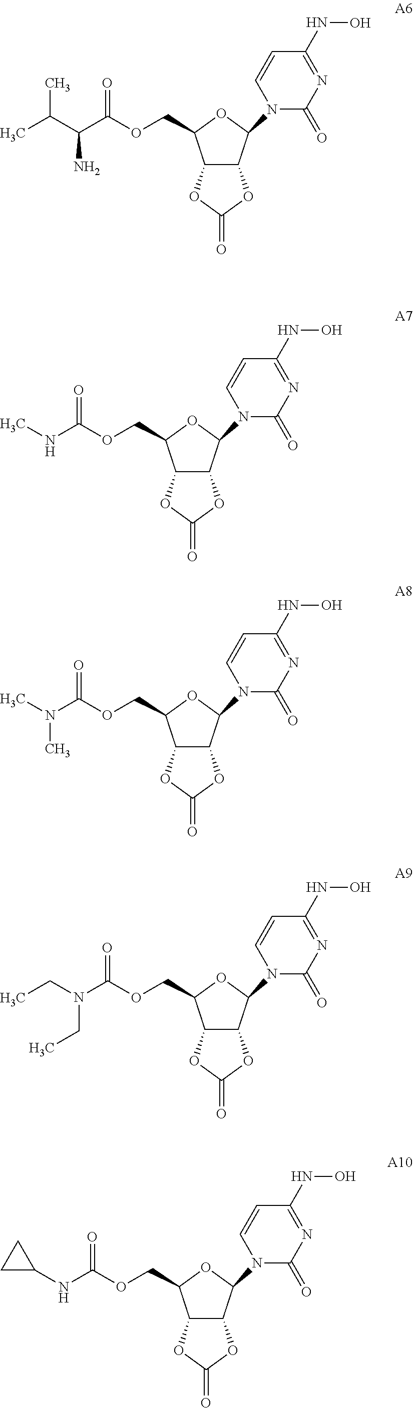 [SMILES: CC(C)[C@H](N)C(=O)OC[C@H]1O[C@@H](n2ccc(NO)nc2=O)[C@@H]2OC(=O)O[C@@H]21 ; CNC(=O)OC[C@H]1O[C@@H](n2ccc(NO)nc2=O)[C@@H]2OC(=O)O[C@@H]21 ; CN(C)C(=O)OC[C@H]1O[C@@H](n2ccc(NO)nc2=O)[C@@H]2OC(=O)O[C@@H]21 ; CCN(CC)C(=O)OC[C@H]1O[C@@H](n2ccc(NO)nc2=O)[C@@H]2OC(=O)O[C@@H]21 ; O=C(NC1CC1)OC[C@H]1O[C@@H](n2ccc(NO)nc2=O)[C@@H]2OC(=O)O[C@@H]21]

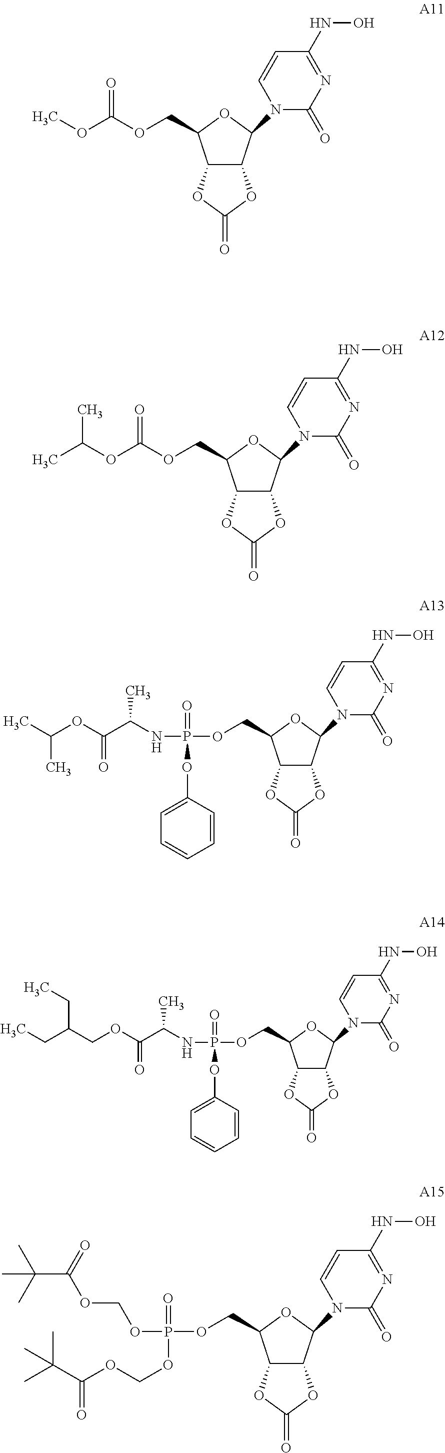 [SMILES: COC(=O)OC[C@H]1O[C@@H](n2ccc(NO)nc2=O)[C@@H]2OC(=O)O[C@@H]21 ; CC(C)OC(=O)OC[C@H]1O[C@@H](n2ccc(NO)nc2=O)[C@@H]2OC(=O)O[C@@H]21 ; CC(C)OC(=O)[C@H](C)N[P@](=O)(OC[C@H]1O[C@@H](n2ccc(NO)nc2=O)[C@@H]2OC(=O)O[C@@H]21)Oc1ccccc1 ; CCC(CC)COC(=O)[C@H](C)N[P@](=O)(OC[C@H]1O[C@@H](n2ccc(NO)nc2=O)[C@@H]2OC(=O)O[C@@H]21)Oc1ccccc1 ; CC(C)(C)C(=O)OCOP(=O)(OCOC(=O)C(C)(C)C)OC[C@H]1O[C@@H](n2ccc(NO)nc2=O)[C@@H]2OC(=O)O[C@@H]21]

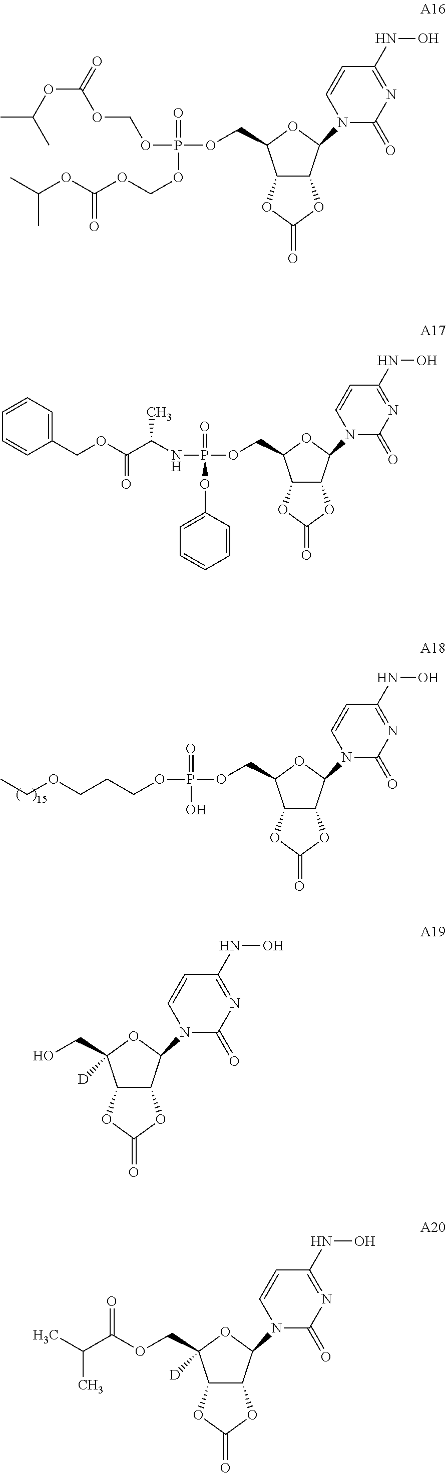

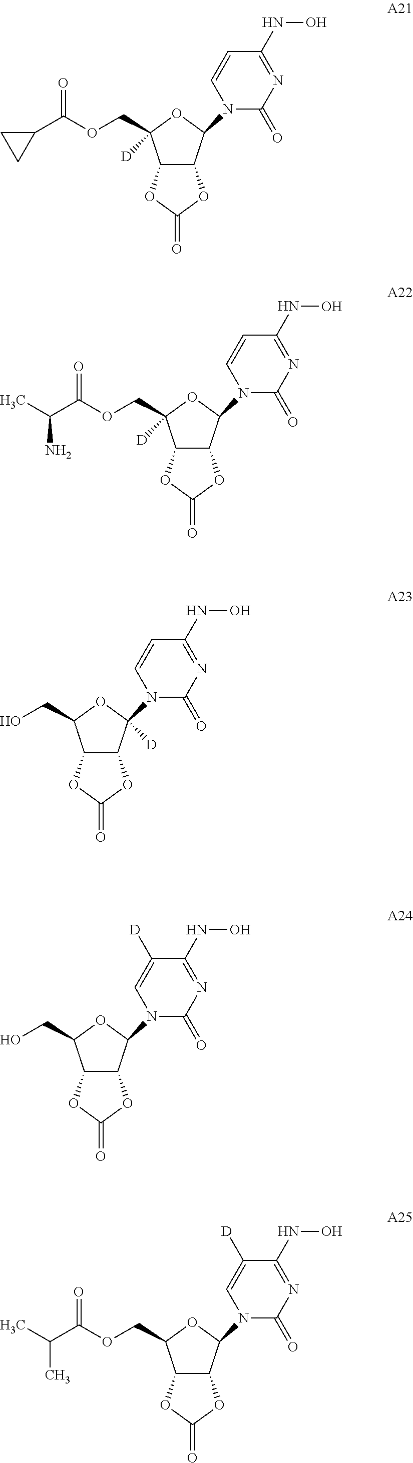 [SMILES: [2H][C@]1(COC(=O)C2CC2)O[C@@H](n2ccc(NO)nc2=O)[C@@H]2OC(=O)O[C@@H]21 ; [2H][C@]1(COC(=O)[C@H](C)N)O[C@@H](n2ccc(NO)nc2=O)[C@@H]2OC(=O)O[C@@H]21 ; [2H][C@@]1(n2ccc(NO)nc2=O)O[C@H](CO)[C@H]2OC(=O)O[C@H]21 ; [2H]c1cn([C@@H]2O[C@H](CO)[C@H]3OC(=O)O[C@H]32)c(=O)nc1NO ; [2H]c1cn([C@@H]2O[C@H](COC(=O)C(C)C)[C@H]3OC(=O)O[C@H]32)c(=O)nc1NO]

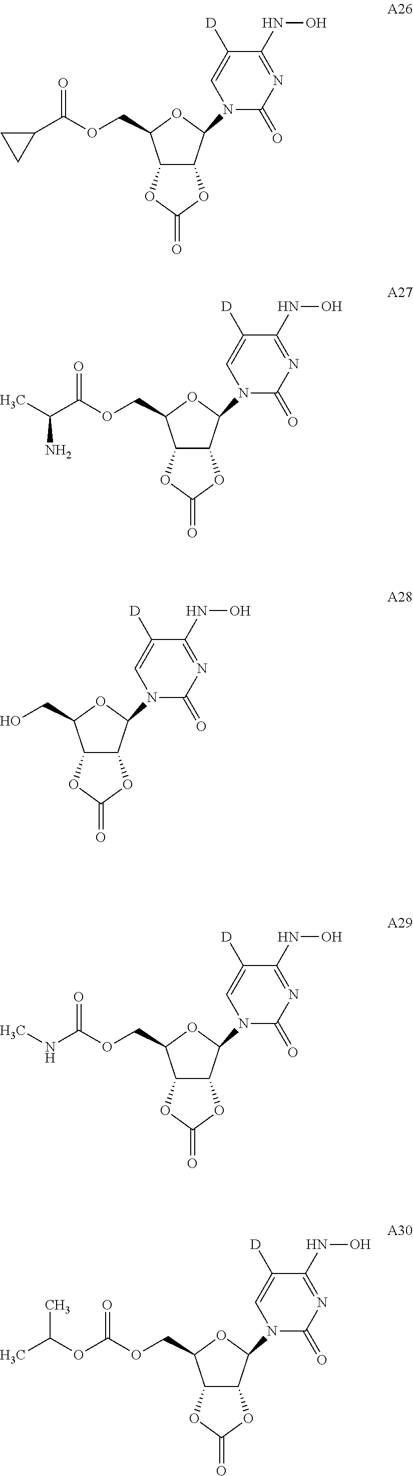 [SMILES: [2H]c1cn([C@@H]2O[C@H](COC(=O)C3CC3)[C@H]3OC(=O)O[C@H]32)c(=O)nc1NO ; [2H]c1cn([C@@H]2O[C@H](COC(=O)[C@H](C)N)[C@H]3OC(=O)O[C@H]32)c(=O)nc1NO ; [2H]c1cn([C@@H]2O[C@H](CO)[C@H]3OC(=O)O[C@H]32)c(=O)nc1NO ; [2H]c1cn([C@@H]2O[C@H](COC(=O)NC)[C@H]3OC(=O)O[C@H]32)c(=O)nc1NO ; [2H]c1cn([C@@H]2O[C@H](COC(=O)OC(C)C)[C@H]3OC(=O)O[C@H]32)c(=O)nc1NO]

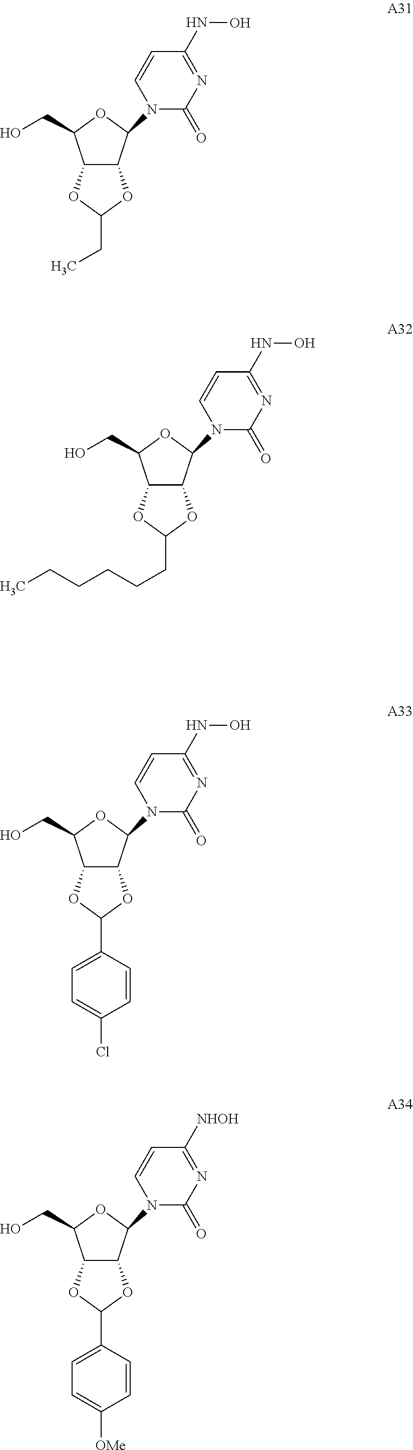

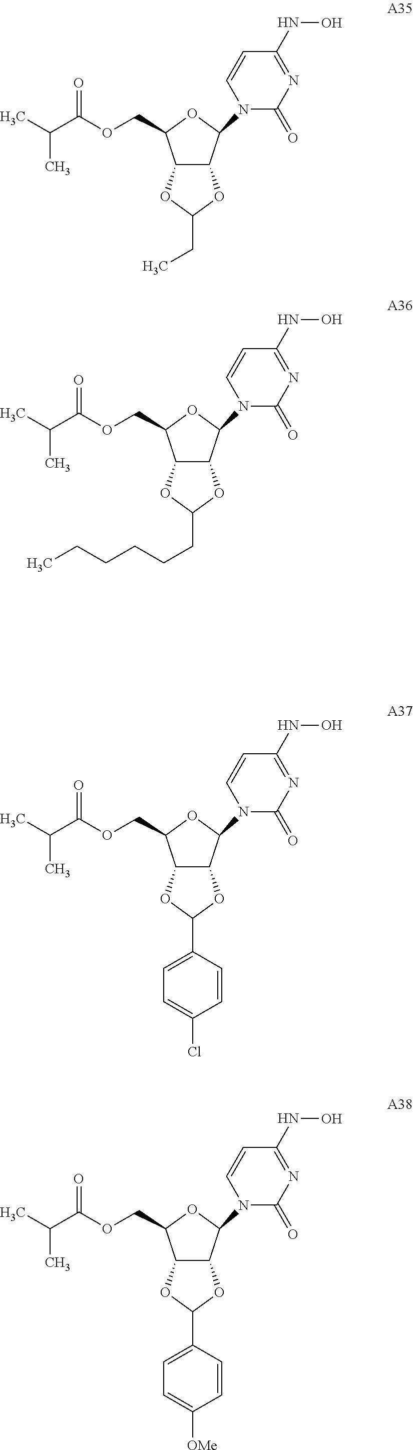

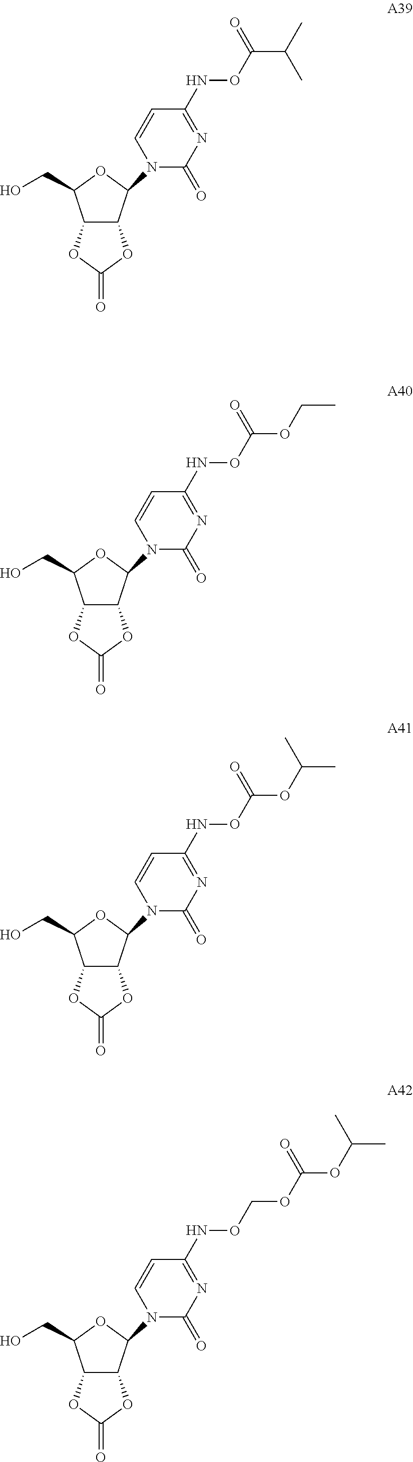 [SMILES: CC(C)C(=O)ONc1ccn([C@@H]2O[C@H](CO)[C@H]3OC(=O)O[C@H]32)c(=O)n1 ; CCOC(=O)ONc1ccn([C@@H]2O[C@H](CO)[C@H]3OC(=O)O[C@H]32)c(=O)n1 ; CC(C)OC(=O)ONc1ccn([C@@H]2O[C@H](CO)[C@H]3OC(=O)O[C@H]32)c(=O)n1 ; CC(C)OC(=O)OCONc1ccn([C@@H]2O[C@H](CO)[C@H]3OC(=O)O[C@H]32)c(=O)n1]

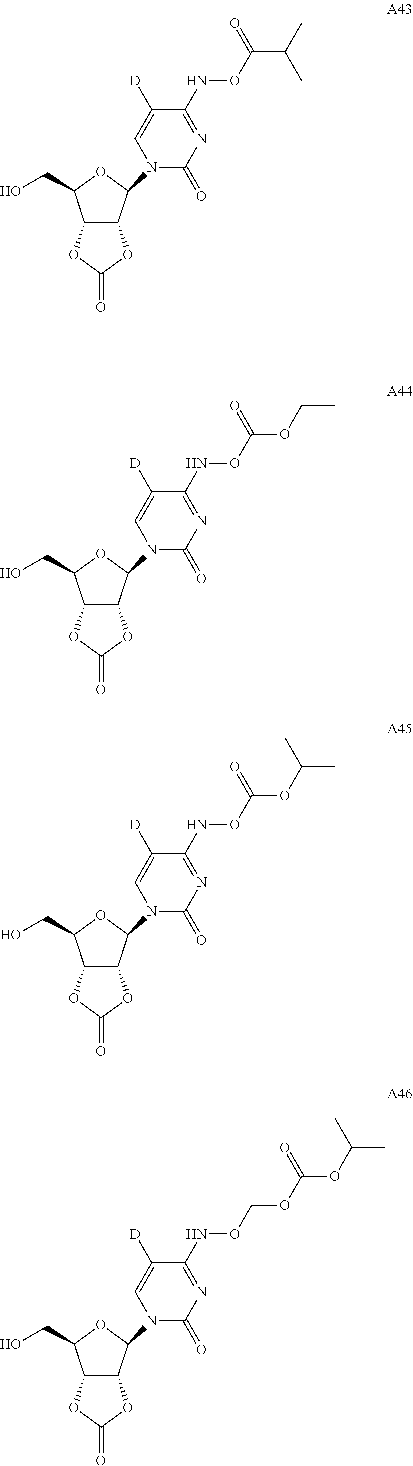 [SMILES: [2H]c1cn([C@@H]2O[C@H](CO)[C@H]3OC(=O)O[C@H]32)c(=O)nc1NOC(=O)C(C)C ; [2H]c1cn([C@@H]2O[C@H](CO)[C@H]3OC(=O)O[C@H]32)c(=O)nc1NOC(=O)OCC ; [2H]c1cn([C@@H]2O[C@H](CO)[C@H]3OC(=O)O[C@H]32)c(=O)nc1NOC(=O)OC(C)C ; [2H]c1cn([C@@H]2O[C@H](CO)[C@H]3OC(=O)O[C@H]32)c(=O)nc1NOCOC(=O)OC(C)C]

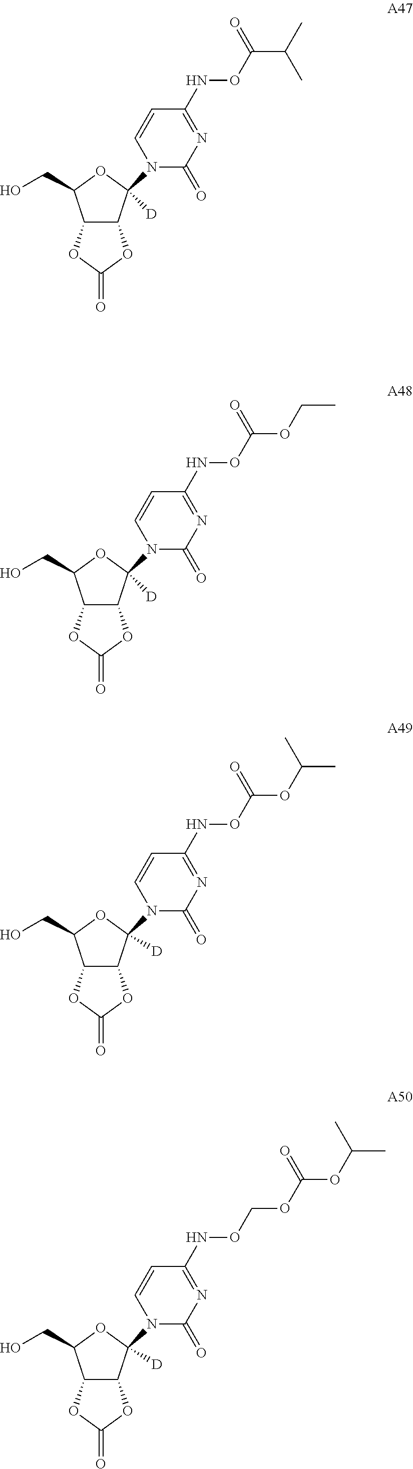 [SMILES: [2H][C@@]1(n2ccc(NOC(=O)C(C)C)nc2=O)O[C@H](CO)[C@H]2OC(=O)O[C@H]21 ; [2H][C@@]1(n2ccc(NOC(=O)OCC)nc2=O)O[C@H](CO)[C@H]2OC(=O)O[C@H]21 ; [2H][C@@]1(n2ccc(NOC(=O)OC(C)C)nc2=O)O[C@H](CO)[C@H]2OC(=O)O[C@H]21 ; [2H][C@@]1(n2ccc(NOCOC(=O)OC(C)C)nc2=O)O[C@H](CO)[C@H]2OC(=O)O[C@H]21]

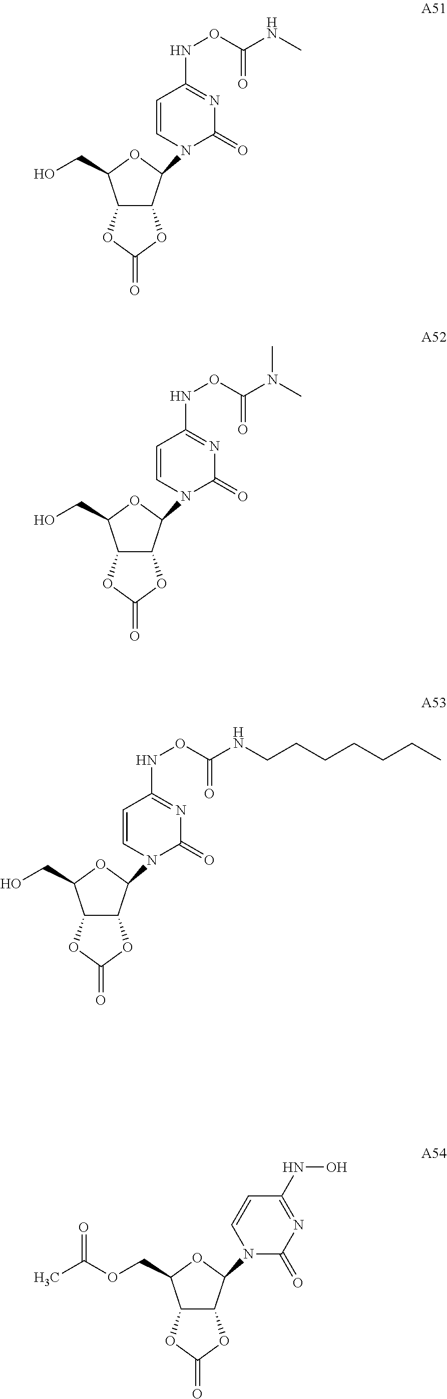

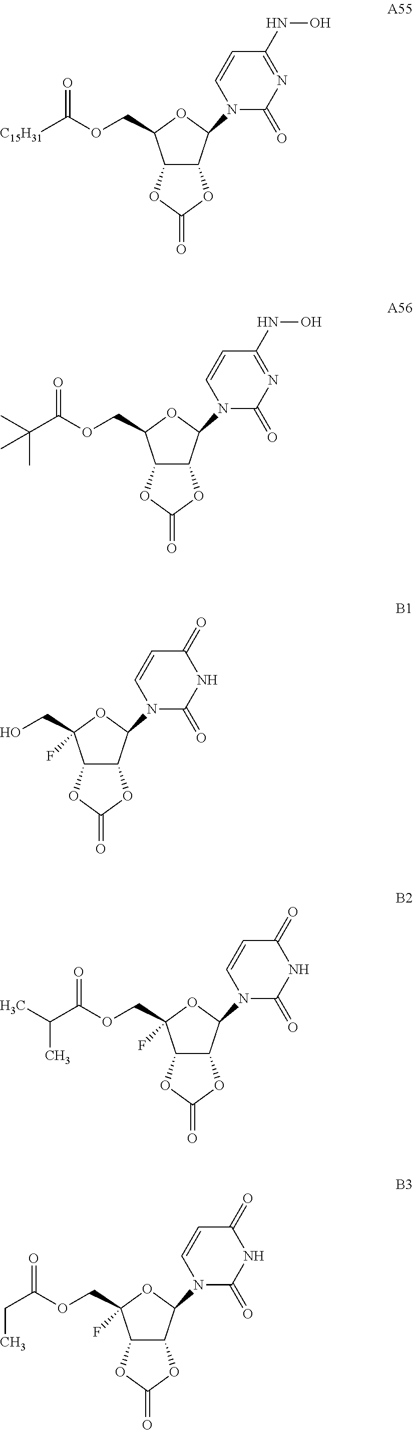

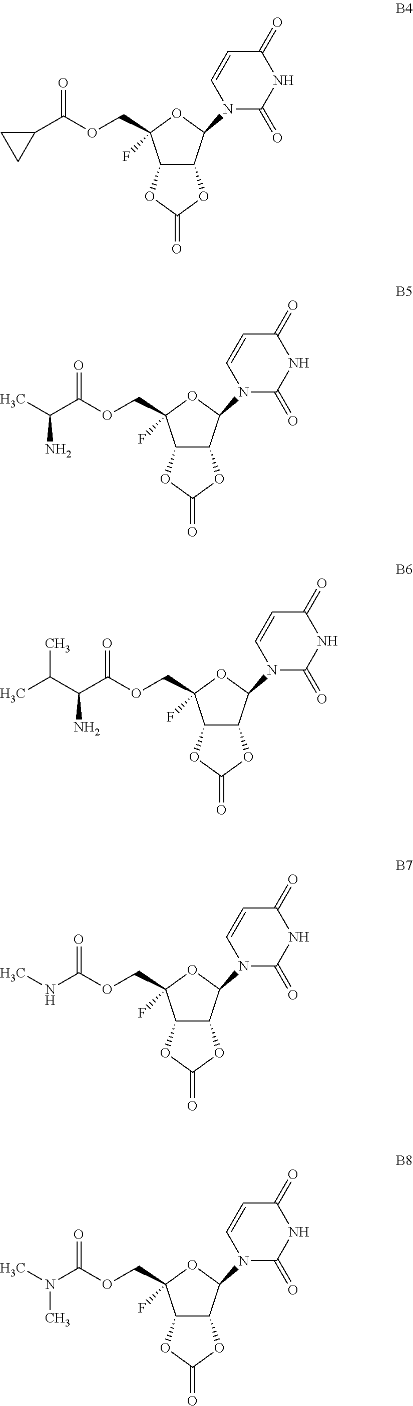 [SMILES: O=C1O[C@H]2[C@H](n3ccc(=O)[nH]c3=O)O[C@](F)(COC(=O)C3CC3)[C@H]2O1 ; C[C@H](N)C(=O)OC[C@@]1(F)O[C@@H](n2ccc(=O)[nH]c2=O)[C@@H]2OC(=O)O[C@@H]21 ; CC(C)[C@H](N)C(=O)OC[C@@]1(F)O[C@@H](n2ccc(=O)[nH]c2=O)[C@@H]2OC(=O)O[C@@H]21 ; CNC(=O)OC[C@@]1(F)O[C@@H](n2ccc(=O)[nH]c2=O)[C@@H]2OC(=O)O[C@@H]21 ; CN(C)C(=O)OC[C@@]1(F)O[C@@H](n2ccc(=O)[nH]c2=O)[C@@H]2OC(=O)O[C@@H]21]

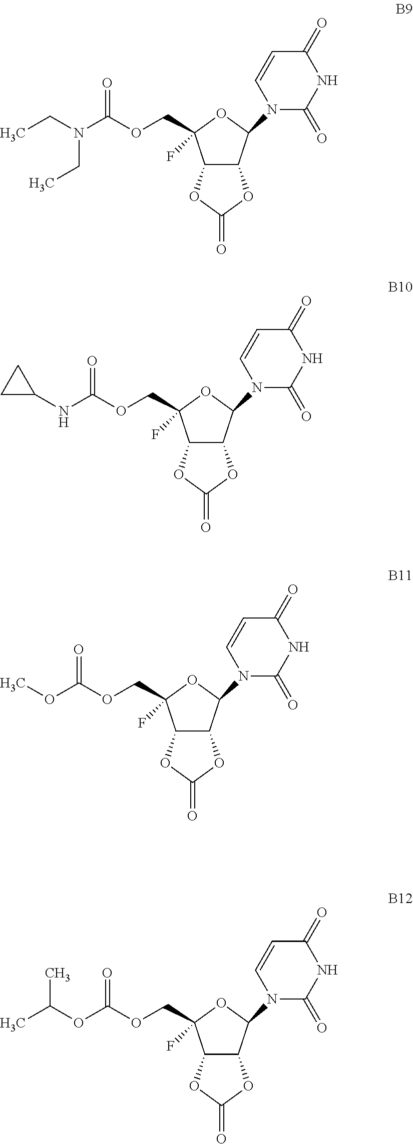 [SMILES: CCN(CC)C(=O)OC[C@@]1(F)O[C@@H](n2ccc(=O)[nH]c2=O)[C@@H]2OC(=O)O[C@@H]21 ; O=C(NC1CC1)OC[C@@]1(F)O[C@@H](n2ccc(=O)[nH]c2=O)[C@@H]2OC(=O)O[C@@H]21 ; COC(=O)OC[C@@]1(F)O[C@@H](n2ccc(=O)[nH]c2=O)[C@@H]2OC(=O)O[C@@H]21 ; CC(C)OC(=O)OC[C@@]1(F)O[C@@H](n2ccc(=O)[nH]c2=O)[C@@H]2OC(=O)O[C@@H]21]

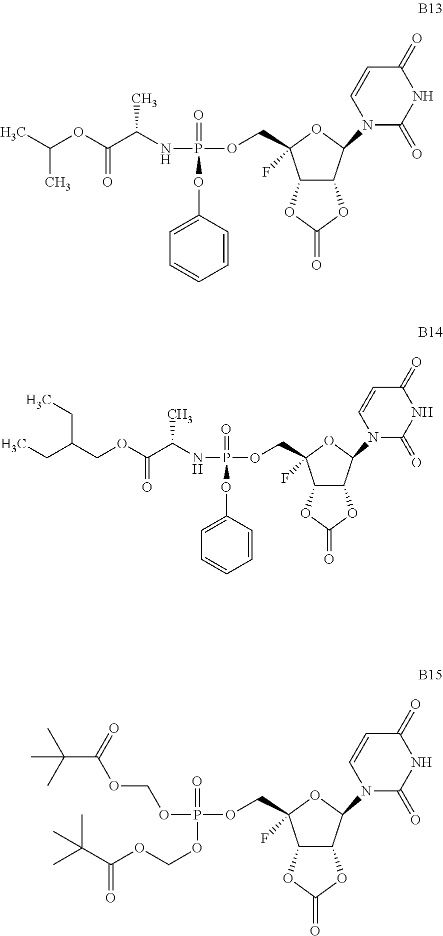 [SMILES: CC(C)OC(=O)[C@H](C)N[P@](=O)(OC[C@@]1(F)O[C@@H](n2ccc(=O)[nH]c2=O)[C@@H]2OC(=O)O[C@@H]21)Oc1ccccc1 ; CCC(CC)COC(=O)[C@H](C)N[P@](=O)(OC[C@@]1(F)O[C@@H](n2ccc(=O)[nH]c2=O)[C@@H]2OC(=O)O[C@@H]21)Oc1ccccc1 ; CC(C)(C)C(=O)OCOP(=O)(OCOC(=O)C(C)(C)C)OC[C@@]1(F)O[C@@H](n2ccc(=O)[nH]c2=O)[C@@H]2OC(=O)O[C@@H]21]

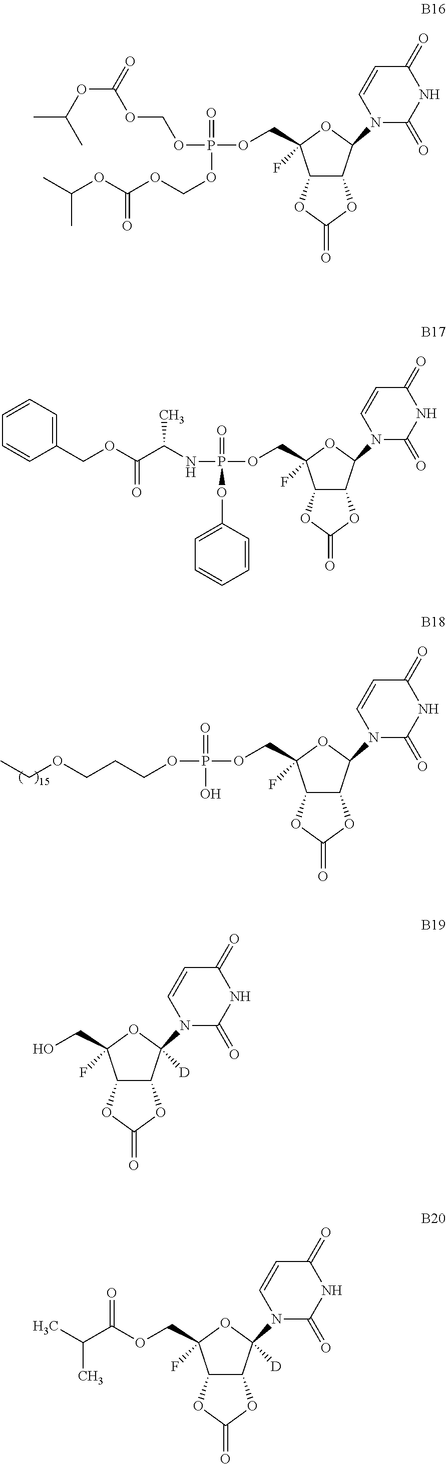

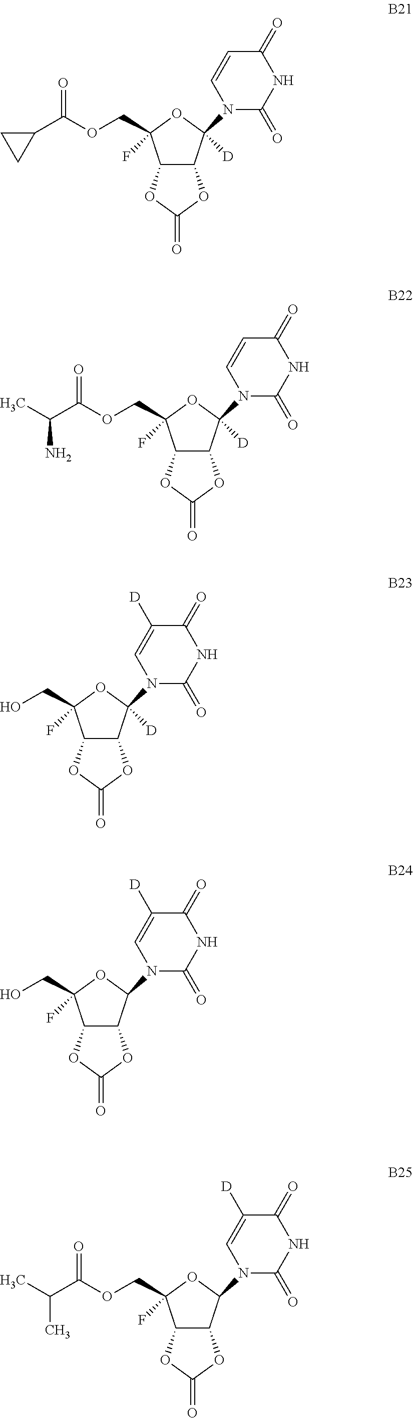 [SMILES: [2H][C@@]1(n2ccc(=O)[nH]c2=O)O[C@](F)(COC(=O)C2CC2)[C@H]2OC(=O)O[C@H]21 ; [2H][C@@]1(n2ccc(=O)[nH]c2=O)O[C@](F)(COC(=O)[C@H](C)N)[C@H]2OC(=O)O[C@H]21 ; [2H]c1cn([C@]2([2H])O[C@](F)(CO)[C@H]3OC(=O)O[C@H]32)c(=O)[nH]c1=O ; [2H]c1cn([C@@H]2O[C@](F)(CO)[C@H]3OC(=O)O[C@@H]23)c(=O)[nH]c1=O ; [2H]c1cn([C@@H]2O[C@](F)(COC(=O)C(C)C)[C@H]3OC(=O)O[C@@H]23)c(=O)[nH]c1=O]

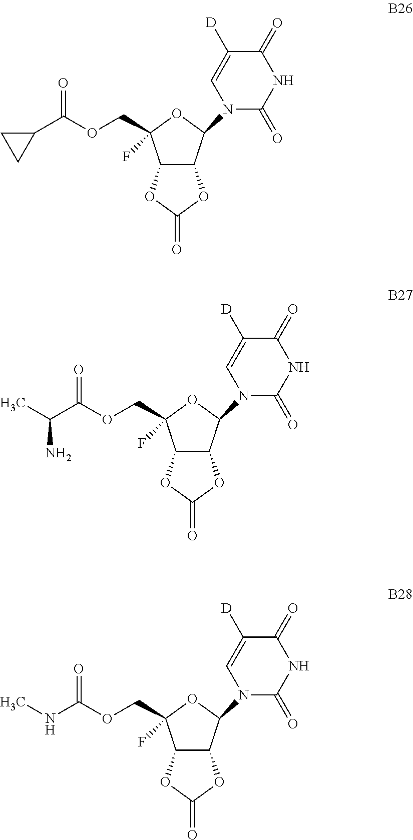 [SMILES: [2H]c1cn([C@@H]2O[C@](F)(COC(=O)C3CC3)[C@H]3OC(=O)O[C@@H]23)c(=O)[nH]c1=O ; [2H]c1cn([C@@H]2O[C@](F)(COC(=O)[C@H](C)N)[C@H]3OC(=O)O[C@@H]23)c(=O)[nH]c1=O ; [2H]c1cn([C@@H]2O[C@](F)(COC(=O)NC)[C@H]3OC(=O)O[C@@H]23)c(=O)[nH]c1=O]

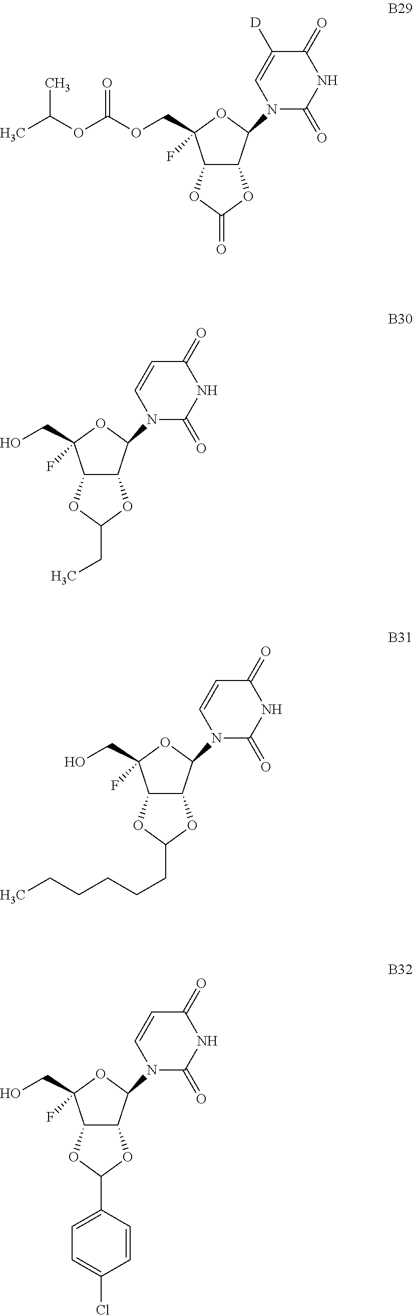

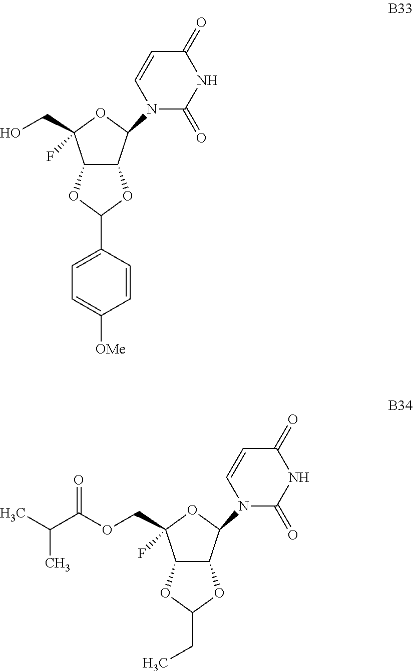

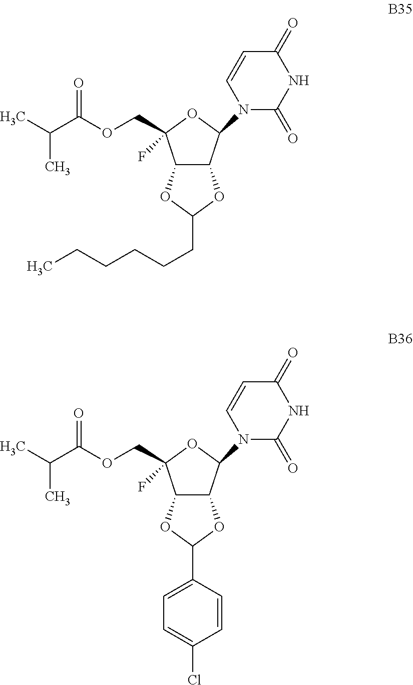

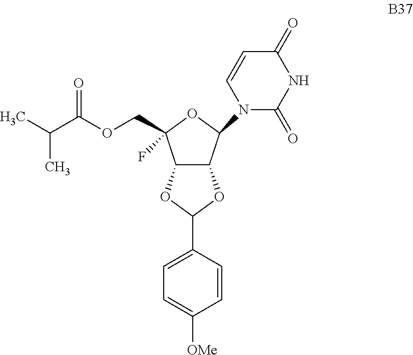

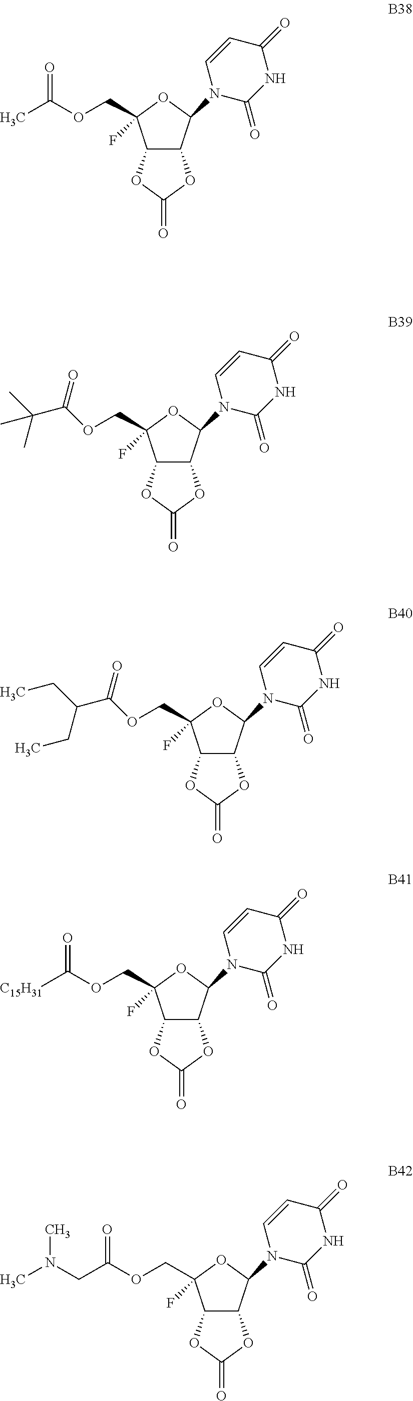

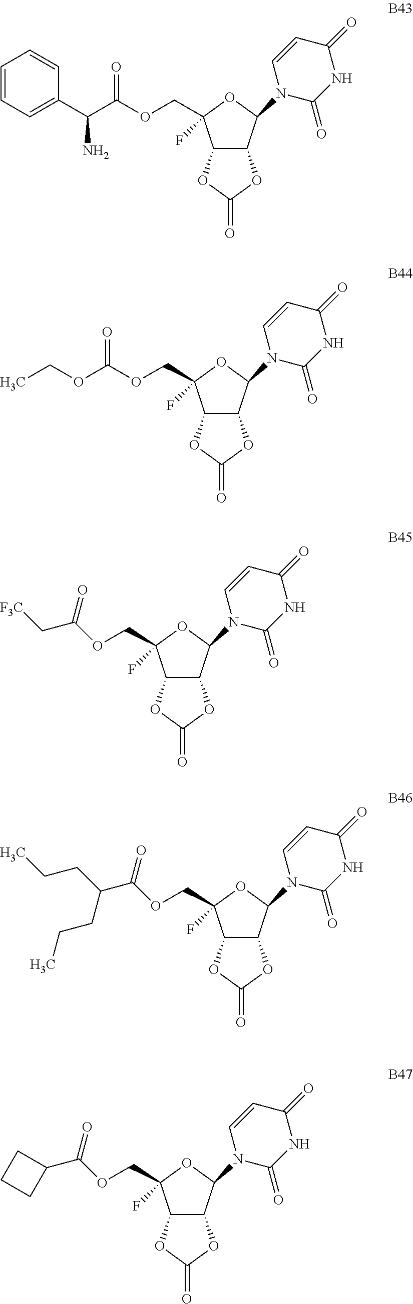 [SMILES: N[C@H](C(=O)OC[C@@]1(F)O[C@@H](n2ccc(=O)[nH]c2=O)[C@@H]2OC(=O)O[C@@H]21)c1ccccc1 ; CCOC(=O)OC[C@@]1(F)O[C@@H](n2ccc(=O)[nH]c2=O)[C@@H]2OC(=O)O[C@@H]21 ; O=C(CC(F)(F)F)OC[C@@]1(F)O[C@@H](n2ccc(=O)[nH]c2=O)[C@@H]2OC(=O)O[C@@H]21 ; CCCC(CCC)C(=O)OC[C@@]1(F)O[C@@H](n2ccc(=O)[nH]c2=O)[C@@H]2OC(=O)O[C@@H]21 ; O=C1O[C@H]2[C@H](n3ccc(=O)[nH]c3=O)O[C@](F)(COC(=O)C3CCC3)[C@H]2O1]

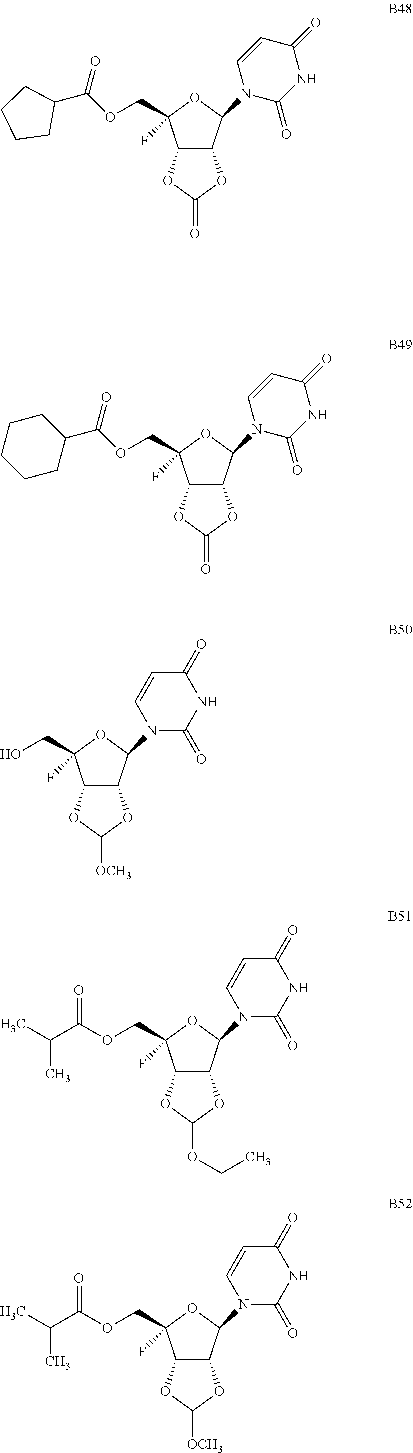

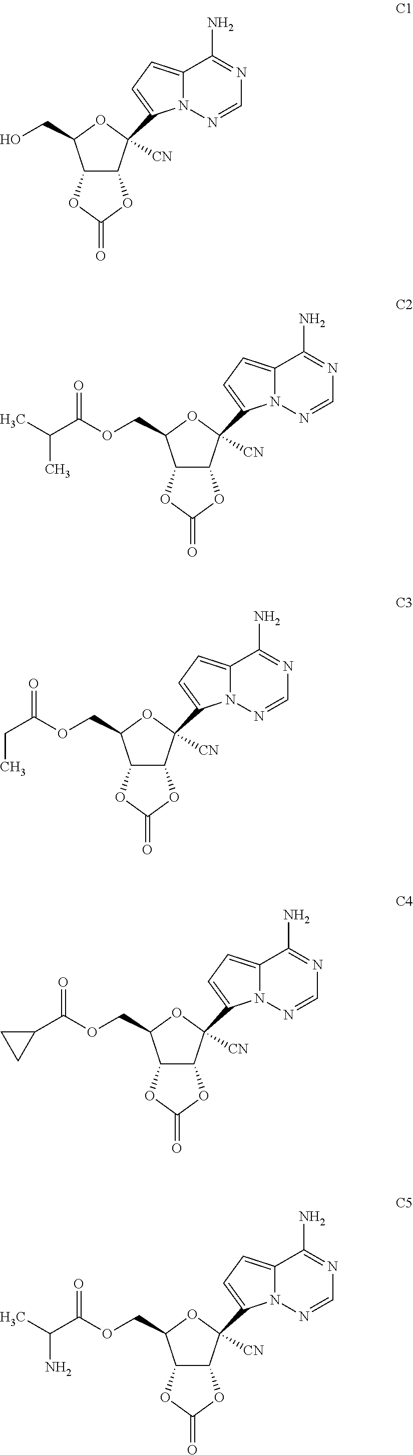 [SMILES: N#C[C@@]1(c2ccc3c(N)ncnn23)O[C@H](CO)[C@H]2OC(=O)O[C@H]21 ; CC(C)C(=O)OC[C@H]1O[C@@](C#N)(c2ccc3c(N)ncnn23)[C@@H]2OC(=O)O[C@@H]21 ; CCC(=O)OC[C@H]1O[C@@](C#N)(c2ccc3c(N)ncnn23)[C@@H]2OC(=O)O[C@@H]21 ; N#C[C@@]1(c2ccc3c(N)ncnn23)O[C@H](COC(=O)C2CC2)[C@H]2OC(=O)O[C@H]21 ; CC(N)C(=O)OC[C@H]1O[C@@](C#N)(c2ccc3c(N)ncnn23)[C@@H]2OC(=O)O[C@@H]21]

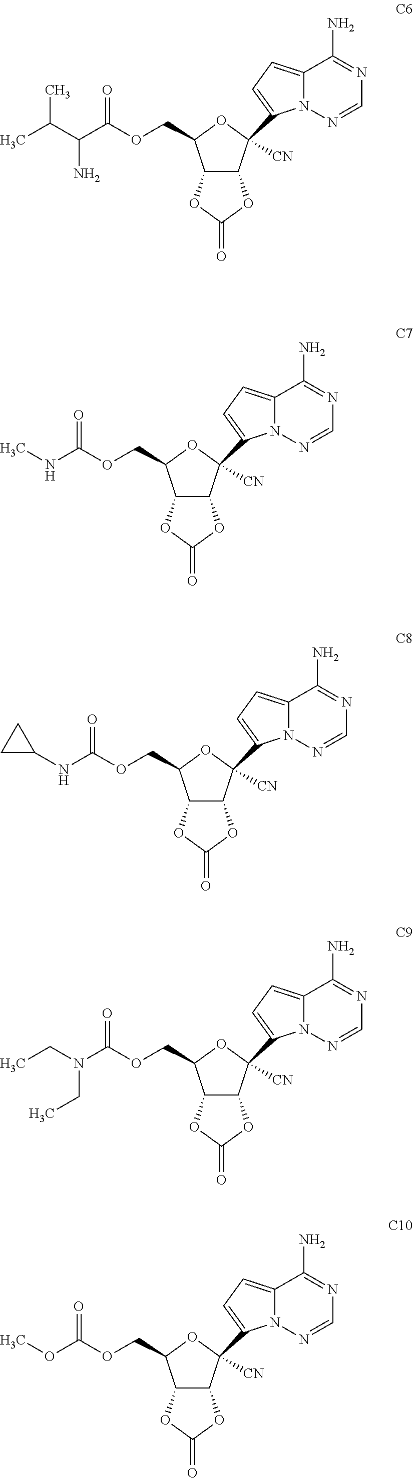 [SMILES: CC(C)C(N)C(=O)OC[C@H]1O[C@@](C#N)(c2ccc3c(N)ncnn23)[C@@H]2OC(=O)O[C@@H]21 ; CNC(=O)OC[C@H]1O[C@@](C#N)(c2ccc3c(N)ncnn23)[C@@H]2OC(=O)O[C@@H]21 ; N#C[C@@]1(c2ccc3c(N)ncnn23)O[C@H](COC(=O)NC2CC2)[C@H]2OC(=O)O[C@H]21 ; CCN(CC)C(=O)OC[C@H]1O[C@@](C#N)(c2ccc3c(N)ncnn23)[C@@H]2OC(=O)O[C@@H]21 ; COC(=O)OC[C@H]1O[C@@](C#N)(c2ccc3c(N)ncnn23)[C@@H]2OC(=O)O[C@@H]21]

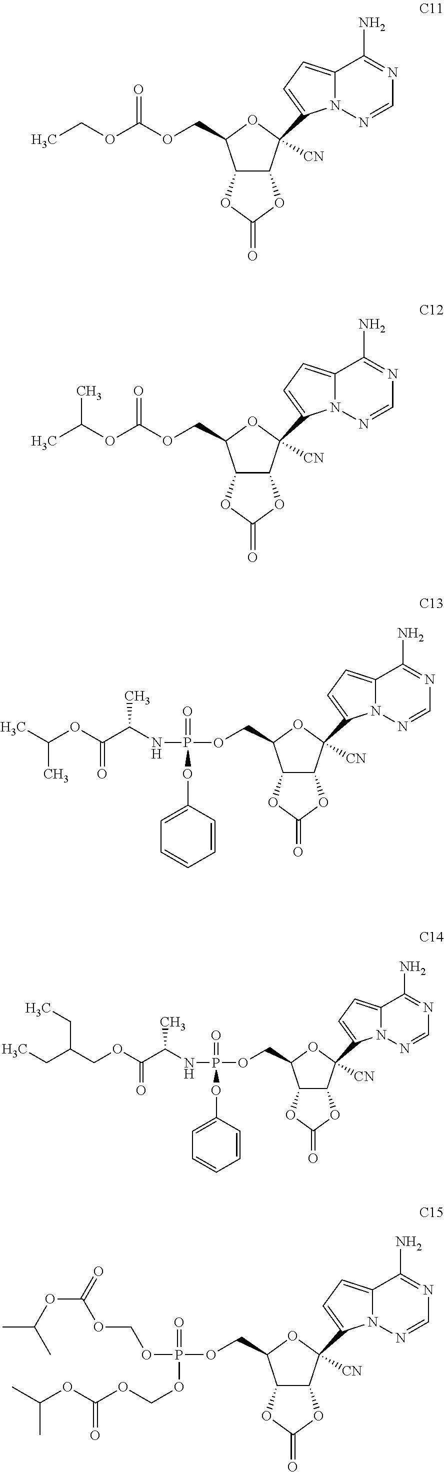 [SMILES: CCOC(=O)OC[C@H]1O[C@@](C#N)(c2ccc3c(N)ncnn23)[C@@H]2OC(=O)O[C@@H]21 ; CC(C)OC(=O)OC[C@H]1O[C@@](C#N)(c2ccc3c(N)ncnn23)[C@@H]2OC(=O)O[C@@H]21 ; CC(C)OC(=O)[C@H](C)N[P@](=O)(OC[C@H]1O[C@@](C#N)(c2ccc3c(N)ncnn23)[C@@H]2OC(=O)O[C@@H]21)Oc1ccccc1 ; CCC(CC)COC(=O)[C@H](C)N[P@](=O)(OC[C@H]1O[C@@](C#N)(c2ccc3c(N)ncnn23)[C@@H]2OC(=O)O[C@@H]21)Oc1ccccc1 ; CC(C)OC(=O)OCOP(=O)(OCOC(=O)OC(C)C)OC[C@H]1O[C@@](C#N)(c2ccc3c(N)ncnn23)[C@@H]2OC(=O)O[C@@H]21]

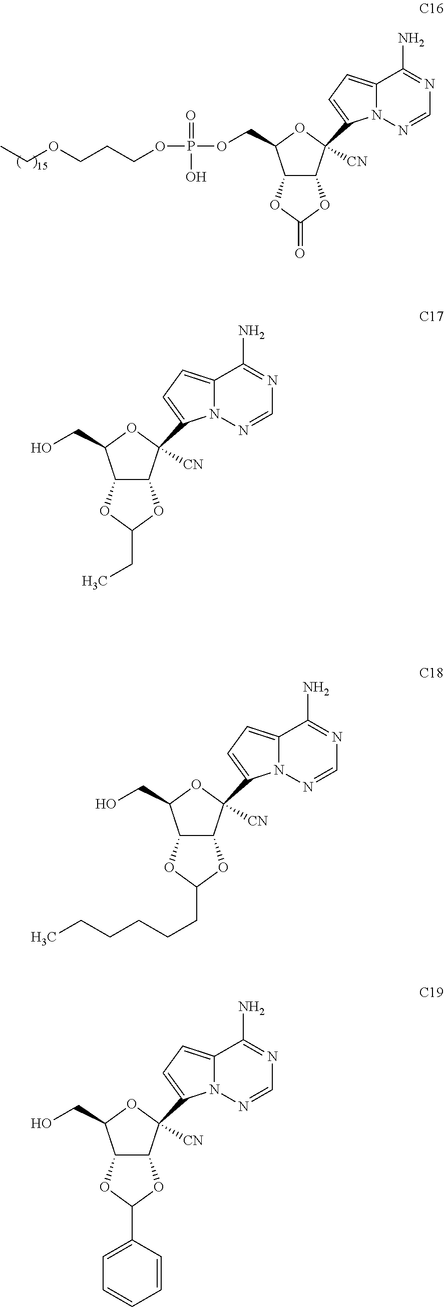

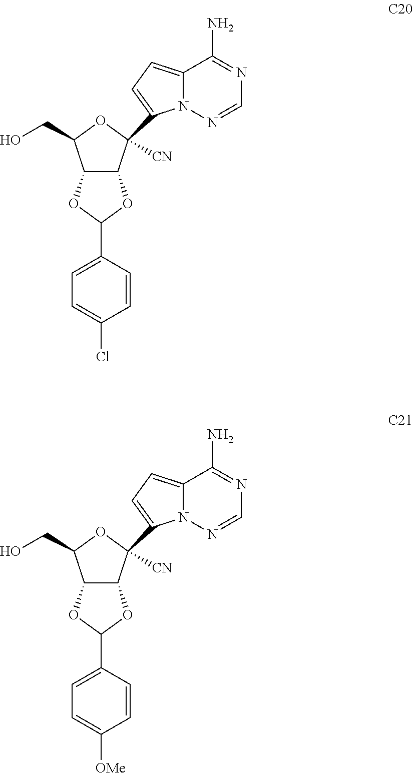

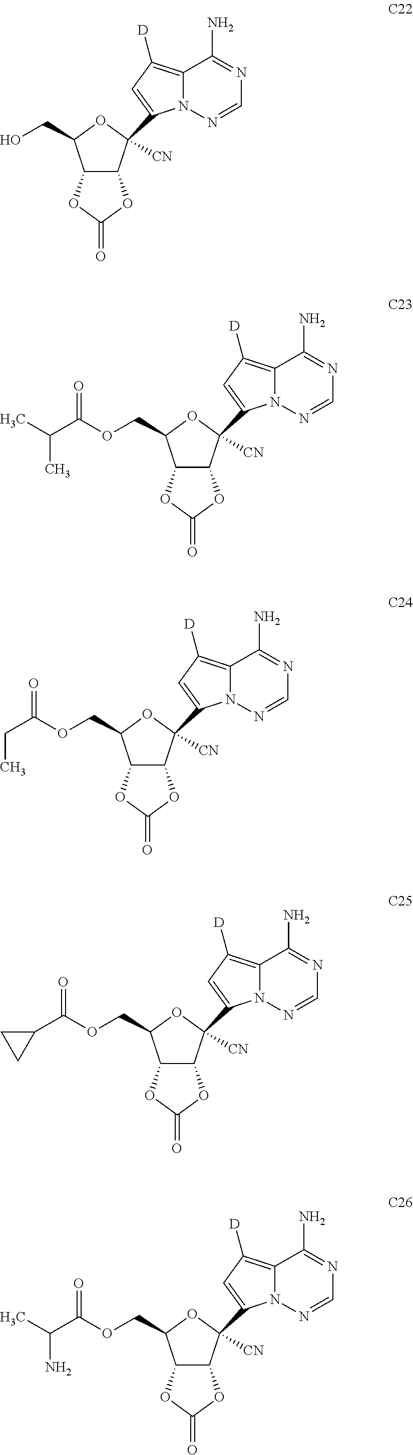 [SMILES: [2H]c1cc([C@]2(C#N)O[C@H](CO)[C@H]3OC(=O)O[C@H]32)n2ncnc(N)c12 ; [2H]c1cc([C@]2(C#N)O[C@H](COC(=O)C(C)C)[C@H]3OC(=O)O[C@H]32)n2ncnc(N)c12 ; [2H]c1cc([C@]2(C#N)O[C@H](COC(=O)CC)[C@H]3OC(=O)O[C@H]32)n2ncnc(N)c12 ; [2H]c1cc([C@]2(C#N)O[C@H](COC(=O)C3CC3)[C@H]3OC(=O)O[C@H]32)n2ncnc(N)c12 ; [2H]c1cc([C@]2(C#N)O[C@H](COC(=O)C(C)N)[C@H]3OC(=O)O[C@H]32)n2ncnc(N)c12]

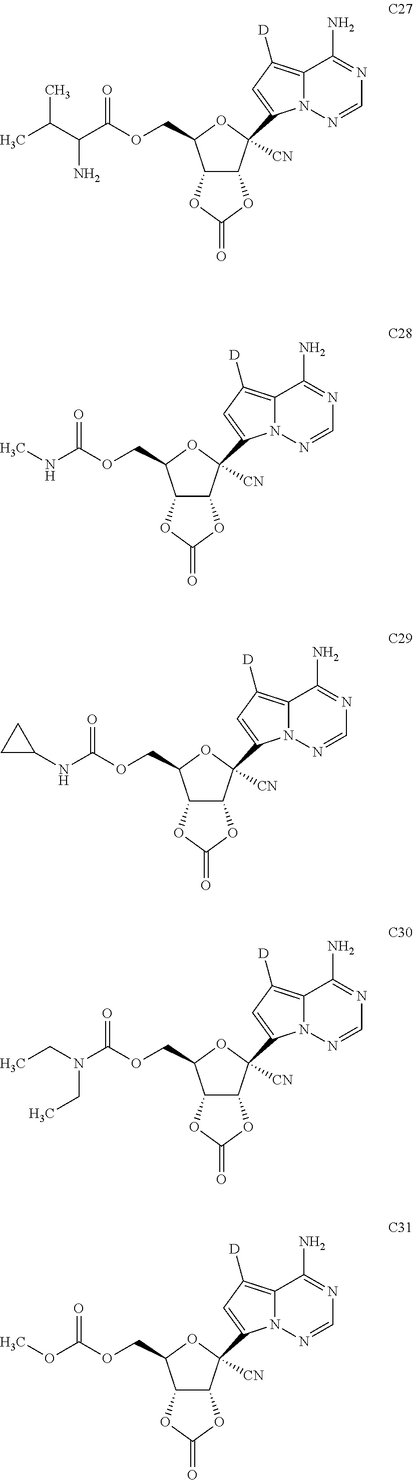 [SMILES: [2H]c1cc([C@]2(C#N)O[C@H](COC(=O)C(N)C(C)C)[C@H]3OC(=O)O[C@H]32)n2ncnc(N)c12 ; [2H]c1cc([C@]2(C#N)O[C@H](COC(=O)NC)[C@H]3OC(=O)O[C@H]32)n2ncnc(N)c12 ; [2H]c1cc([C@]2(C#N)O[C@H](COC(=O)NC3CC3)[C@H]3OC(=O)O[C@H]32)n2ncnc(N)c12 ; [2H]c1cc([C@]2(C#N)O[C@H](COC(=O)N(CC)CC)[C@H]3OC(=O)O[C@H]32)n2ncnc(N)c12 ; [2H]c1cc([C@]2(C#N)O[C@H](COC(=O)OC)[C@H]3OC(=O)O[C@H]32)n2ncnc(N)c12]

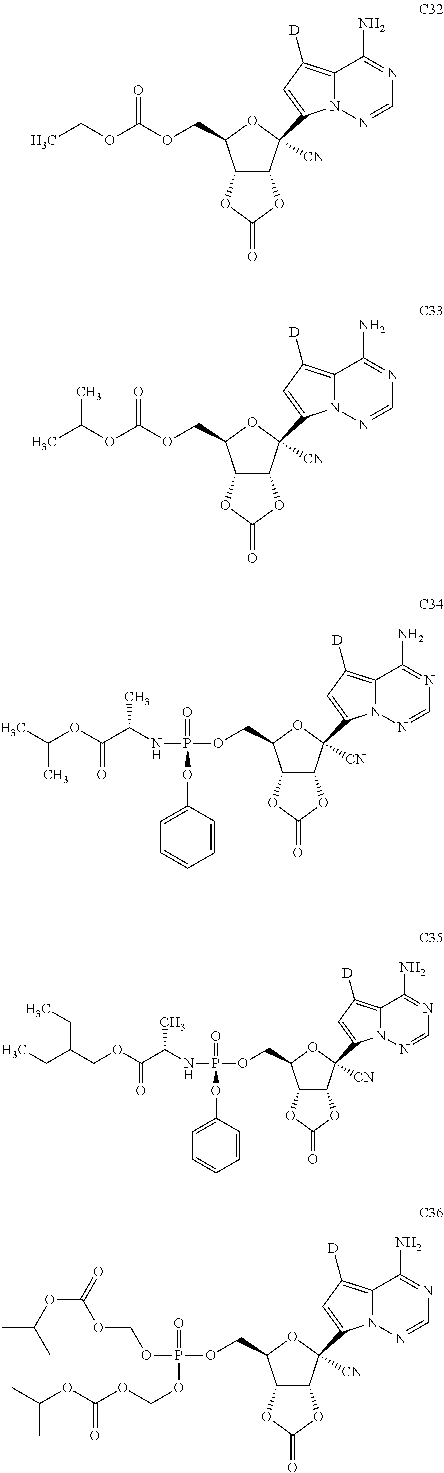 [SMILES: [2H]c1cc([C@]2(C#N)O[C@H](COC(=O)OCC)[C@H]3OC(=O)O[C@H]32)n2ncnc(N)c12 ; [2H]c1cc([C@]2(C#N)O[C@H](COC(=O)OC(C)C)[C@H]3OC(=O)O[C@H]32)n2ncnc(N)c12 ; [2H]c1cc([C@]2(C#N)O[C@H](CO[P@@](=O)(N[C@@H](C)C(=O)OC(C)C)Oc3ccccc3)[C@H]3OC(=O)O[C@H]32)n2ncnc(N)c12 ; [2H]c1cc([C@]2(C#N)O[C@H](CO[P@@](=O)(N[C@@H](C)C(=O)OCC(CC)CC)Oc3ccccc3)[C@H]3OC(=O)O[C@H]32)n2ncnc(N)c12 ; [2H]c1cc([C@]2(C#N)O[C@H](COP(=O)(OCOC(=O)OC(C)C)OCOC(=O)OC(C)C)[C@H]3OC(=O)O[C@H]32)n2ncnc(N)c12]

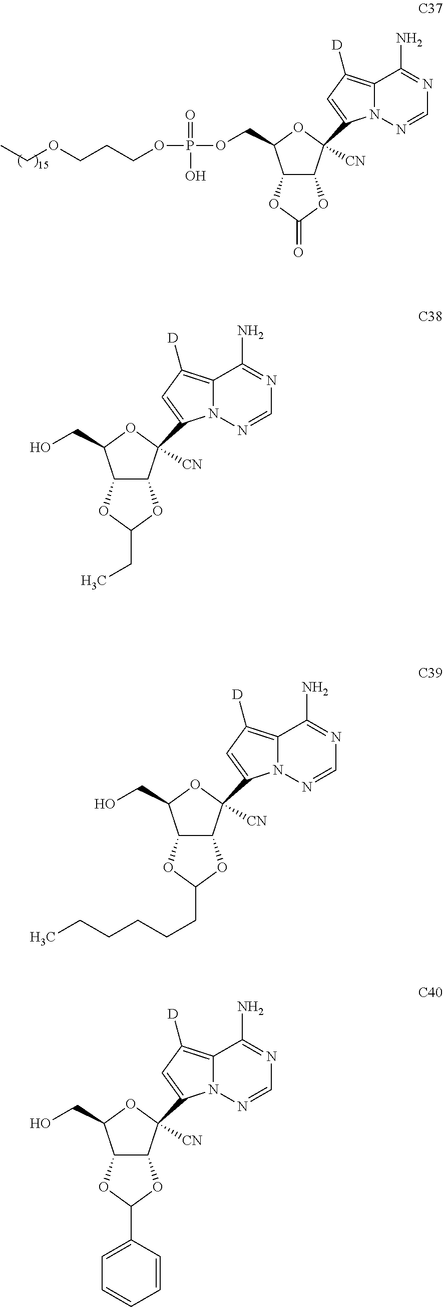

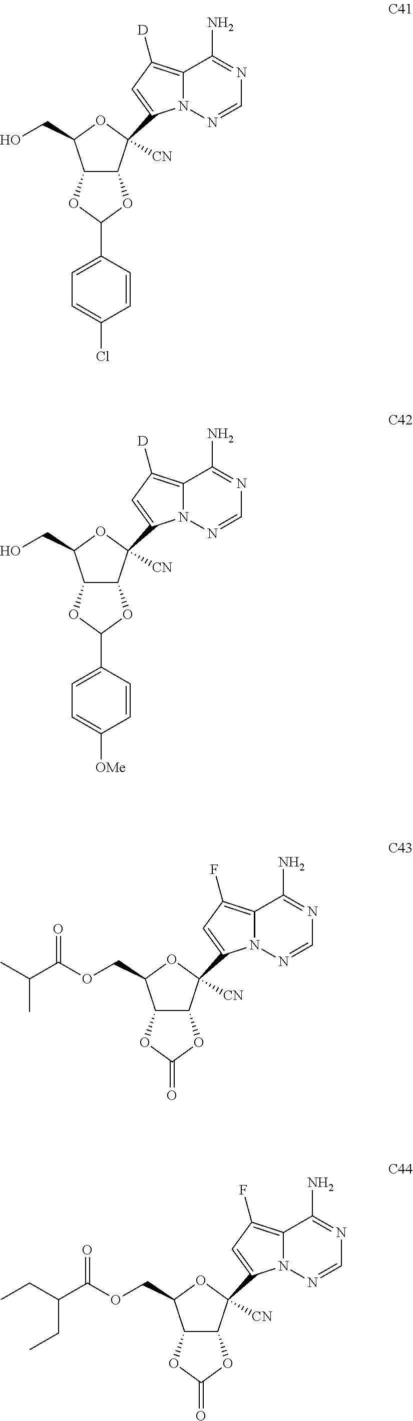

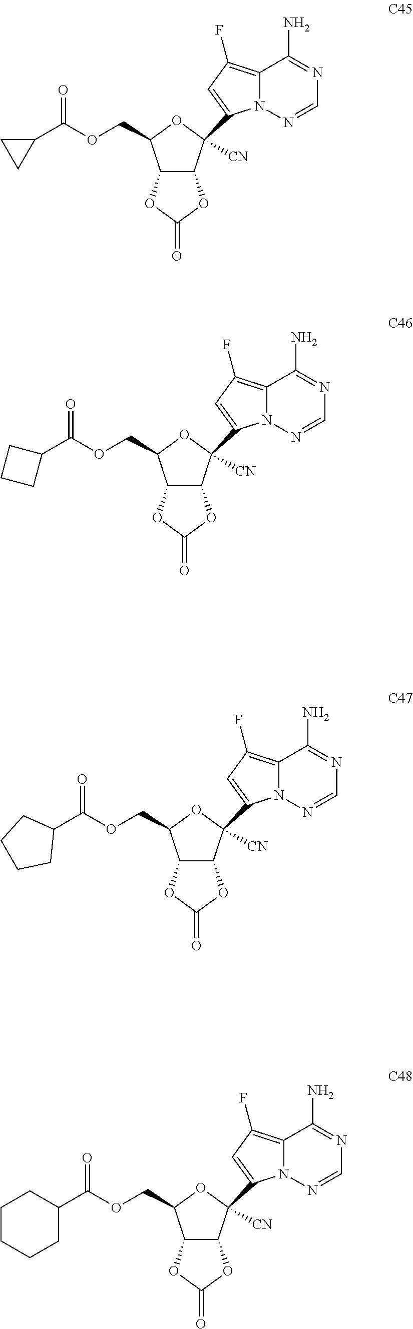 [SMILES: N#C[C@@]1(c2cc(F)c3c(N)ncnn23)O[C@H](COC(=O)C2CC2)[C@H]2OC(=O)O[C@H]21 ; N#C[C@@]1(c2cc(F)c3c(N)ncnn23)O[C@H](COC(=O)C2CCC2)[C@H]2OC(=O)O[C@H]21 ; N#C[C@@]1(c2cc(F)c3c(N)ncnn23)O[C@H](COC(=O)C2CCCC2)[C@H]2OC(=O)O[C@H]21 ; N#C[C@@]1(c2cc(F)c3c(N)ncnn23)O[C@H](COC(=O)C2CCCCC2)[C@H]2OC(=O)O[C@H]21]

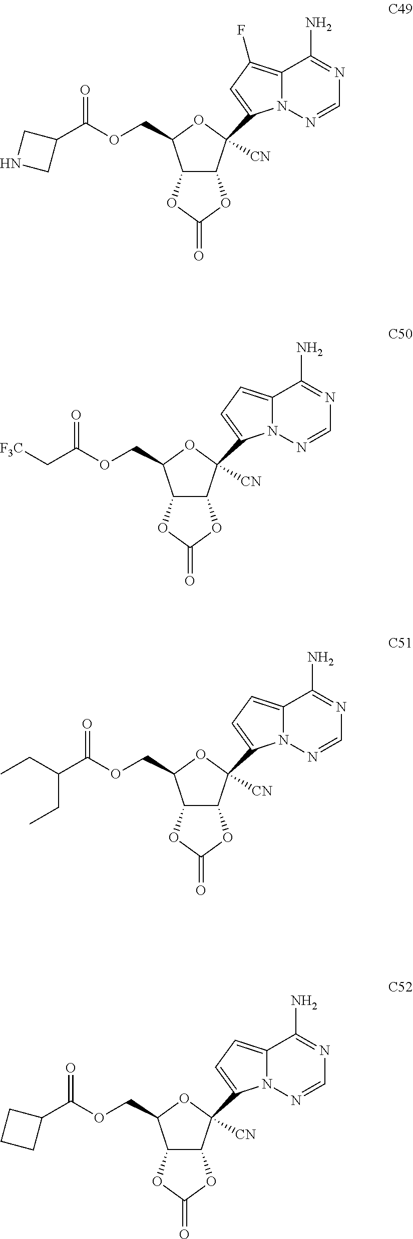 [SMILES: N#C[C@@]1(c2cc(F)c3c(N)ncnn23)O[C@H](COC(=O)C2CNC2)[C@H]2OC(=O)O[C@H]21 ; N#C[C@@]1(c2ccc3c(N)ncnn23)O[C@H](COC(=O)CC(F)(F)F)[C@H]2OC(=O)O[C@H]21 ; CCC(CC)C(=O)OC[C@H]1O[C@@](C#N)(c2ccc3c(N)ncnn23)[C@@H]2OC(=O)O[C@@H]21 ; N#C[C@@]1(c2ccc3c(N)ncnn23)O[C@H](COC(=O)C2CCC2)[C@H]2OC(=O)O[C@H]21]

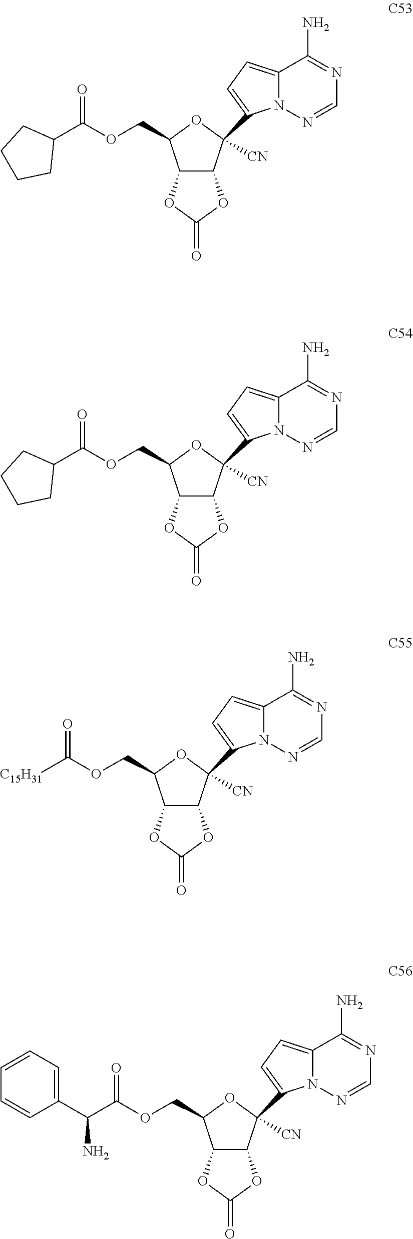

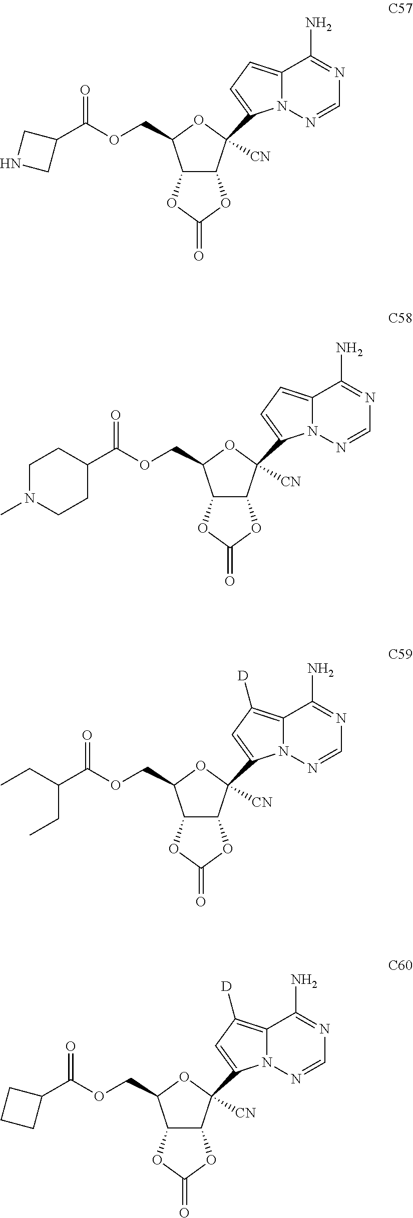 [SMILES: N#C[C@@]1(c2ccc3c(N)ncnn23)O[C@H](COC(=O)C2CNC2)[C@H]2OC(=O)O[C@H]21 ; CN1CCC(C(=O)OC[C@H]2O[C@@](C#N)(c3ccc4c(N)ncnn34)[C@@H]3OC(=O)O[C@@H]32)CC1 ; [2H]c1cc([C@]2(C#N)O[C@H](COC(=O)C(CC)CC)[C@H]3OC(=O)O[C@H]32)n2ncnc(N)c12 ; [2H]c1cc([C@]2(C#N)O[C@H](COC(=O)C3CCC3)[C@H]3OC(=O)O[C@H]32)n2ncnc(N)c12]

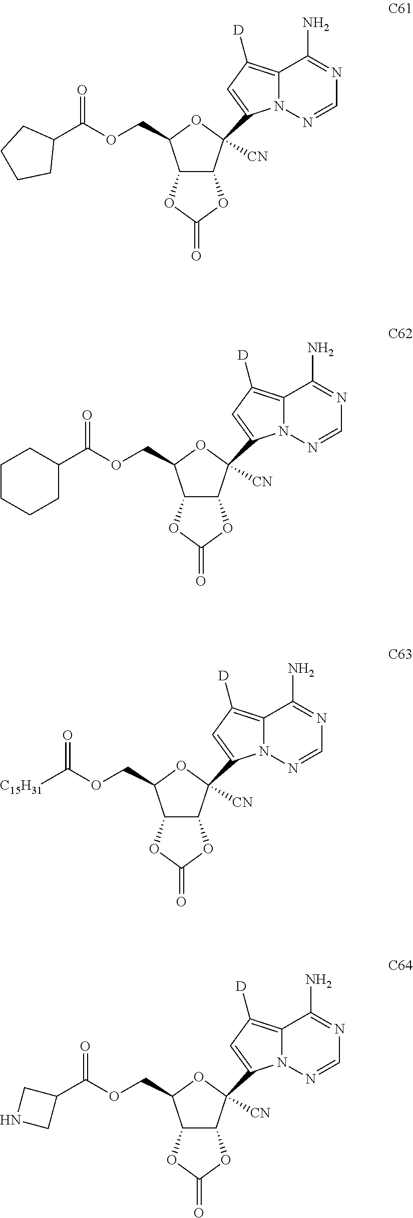

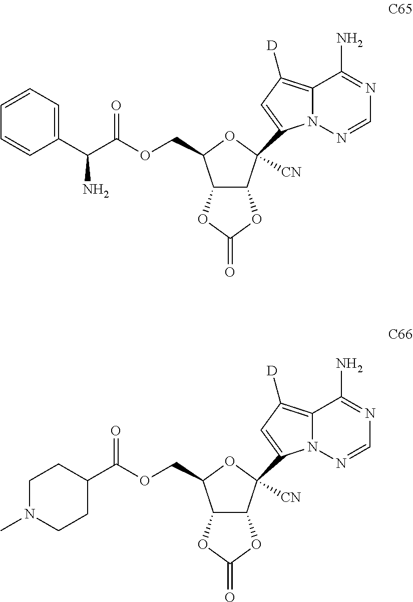 [SMILES: [2H]c1cc([C@]2(C#N)O[C@H](COC(=O)[C@@H](N)c3ccccc3)[C@H]3OC(=O)O[C@H]32)n2ncnc(N)c12 ; [2H]c1cc([C@]2(C#N)O[C@H](COC(=O)C3CCN(C)CC3)[C@H]3OC(=O)O[C@H]32)n2ncnc(N)c12]

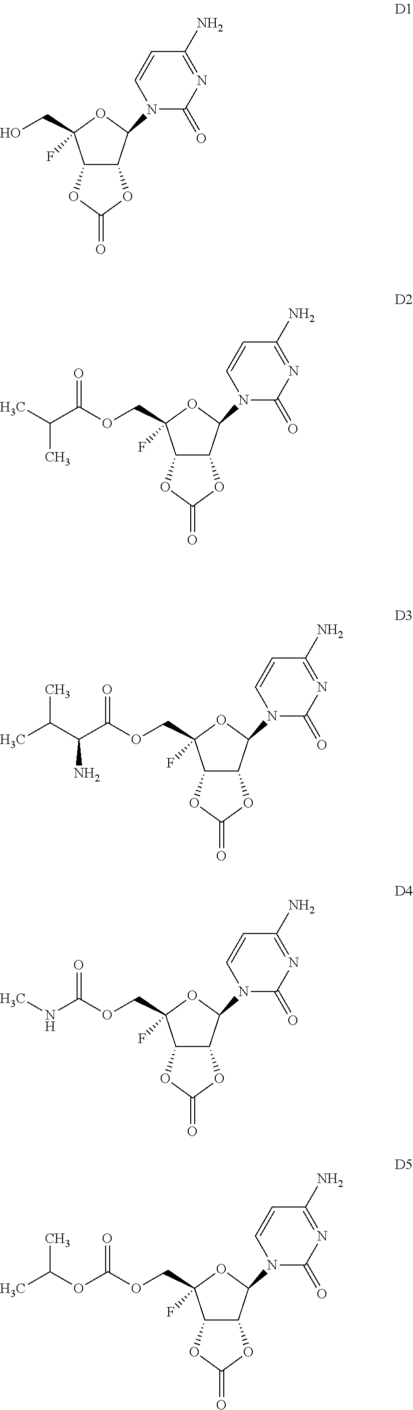 [SMILES: Nc1ccn([C@@H]2O[C@](F)(CO)[C@H]3OC(=O)O[C@@H]23)c(=O)n1 ; CC(C)C(=O)OC[C@@]1(F)O[C@@H](n2ccc(N)nc2=O)[C@@H]2OC(=O)O[C@@H]21 ; CC(C)[C@H](N)C(=O)OC[C@@]1(F)O[C@@H](n2ccc(N)nc2=O)[C@@H]2OC(=O)O[C@@H]21 ; CNC(=O)OC[C@@]1(F)O[C@@H](n2ccc(N)nc2=O)[C@@H]2OC(=O)O[C@@H]21 ; CC(C)OC(=O)OC[C@@]1(F)O[C@@H](n2ccc(N)nc2=O)[C@@H]2OC(=O)O[C@@H]21]

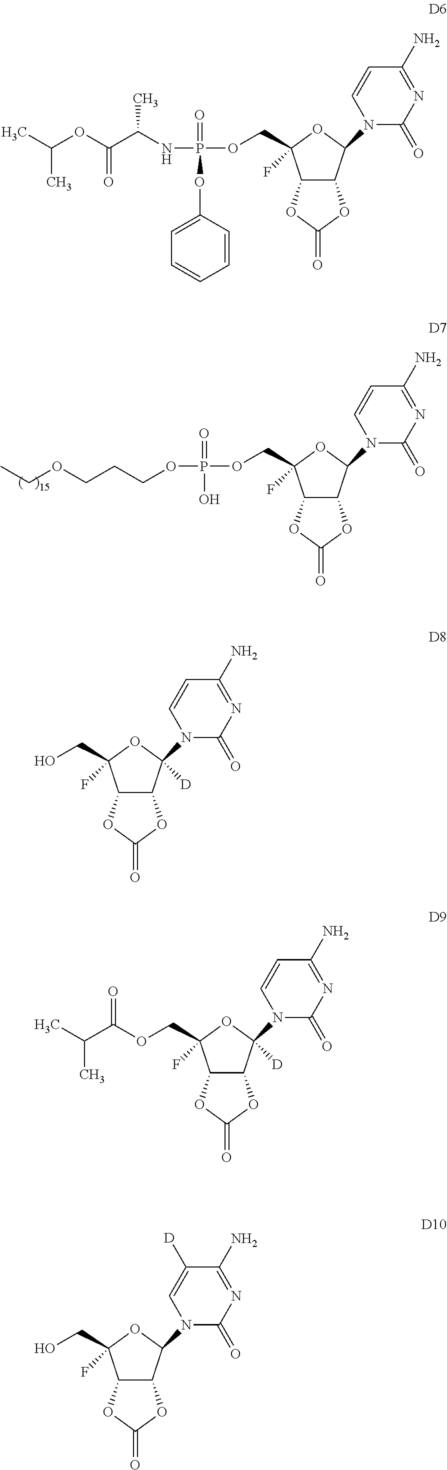

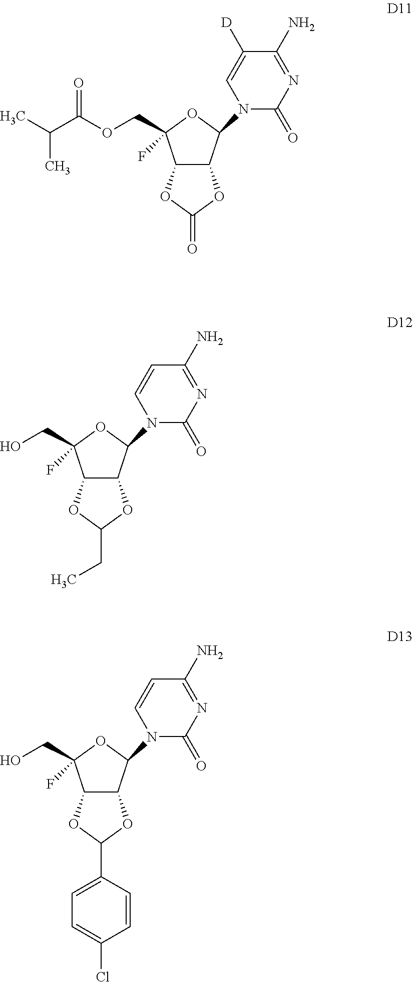

The compound represented by the formula I-III is equivalent to the compound represented by the formula and they are tautomers, wherein the hydroxylamine group at the 4-position of the pyrimidine base can be represented by an oxime group.

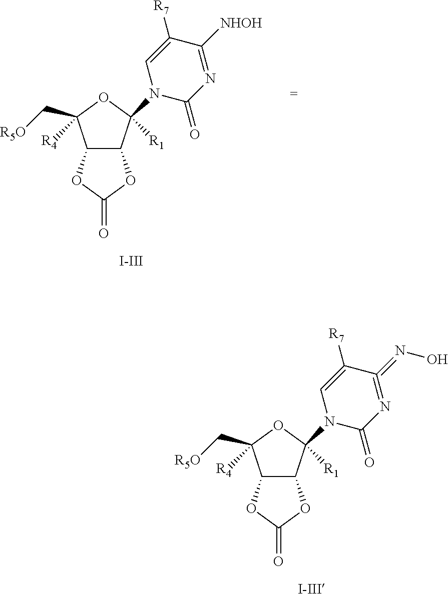

In some embodiments, the above-mentioned compounds or pharmaceutically acceptable salts thereof according to the present invention may exist in the form of crystalline hydrates, solvates or co-crystal compounds, and therefore, these crystalline hydrates, solvates and co-crystal compounds are also included within the scope of the present invention.

In some embodiments, the above compounds or pharmaceutically acceptable salts thereof according to the present invention may exist in the form of enantiomers, diastereomers or combinations thereof. These enantiomers, diastereomers and combinations thereof are also included within the scope of the present invention.

In a second aspect of the present invention, provided is a pharmaceutical composition comprising:
(a1) a first active ingredient, which is one or more selected from the group consisting of the above-mentioned compounds of formula I and pharmaceutically acceptable salts thereof: and
(b) a pharmaceutically acceptable carrier.

In a preferred embodiment, the composition may also contain (a2) a second active ingredient; which is one or more selected from the group consisting of antiviral active ingredients, corticosteroid anti-inflammatory drugs, adjuvant therapy drugs, etc.

In some embodiments, the antiviral active ingredient is one or more selected from the group consisting of interferon, RNA-dependent RNA polymerase inhibitors (such as Remdesivir (or GS-5734), Favipiravir, Galidesivir, GS-441524, NHC (EIDD-1931, EIDD-2801), 3CL Protease inhibitors (e.g., GC-376), lopinavir, ritonavir, nelfinavir, chloroquine, hydroxychloroquine, cyclosporine, carrimycin, baicalin, baicalein, forsythoside, chlorogenic acid, emodin, mycophenolic acid, mycophenolate mofetil, naphthoquine, ciclesonide, ribavirin, penciclovir, leflunomide, teriflunomide, nafamostat, nitazoxanide, darunavir, arbidol, camostat, niclosamide, baricitinib, ruxolitinib, dasatinib, saquinavir, beclabuvir, simeprevir, palivizumab, motavizumab, RSV-IGIV (RespiGam®), MEDI-557, A-60444 (RSV-604), MDT-637, BMS-433771, or a pharmaceutically acceptable salt thereof.

In some embodiments, the corticosteroid anti-inflammatory drug is one or more selected from the group consisting of dexamethasone, dexamethasone sodium phosphate, fluorometholone, fluorometholone acetate, loteprednol, loteprednol etabonate, hydrocortisone, prednisolone, fludrocortisone, triamcinolone, triamcinolone acetonide, betamethasone, beclomethasone dipropionate, methylprednisolone, fluocinolone, fluocinonide, flunisolide, fluocortin-21-butylate, flumethasone, flumethasone pivalate, budesonide, halobetasol propionate, mometasone furoate, fluticasone propionate, ciclesonide, or a pharmaceutically acceptable salt thereof.

In some embodiments, the adjuvant therapy drug is one or more selected from the group consisting of Zinc, fingolimod, vitamin C, olmesartan medoxomil, valsartan, losartan, thalidomide, glycyrrhizic acid, artemisinin, dihydroartemisinin, artesunate, artemisone, azithromycin, escin, and naproxen.

In a preferred embodiment, the pharmaceutical composition is prepared as a formulation.

In a preferred embodiment, the formulation includes oral formulations and non-oral formulations.

In a preferred embodiment, the formulation includes powders, granules, capsules, injections, inhalations, tinctures, oral liquids, tablets, lozenges, or drop pills.

In a third aspect of the present invention, provided is use of the above-mentioned compound of formula I or a pharmaceutically acceptable salt thereof, or the above-mentioned pharmaceutical composition, in preparation of a medicine, which is (a) an inhibitor for inhibiting virus replication; and/or (b) a medicine for treating, preventing and/or alleviating a disease caused by viral infection.

In a preferred embodiment, the virus is one or more selected from the group consisting of:
(1) coronaviruses, including coronaviruses that infect human: such as severe acute respiratory syndrome coronavirus (SARS-CoV), 2019 novel coronavirus (2019-nCoV or SARS-CoV-2), Middle East respiratory syndrome coronavirus (MERS-CoV), human coronavirus OC43, human coronavirus 229E, human coronavirus NL63, human coronavirus HKU1; and coronaviruses that infect animals: e.g. porcine epidemic diarrhea virus (PEDV), feline infectious peritonitis virus (FIFV);
(2) paramyxoviruses, such as paraflu virus, measles virus, respiratory syncytial virus (RSV);
(3) influenza viruses, such as influenza A virus, influenza B virus, influenza C virus, influenza D virus;
(4) flaviviridae viruses, such as hepatitis C virus (HCV), dengue virus (DENY), Zika virus (Zika);
(5) filoviruses, such as Marburg virus (MBV), Ebola virus (EBV), Cueva virus;
(6) bunyaviridae viruses, such as Bunyaviviruses, Phleboviruses, Nairoviruses, Hantaviruses;
(7) arenaviruses, such as Lassa fever virus (LASV), Junin virus (JUNV), Machupo virus (MACV), etc.

In a preferred embodiment, the virus is 2019 novel coronavirus (SARS-CoV-2).

In another preferred embodiment, the virus is an influenza virus.

In a preferred embodiment, the disease caused by viral infection is one or more selected from the group consisting of:
(D1) common cold, high-risk symptom infection, respiratory tract infection, pneumonia and complications thereof caused by human coronavirus infection;
(D2) porcine epidemic diarrhea caused by porcine epidemic diarrhea virus (PEDV);
(D3) feline infectious peritonitis caused by feline coronavirus (FIFV);
(D4) common cold, high-risk symptom infection, respiratory tract infection, pneumonia and complications thereof caused by human respiratory syncytial virus (RSV) infection;
(D5) common cold, high-risk symptom infection, respiratory tract infection, pneumonia and complications thereof caused by influenza virus infection;
(D6) chronic hepatitis C and complications thereof caused by hepatitis C virus (HCV);
(D7) dengue fever and complications thereof caused by dengue virus (DENY);
(D8) infection and complications thereof caused by Zika virus (Zika);
(D9) hemorrhagic fever and complications thereof caused by Marburg virus (MBV), and Ebola virus (EBV);
(D10) infection and complications thereof caused by Bunyaviridae viruses;
(D11) infection and complications thereof caused by arenaviruses.

In a preferred embodiment, the disease caused by viral infection is a disease caused by 2019 novel coronavirus (SARS-CoV-2) infection. In particular, the disease caused by the 2019 novel coronavirus infection is one or more selected from the group consisting of respiratory tract infection, pneumonia and complications thereof.

In another preferred embodiment, the disease caused by viral infection is a disease caused by influenza virus infection. In particular, the disease caused by influenza virus infection is one or more selected from the group consisting of common cold, high-risk symptom infection, respiratory tract infection, pneumonia and complications thereof.

It should be understood that within the scope of the present invention, the above-mentioned technical features of the present invention and the technical features specifically described hereinafter (such as in examples) can be combined with each other to form new or preferred technical solutions. Due to space limitations, no more tautology here.

### DETAILED EMBODIMENTS

After extensive and in-depth research and extensive screening, the present inventors unexpectedly developed a class of nucleoside prodrugs with good pharmacokinetic properties. Experimental results revealed that the nucleoside prodrug of the present invention has high oral bioavailability with significant antiviral activity, and is expected to have obvious advantages in treatment of infections by various viruses such as 2019 novel coronavirus (SARS-CoV-2), influenza virus, respiratory syncytial virus, etc., and the present invention has been completed on this basis.

Specifically, the present invention discloses the nucleoside analog prodrug represented by formula (I), a composition thereof and use thereof in antiviral, in preparation of a virus inhibitor, and its use in the preparation of medicines for treating diseases, conditions or indications caused by viral infections. The viruses are coronaviruses (SARS, MERS, SARS-CoV-2, porcine epidemic diarrhea virus, feline infectious peritonitis virus, etc.), paramyxovirus (paraflu virus, measles virus, respiratory syncytial virus, etc.), influenza virus, flaviviridae virus (hepatitis C virus, dengue virus, Zika virus, etc.), filoviruses (Ebola virus, Marburg virus), bunya virus and/or arenavirus.

Specifically, the present invention discloses the use of nucleoside analog prodrugs represented by formula (I) and their compositions in antiviral, for example, in the preparation of (a) an inhibitor of replication of anticoronavirus (SARS, MERS, SARS-CoV-2, porcine epidemic diarrhea virus, feline infectious peritonitis virus, etc.), paramyxovirus (paraflu virus, measles virus, respiratory syncytial virus, etc.), influenza virus, flaviviridae virus (hepatitis C virus, dengue virus, Zika virus, etc.), filoviruses (Ebola virus, Marburg virus), bunya virus and/or arenavirus; and/or (b) medicines for treating and/or preventing and alleviating diseases caused by human coronaviruses (SARS, MERS, SARS-CoV-2, porcine epidemic diarrhea virus, feline infectious peritonitis virus, etc.), paramyxovirus (paraflu virus, measles virus, respiratory syncytial virus, etc.), influenza virus, flaviviridae virus (hepatitis C virus, dengue virus, Zika virus, etc.), filoviruses (Ebola virus, Marburg virus), bunya virus and/or arenavirus infection. The nucleoside analog prodrug represented by formula (I) has a high exposure to nucleoside metabolites in the body, and has a strong inhibitory effect on the replication of SARS-CoV-2, influenza virus, respiratory syncytial virus, etc., thus it has a good clinical application prospect.

### Terminology

As used herein, “compounds of the present invention”, “nucleoside analogs of the present invention”, “nucleoside analog prodrugs of the present invention”, “nucleoside prodrugs of the present invention”, “active compounds of the present invention”, “antiviral nucleoside analogs of the present invention” can be used interchangeably, referring to nucleoside analogs with excellent antiviral effects in vivo or in vitro, including the compound of formula I, or a pharmaceutically acceptable salt thereof, or a solvate thereof, or a prodrug thereof, or a combination thereof.

As used herein, “a formulation of the present invention” refers to a formulation containing a nucleoside analog of the present invention.

As used herein, the term “comprising” or its variants such as “comprises” or “includes” and the like, are understood to include the stated elements or constituents but not to exclude other elements or other constituents.

As used herein, the terms “novel coronavirus”, “2019-nCoV” or “SARS-CoV-2” are used interchangeably, and the 2019 novel coronavirus is the seventh coronavirus known to infect humans and causes new coronary pneumonia (COVID-19), is one of the serious infectious diseases threatening human health worldwide.

As used herein, “halogen” generally refers to fluorine, chlorine, bromine and iodine; preferably fluorine, chlorine or bromine; more preferably fluorine or chlorine.

As used herein, the term “Cₙ-Cₘ” and “Cₙ₋ₘ” are used interchangeably, refer to having n to m carbon atoms.

As used herein, the term “C₁₋₁₈ alkyl” alone or as part of a composite group refers to a straight or branched saturated hydrocarbon group containing 1-18 carbon atoms, for example, C₁₋₆ alkyl means, for example methyl, ethyl, n-propyl, isopropyl, n-butyl, isobutyl, tert-butyl, sec-butyl, n-pentyl, 1-ethylpropyl, isopentyl, neopentyl, isohexyl, 3-methylpentyl or n-hexyl, etc., preferably methyl, ethyl, n-propyl, isopropyl, butyl, isobutyl or tert-butyl.

As used herein, the term “halogenated C₁₋₆ alkyl” alone or as part of a composite group means that the hydrogen atoms of the above C₁₋₆ alkyl are substituted by one or more identical or different halogen atoms, for example, trifluoromethyl, fluoromethyl, difluoromethyl, chloromethyl, bromomethyl, dichlorofluoromethyl, chloroethyl, bromopropyl, 2-chlorobutyl, pentafluoroethyl, etc.

As used herein, the term “C₁₋₆ alkoxy” alone or as part of a composite group refers to a straight or branched chain alkoxy containing 1-6 carbon atoms, for example, methoxy, ethoxy, n-propoxy, isopropoxy, n-butoxy, isobutoxy, tert-butoxy, sec-butoxy, n-pentoxy, isopentyloxy, neopentyloxy, isohexyloxy, 3-methylpentoxy, n-hexyloxy, etc., preferably methoxy, ethoxy, n-propoxy, isopropoxy, n-butoxy, isobutoxy, or tert-butoxy.

As used herein, the term “C₃₋₂₀ cycloalkyl” alone or as part of a composite group refers to a cyclic saturated hydrocarbon group containing 1-20 carbon atoms, for example, C₃₋₈ cycloalkyl, such as cyclopropyl, cyclobutyl, cyclopentyl, cyclohexyl, cycloheptyl, cyclooctyl, etc.

As used herein, the term “C₁₋₂₀ alkanoyl” alone or as part of a composite group refers to RC(═O)—, wherein R is selected from the group consisting of hydrogen and C₁₋₁₉ alkyl, the examples of C₁₋₂₀ alkanoyl are formyl, acetyl, propionyl, butyryl, isobutyryl, valeryl, tert-butyryl, hexanoyl etc. Sometimes alkanoyl can also be called alkylcarbonyl, such as C₁₋₄ alkylcarbonyl.

As used herein, the term “C₂₋₆alkenyl” alone or as part of a composite group refers to a straight or branched unsaturated hydrocarbon group containing 1-3 double bonds and 2-6 carbon atoms, including both cis-configuration and trans-configuration, for example, vinyl, 1-propenyl, 2-propenyl, 1-methyl-1-propenyl, 2-methyl-1-propenyl, 2-methyl-2-propenyl, 1-butenyl, 2-butenyl, 3 -butenyl, 1-pentenyl, 2-pentenyl, 3-pentenyl, 4-pentenyl, 1,3-butadienyl, 1,3-pentadienyl, 1-hexenyl, 2-hexenyl, 3-hexenyl, 4-hexenyl, 5-hexenyl, 3,3-dimethyl-1-propenyl or 2-ethyl- 1-propenyl, etc.

As used herein, the term “C₂₋₆ alkynyl” alone or as part of a composite group refers to a straight or branched alkynyl group containing 2 to 6 carbon atoms, for example, ethynyl, 2-propynyl, 2-butynyl, 3-butynyl, 1-methyl-2-propynyl, 2-pentynyl or 2-hexynyl, etc. As used herein, the term “amino C₁₋₂₀ alkanoyl” alone or as part of a composite group refers to a C₁₋₂₀ alkanoyl group in which one hydrogen is substituted by an amino group (—NH₂), such as —CONH₂, —COCH₂NH₂, —COCH₂CH₂NH₂, etc.

As used herein, the term “C₁₋₂₀ alkylamino” alone or as part of a composite group refers to a group obtained by replacing one hydrogen on an amino group (—NH₂) with a C₁₋₂₀ alkyl group, such as —NHCH₃, —NHCH₂CH₃ etc. Alkylamino is sometimes referred to as alkylamine group.

As used herein, the term “C₁₋₆ cycloalkylamino” alone or as part of a complex group refers to a group obtained by replacing one hydrogen on an amino group (—NH₂) with a C₁₋₆ cycloalkyl group, such as cyclopropylamino, cyclobutylamino, cyclopentylamino, cyclohexylamino, etc.

As used herein, the term “C₁₋₂₀ alkylamino C₁₋₆ alkanoyl” alone or as part of a composite group refers to a C₁₋₆ alkanoyl group in which one hydrogen is replaced by a C₁₋₂₀ alkylamino group, such as —CONHCH₃, —COCH₂NHCH₃, —COCH₂CH₂NHCH₂CH₃, etc.

As used herein, the term “C₁₋₆ cycloalkylamino C₁₋₆ alkanoyl” alone or as part of a composite group refers to a C₁₋₆ alkanoyl in which one hydrogen is replaced by a C₁₋₆ cycloalkylamino, such as cyclopropylamino C₁₋₆ alkanoyl, cyclobutylamino C₁₋₆ alkanoyl, cyclopentylamino C₁₋₆ alkanoyl, cyclohexylamino C₁₋₆ alkanoyl, etc.

As used herein, the term “C₁₋₂₀ dialkylamino” alone or as part of a composite group refers to a group in which two hydrogens on the amino group (—NH₂) are independently replaced by C₁₋₂₀ alkyl group, such as dimethylamino, diethylamino, methylethylamino, etc. Sometimes a C₁₋₆ dialkylamino group can also be called a di(C₁₋₆ alkyl)amine group, such as a di(C₁₋₄ alkyl)amine group.

As used herein, the term “C₁₋₂₀ dialkylamino C₁₋₆ alkanoyl” alone or as part of a composite group refers to a C₁₋₆ alkanoyl wherein one hydrogen is replaced by a C₁₋₂₀ dialkylamino, such as —CON(CH₃)₂, —CON(CH₂CH₃)₂, —COCH₂N(CH₂CH₃)₂, —COCH₂CH₂N(CH₂CH₃)₂, etc.

As used herein, the term “C₁₋₆ alkoxy C₁₋₆ alkyl” alone or as part of a composite group refers to a C₁₋₆ alkyl wherein one hydrogen is replaced by a C₁₋₆ alkoxy, such as —CH₂OCH₃, —CH₂CH₂OCH₂CH₃, etc.

As used herein, the term “amino C₁₋₆ alkyl” alone or as part of a composite group refers to a C₁₋₆ alkyl in which one hydrogen is replaced by an amino group (—NH₂), such as —CH₂NH₂, —CH₂CH₂NH₂, —CH(NH₂)CH₃, —CH₂CH₂CH₂NH₂ or —CH₂CH₂CH₂CH₂NH₂ etc.

As used herein, the term “hydroxyl C₁₋₆ alkyl” alone or as part of a composite group refers to a C₁₋₆ alkyl on which one hydrogen is replaced by a hydroxy group, such as —CH₂OH, —CH₂CH₂OH, —CH(OH)CH₃, —CH₂CH₂CH₂OH, —CH₂CH₂CH₂CH₂OH, or —CH₂CH(CH₃)CH₂OH, etc.

As used herein, the term “C₁₋₄ alkylcarbonyloxy” alone or as part of a composite group refers to a C₁₋₄ alkyl C(═O)O— group, for example, CH₃C(═O)O—, CH₃CH₂C(═O)O—, CH₃CH₂CH₂C(═O)O—, etc.

As used herein, the term “C₁₋₄ alkoxycarbonyl” alone or as part of a composite group refers to a C₁₋₄ alkyl-OC(═O)— group, for example, CH₃OC(═O)—, CH₃CH₂OC(═O)—, CH₃CH₂CH₂OC(═O)—, etc.

As used herein, the term “C₁₋₂₀ alkoxy C₁₋₆ alkanoyl” alone or as part of a composite group refers to a C₁₋₆ alkanoyl in which one hydrogen is replaced by a C₁₋₂₀ alkoxy, for example, CH₃OC(═O)—, CH₃CH₂OC(═O)—, (CH₃)₂CHOC(═O)—, CH₃OCH₂C(═O)—, etc.

As used herein, the term “C₁₋₆ alkoxycarbonyloxymethylene” alone or as part of a composite group refers to a C₁₋₆ alkyl-OC(═O)OCH₂- group, for example, CH₃OC(═O)OCH₂-, CH₃CH₂OC(═O)OCH₂-, (CH₃)₂CHOC(═O)OCH₂-, etc.

As used herein, the term “amino acid” refers to naturally occurring and synthetic amino acids, such as L-valine, L-alanine, L-phenylalanine, L-phenylglycine, D-valine, D-alanine, the amino acid is connected to the core structure through an ester bond formed by the carbonyl on its carboxyl and the oxygen connected with R₅.

As used herein, the term “C₆₋₂₀ aryl” alone or as part of a composite group refers to a monocyclic or polycyclic (e.g., bicyclic or tricyclic) aromatic ring having 6-20 carbon atoms in the ring and containing no heteroatoms, for example, C₆₋₁₂ aryl, C₆₋₁₀ aryl, such as phenyl, naphthyl, phenanthrenyl, anthracenyl, etc.

As used herein, the term “5-15 membered heteroaryl” alone or as part of a composite group refers to a monocyclic or polycyclic (e.g., bicyclic or tricyclic) aromatic rings having 5-15 atoms in the ring and containing 1-4 heteroatoms selected from the group consisting of nitrogen, oxygen and sulfur in the ring, for example, 5-10 membered heteroaryl, 5-6 membered heteroaryl, such as pyrrolyl, furyl, thienyl, imidazolyl, pyrazolyl, oxazolyl, isoxazolyl, thiazolyl, isothiazolyl, triazolyl, oxadiazolyl, thiadiazolyl, pyridyl, pyridazinyl, pyrimidinyl, pyrazinyl, indolyl, isoindolyl, indazolyl, benzotriazolyl, benzothienyl, isobenzothienyl, benzofuryl, benzoisofuryl, benzimidazolyl, benzoxazolyl, benzoisoxazolyl, benzothiazolyl, benzisothiazolyl, benzothiadiazolyl, indolazinyl, purinyl, imidazopyridinyl, imidazopyrimidinyl, imidazopyrazinyl, imidazopyridazinyl, imidazotriazinyl, pyrazolopyridyl, pyrazolopyrimidinyl, pyrazolopyrazinyl, pyrazolopyrazinyl, pyrazolotriazinyl, pyrrolopyridinyl, pyrrolopyrimidinyl, pyrrolopyridazinyl, pyrrolopyrazinyl and pyrrolotriazinyl, etc.

Herein, all features or conditions defined in the form of numerical ranges are for the sake of brevity and convenience only. Accordingly, the description of numerical ranges should be considered to encompass and specifically disclose all possible subranges and individual numerical values, especially integer values, within such ranges. For example, a range of “1-6” should be deemed to have specifically disclosed all subranges such as 1 to 6, 2 to 6, 2 to 5, 3 to 5, 4 to 6, 3 to 6, etc., specifically subranges defined by all integer values, and should be deemed to have specifically disclosed individual values such as 1, 2, 3, 4, 5, 6 etc. within the specifically disclosed ranges.

Unless otherwise indicated, the foregoing method of interpretation is applicable to all content throughout the present invention, whether broad or not.

Where a quantity or other value or parameter is expressed in the form of a range, a preferred range, or a series of upper and lower limits, it is to be understood that all ranges consisting of any upper or preferred value of that range and lower limit or preferred value of that range have been specifically disclosed, whether or not those ranges are separately disclosed. Further, whenever a numerical range is referred to herein, unless otherwise indicated, such range shall include its endpoints and all integers and fractions within the range.

### Coronavirus

Coronavirus (CoV) belongs to order Nidovirales, family Coronaviridae. It is an enveloped positive-strand RNA virus, and its subfamily includes four genera, α, β, δ and γ coronavirus.

Among the coronaviruses currently known to infect humans, HCoV-229E and HCoV-NL63 belong to a coronaviruses, and HCoV-OC43, SARS-CoV, HCoV-HKU1, MERS-CoV and SARS-CoV-2 are all β coronaviruses.

The genome of this type of viruses is a single-stranded positive-strand RNA, which is one of the largest among RNA viruses. The genome encodes proteins include replicase, spike protein, membrane protein, envelope protein and nucleocapsid protein. In the initial stage of viral replication, the genome is translated into two peptide chains of several thousand amino acids, i.e., the precursor polyprotein, and then the precursor polyprotein is cleaved by proteases to generate non-structural proteins (such as RNA polymerase and unwinding enzymes) and structural proteins (such as spike proteins) and accessory proteins.

### Influenza Virus

Influenza viruses are referred to as flu viruses for short, and common influenza viruses are divided into A, B, C and D types. Influenza viruses can cause infection and disease in many animals such as human, poultry, pig, horse, and bat, and are the pathogens of human and animal diseases such as human influenza, avian influenza, swine influenza, and equine influenza.

Clinical symptoms caused by influenza viruses include acute high fever, generalized pain, significant fatigue and respiratory symptoms. Human influenza is mainly caused by influenza A virus and influenza B virus. Influenza A virus often undergoes antigenic variation and can be further divided into subtypes such as H1N1, H3N2, H5N1, and H7N9.

### Respiratory Syncytial Virus

Respiratory syncytial virus (RSV, referred to as syncytial virus, belonging to Paramyxoviridae) is the most common pathogen causing viral pneumonia in children, and can cause interstitial pneumonia.

RSV is similar to paraflu virus. The size of virus particles is about 150 nm, which is slightly smaller than paraflu virus. It is an RNA virus.

### Flaviviridae Virus

Flaviviridae viruses are a class of RNA viruses that mainly infect mammals, including three virus genera, i.e., flavivirus, pestivirus and hepacivirus. Dengue virus (DENV) and Zika virus (ZIKV) belong to the flavivirus genus and are transmitted by mosquito vectors. Dengue virus infection can cause obvious fever and pain symptoms, and severe dengue fever symptoms also manifest as headache, nausea, vomiting, unconsciousness, and even shock. Symptoms of Zika virus (ZIKV) infection are similar to those of dengue fever and are generally mild. Hepatitis C virus (HCV) belongs to the Hepatitis C virus genus and is the pathogen of chronic hepatitis C, which can lead to liver cirrhosis and liver cancer.

### Filoviridae Virus

Filoviridae currently includes three genera: Ebolavirus, Marburgvirus, and Cuevavirus. Both Marburg virus and Ebola virus can cause severe hemorrhagic fever. After infection, people will have high fever and bleeding symptoms, which will further lead to shock, organ failure, and even death.

### Porcine Epidemic Diarrhea Virus (PEDV)

Porcine epidemic diarrhea virus (PEDV) belongs to the genus Coronavirus of the family Coronaviridae. Porcine epidemic diarrhea is an acute intestinal infectious disease of piglets and finishing pigs caused by PEDV.

After PEDV is infected through the mouth and nose, it enters the small intestine directly. Replication of PEDV can be carried out in the villous epithelium cytoplasm of the small intestine and colon. PEDV can cause diarrhea, which is osmotic diarrhea. Dehydration caused by severe diarrhea is the main cause of death in sick pigs.

### Bunya Virus

Bunya viruses are a large class of enveloped, segmented, negative-strand RNA viruses that belong to arboviruses. The family Bunyaviridae includes multiple genera (Bunyavirus, Phlebovirus, Nairovirus, Hantavirus, etc.), which can cause a variety of natural foci infectious diseases, such as renal syndrome Hemorrhagic fever, hantavirus pulmonary syndrome, sandfly fever, etc.

### Arenavirus

Arenaviruses are a type of single-stranded negative-strand RNA viruses with an envelope, shaped like sand grains, and are a small branch of viruses. At present, a variety of viruses have been found to be pathogenic to humans, such as Lassa fever virus (LASV), Junin virus (JUNV), Machupo virus (MACV), etc. These three viruses can cause hemorrhagic fever and other diseases, and severely threaten human health.

In the following examples, raw materials (cytidine A39-0, uridine B1-0, NHC, GS-441524, A2-0, C22-0) were purchased from Shanghai Haohong Biomedical Technology Co., Ltd. or Wuhu Nuowei Chemistry Co., Ltd., or were prepared according to the protocol recorded in the literature (Chemical Communications, 2020, 56, 13363-13364; Nature, 2016, 531, 381-385; Chinese Patent 202010313870.X).

### EXAMPLES

### Preparation Example 1: Synthesis of A1

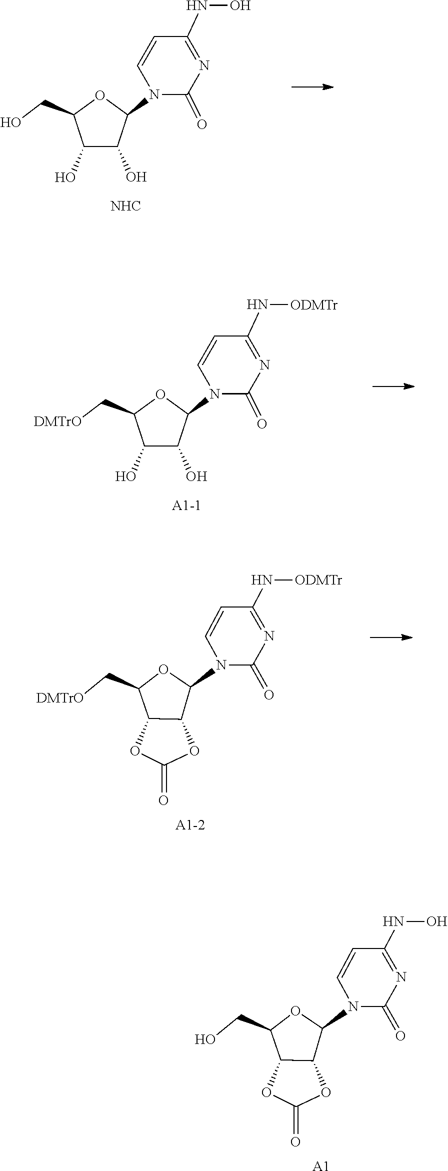

β-D-N4-hydroxycytidine (NHC) (0.97 g, 3.75 mmol) was added to dichloromethane (5 mL), and 4-dimethylaminopyridine (76 mg, 0.75 mmol), triethylamine (1.14 g, 11.25 mmol) and 4,4′-bismethoxytrityl chloride (2.80 g, 8.25 mmol) were added in sequence, and stirred at room temperature. After 6 hours, dichloromethane (20 mL) and saturated brine were added to the reaction solution, the separated organic phase was dried over anhydrous sodium sulfate, evaporated to dryness, and separated by silica gel column chromatography (petroleum ether:ethyl acetate=1:1), to obtain A1-1 as a white solid (1.81 g, yield 56%).

Carbonyldiimidazole (134.5 mg, 0.83 mmol) and A1-1 (600 mg, 0.69 mmol) were added to dichloromethane (5 mL), and stirred at room temperature. After 2 hours, the reaction solution was evaporated to dryness and separated by silica gel column chromatography (petroleum ether: ethyl acetate=2:1) to obtain A1-2 as a white foamy solid (420 mg, yield 68%).

A1-2 (420 mg, 0.47 mmol) was added to methanol (10 mL), and trifluoroacetic acid (107 mg, 0.94 mmol) was added thereto, and stirred at room temperature. After 10 minutes, a saturated sodium bicarbonate solution was added to adjust pH to neutral. The reaction solution was concentrated and separated by silica gel column chromatography (dichloromethane:methanol=15:1) to obtain a foamy solid, which was slurried in ethyl acetate to obtain Al as a white powdery solid (30 mg, yield 22%). ¹H NMR (500 MHz, DMSO-d₆) δ 10.10 (s, 1H), 9.85 (s, 1H), 6.96 (d, J=8.1 Hz, 1H), 5.88 (d, J=2.6 Hz, 1H), 5.60 (d, J=8.1 Hz, 1H), 5.50 (dd, J=7.8, 2.6 Hz, 1H), 5.21 (dd, J=7.8, 4.1 Hz, 1H), 5.14 (t, J=5.6 Hz, 1H), 4.20-4.15(m, 1H), 3.60 (t, J=5.8 Hz, 2H).

### Preparation Example 2: Synthesis of A2

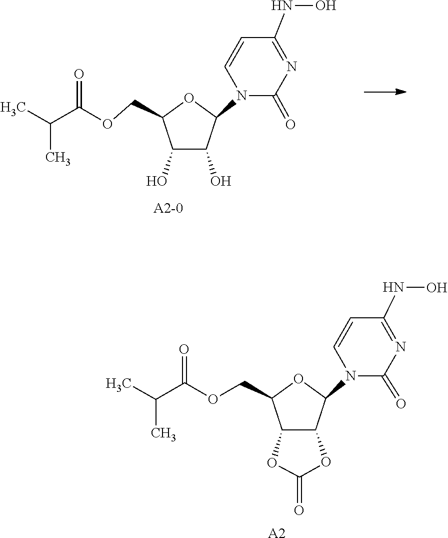

A2-0 (296 mg, 0.9 mmol) was added to dichloromethane (3 mL). Carbonyldiimidazole (225 mg, 1.4 mmol) was added under an ice bath, and after the addition was complete, it was stirred at room temperature. After the reaction of the raw materials was complete, the reaction mixture was filtered and the filter cake was washed with dichloromethane to obtain A2 as a white solid (200 mg, yield 63%). ¹H NMR (500 MHz, DMSO-d₆) δ 10.13 (s, 1H), 9.92 (d, J=2.3 Hz, 1H), 6.96 (d, J=8.1 Hz, 1H), 5.88 (d, J=2.1 Hz, 1H), 5.60 (dd, J=8.1, 2.1 Hz, 1H), 5.57 (dd, J=7.7, 2.2 Hz, 1H), 5.29 (dd, J=7.8, 4.2 Hz, 1H), 4.44-4.40 (m, 1H), 4.32 (dd, J=11.6, 5.1 Hz, 1H), 4.21 (dd, J=11.5, 6.8 Hz, 1H), 2.62-2.54 (m, 1H), 1.12-1.08 (m, 6H).

### Preparation Example 3: Synthesis of A35 and A31

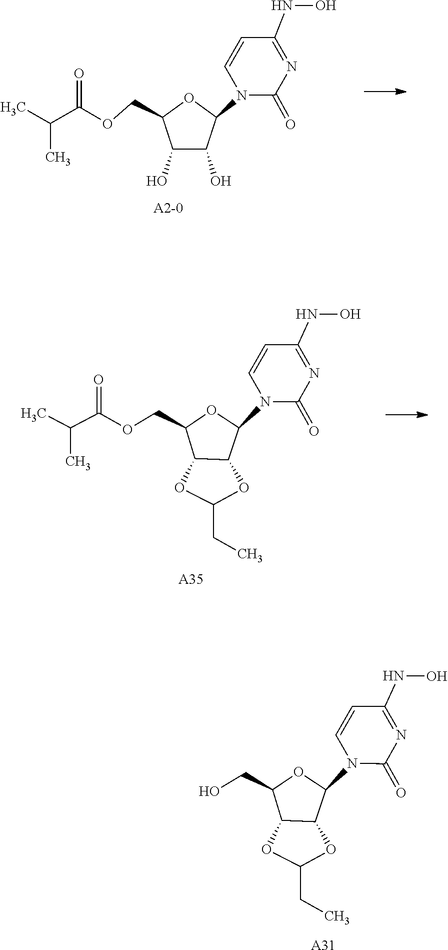

A2-0 (1.00 g, 3.04 mmol) and propionaldehyde (882 mg, 15.20 mmol) were added to dichloromethane (20 mL), and p-toluenesulfonic acid monohydrate (1.16 g, 6.08 mmol) was added slowly under an ice bath, and after the addition was completed, the reaction solution was warmed to room temperature, and stirred for 2 h. A 10% sodium carbonate aqueous solution and dichloromethane were added to the reaction solution, and the organic phase was separated. The organic phase was washed with saturated brine, dried over anhydrous sodium sulfate, and separated by silica gel column chromatography (petroleum ether: ethyl acetate=20:1 to 1:1) to obtain A35 as a white solid (897 mg, yield 80%). ¹H NMR (500 MHz, CD₃OD) δ 6.87 (d, J=8.2 Hz, 1H), 5.69 (d, J=2.3 Hz, 1H), 5.57 (d, J=8.1 Hz, 1H), 5.07 (t, J=4.5 Hz, 1H), 4.93 (dd, J=6.7, 2.3 Hz, 1H), 4.74 (dd, J=6.7, 3.7 Hz, 1H), 4.30-4.26 (m, 2H), 4.26-4.22 (m, 1H), 2.64-2.55 (m, 1H), 1.78-1.72 (m, 2H), 1.18-1.14 (m, 6H), 0.99 (t, J=7.5 Hz, 3H).

A35 (400 mg, 1.08 mmol) was added to a 7M ammonia methanol solution (15 mL), and stirred overnight at room temperature. The reaction solution was concentrated and separated by silica gel column chromatography (dichloromethane:methanol=50:1 to 15:1) to obtain A31 as a white solid (259 mg, yield 80%). ¹H NMR (600 MHz, CD₃OD) δ 7.03 (d, J=8.2 Hz, 1H), 5.81 (d, J=3.2 15 Hz, 1H), 5.58 (d, J=8.2 Hz, 1H), 5.07 (t, J=4.5 Hz, 1H), 4.81 (dd, J=6.7, 3.2 Hz, 1H), 4.71 (dd, J=6.7, 3.4 Hz, 1H), 4.15-4.12 (m, 1H), 3.75 (dd, J=11.9, 3.8 Hz, 1H), 3.71 (dd, J=11.9, 4.7 Hz, 1H), 1.79-1.73 (m, 2H), 1.00 (t, J=7.5 Hz, 3H).

### Preparation Example 4: Synthesis of A36 and A32

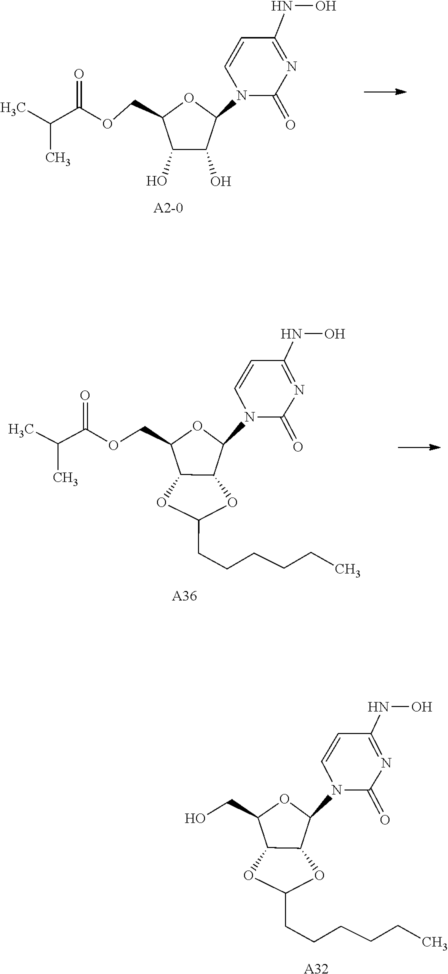

A2-0 (1.00 g, 3.04 mmol) and n-heptanal (1.73 g, 15.20 mmol) were added to dichloromethane (20 mL), and p-toluenesulfonic acid monohydrate (1.16 g, 6.08 mmol) was added slowly under an ice bath, and after the addition was completed, the reaction solution was warmed to room temperature, and stirred for 2 h. A 10% sodium carbonate aqueous solution and dichloromethane were added to the reaction solution, and the organic phase was separated. The organic phase was washed with saturated brine, dried over anhydrous sodium sulfate, and separated by silica gel column chromatography (petroleum ether:ethyl acetate=20:1 to 1:1) to obtain A36 as a white solid (1.03 g, yield 80%). ¹H NMR (500 MHz, CD₃OD) δ 6.87 (d, J=8.2 Hz, 1H), 5.68 (d, J=2.3 Hz, 1H), 5.57 (d, J=8.2 Hz, 1H), 5.09 (t, J=4.7 Hz, 1H), 4.92 (dd, J=6.7, 2.3 Hz, 1H), 4.73 (dd, J=6.7, 3.6 Hz, 1H), 4.30-4.26 (m, 2H), 4.25-4.22 (m, 1H), 2.64-2.55 (m, 1H), 1.76-1.70 (m, 30 2H), 1.49-1.41 (m, 2H), 1.39-1.27 (m, 6H), 1.18-1.13 (m, 6H), 0.91 (t, J=6.9 Hz, 3H).

A36 (600 mg, 1.41 mmol) was added to a 7M ammonia methanol solution (15 mL), and stirred overnight at room temperature. The reaction solution was concentrated and separated by silica gel column chromatography (dichloromethane:methanol=50:1 to 15:1) to obtain A32 as a white solid (375 mg, yield 75%). 1H NMR (500 MHz, CD₃OD) δ 7.04 (d, J=8.2 Hz, 1H), 5.80 (d, J=3.1 Hz, 1H), 5.58 (d, J=8.2 Hz, 1H), 5.10 (t, J=4.7 Hz, 1H), 4.80 (dd, J=6.6, 3.1 Hz, 1H), 4.70 (dd, J=6.6, 3.4 Hz, 1H), 4.15-4.11 (m, 1H), 3.75 (dd, J=11.9, 3.8 Hz, 1H), 3.70 (dd, J=11.9, 4.7 Hz, 1H), 1.77-1.70 (m, 2H), 1.51-1.42 (m, 2H), 1.40-1.28 (m, 6H), 0.91 (t, J=6.9 Hz, 3H).

### Preparation Example 5: Synthesis of A37 and A33

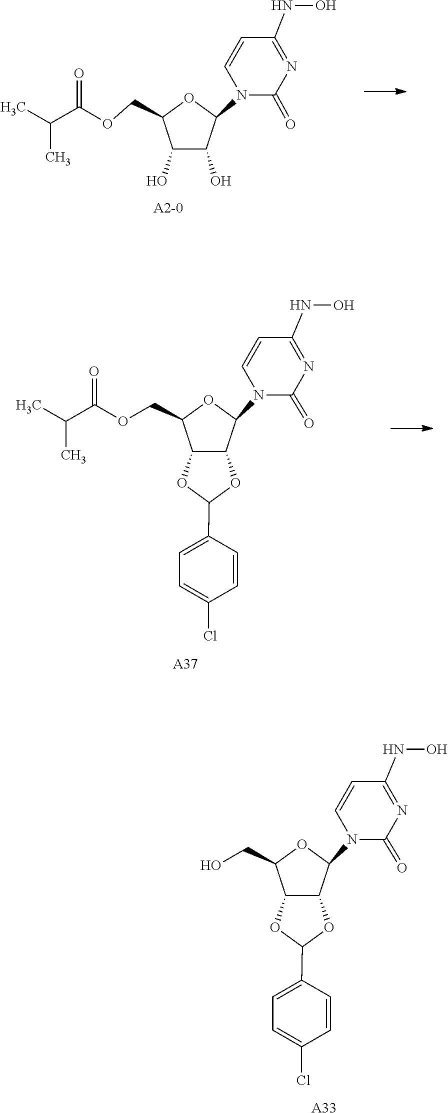

A2-0 (0.33 g, 1 mmol), anhydrous zinc chloride (0.68 g, 5 mol) and 4-chlorobenzaldehyde (1.40 g, 10 mmol) were sequentially added to anhydrous tetrahydrofuran (10 mL), and stirred at 70° C. overnight. The reaction solution was poured into a sodium carbonate aqueous solution and extracted with ethyl acetate, and the organic phase was separated. The organic phase was washed with saturated brine, dried over anhydrous sodium sulfate, concentrated, and separated by silica gel column chromatography (dichloromethane:methanol=50:1) to obtain A37 as a white solid (0.15 g, yield 33%). ¹H NMR (500 MHz, CD₃OD) δ 7.57-7.54 (m, 2H), 7.49-7.44 (m, 2H), 6.93 (d, J=8.2 Hz, 1H), 6.01 (s, 1H), 5.81 (d, J=2.3 Hz, 1H), 5.61 (d, J=8.2 Hz, 1H), 5.18 (dd, J=6.8, 2.2 Hz, 1H), 4.44-4.30 (m, 3H), 2.67-2.58 (m, 1H), 1.20-1.17 (m, 6H). ESI-MS m/z=450.0 [M−1]⁻.

A37 (0.15 g, 0.33 mmol) and potassium carbonate (0.04 g, 0.33 mmol) were added into anhydrous methanol (5 mL), and stirred at room temperature for 4 hours. The reaction solution was evaporated to dryness and added with ethyl acetate and water, and the organic phase was separated, washed with saturated brine, dried over anhydrous sodium sulfate, and separated by silica gel column chromatography (dichloromethane:methanol=30:1) to obtain A33 as a white solid (0.1 g, yield 78.8%). ¹H NMR (500 MHz, CD₃OD) δ 7.59-7.56 (m, 2H), 7.47-7.44 (m, 2H), 7.10 (d, J=8.2 Hz, 1H), 6.02 (s, 1H), 5.95 (d, J=3.1 Hz, 1H), 5.62 (d, J=8.2 Hz, 1H), 5.05 (dd, J=6.7, 3.1 Hz, 1H), 4.94 (dd, J=6.7, 3.3 Hz, 1H), 4.31-4.27 (m, 1H), 3.84-3.75 (m, 2H).

Preparation Example 6: Synthesis of A38 and A34

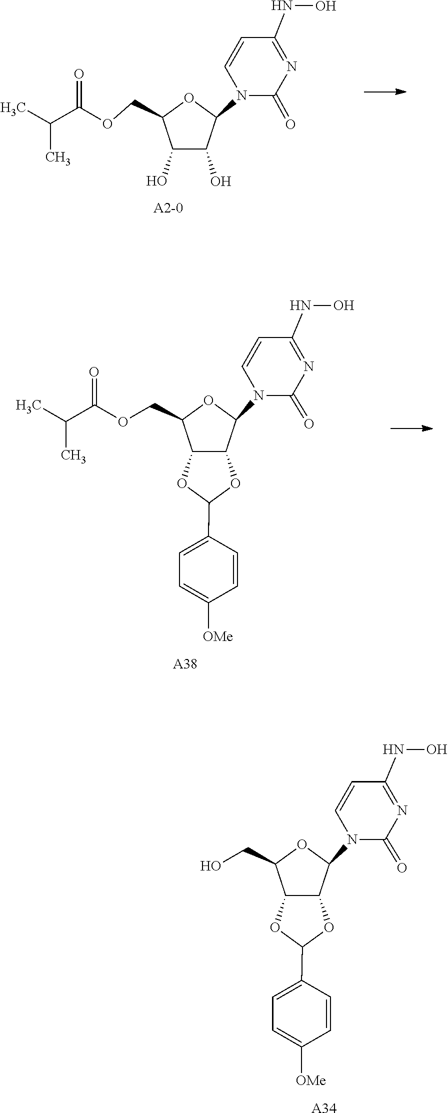

To N,N-dimethylformamide (20 mL) were sequencely added p-toluenesulfonic acid monohydrate (1.16 g, 6.08 mmol), p-methoxybenzaldehyde (2.07 g, 15.20 mmol) and 2,2-dimethoxypropane (1.58 g, 15.20 mmol) under an ice bath. After the addition, the reaction solution was warmed to room temperature and stirred for 2 hours. A2-0 (1.00 g, 3.04 mmol) was added and the stirring continued for 4 hours. The reaction solution was added to water and extracted with ethyl acetate, and the organic phase was separated, washed with saturated brine, dried over anhydrous sodium sulfate, and evaporated to dryness. The residue was sluried in a mixture of ethyl acetate and methyl tert-butyl ether to obtain A38, which is a pair of diastereomers (6:4), as a white solid (1.08 g, yield 80%). The ¹H NMR data of the main isomer is as follows: ¹H NMR (600 MHz, DMSO-d₆) δ 10.07 (s, 1H), 9.73 (d, J=2.2 Hz, 1H), 7.47-7.43 (m, 2H), 6.99-6.96 (m, 2H), 6.95 (d, J=8.1 Hz, 1H), 5.93 (s, 1H), 5.82 (d, J=2.3 Hz, 1H), 5.58 (dd, J=8.2, 1.9 Hz, 1H), 5.08 (dd, J=6.8, 2.4 Hz, 1H), 4.85 (dd, J=6.9, 3.8 Hz, 1H), 4.33-4.29 (m, 1H), 4.26 (dd, J=11.5, 4.7 Hz, 1H), 4.20 (dd, J=11.5, 6.5 Hz, 1H), 3.78 (s, 3H), 2.60-2.52 (m, 1H), 1.10-1.07 (m, 6H).

Anhydrous potassium carbonate (62 mg, 0.46 mmol) and A38 (335 mg, 0.75 mmol) were added to methanol (8 mL), stirred at 35° C. for 3 h, and adjusted the pH to neutral with a 2M dilute hydrochloric acid. The reaction solution was evaporated to dryness; saturated brine and ethyl acetate were added, and the organic phase was separated, dried over anhydrous sodium sulfate, concentrated, and separated by silica gel column chromatography (dichloromethane:methanol=20:1) to obtain A34 as a white solid (143 mg, yield 50.6%).

### Preparation Example 7: Synthesis of A39

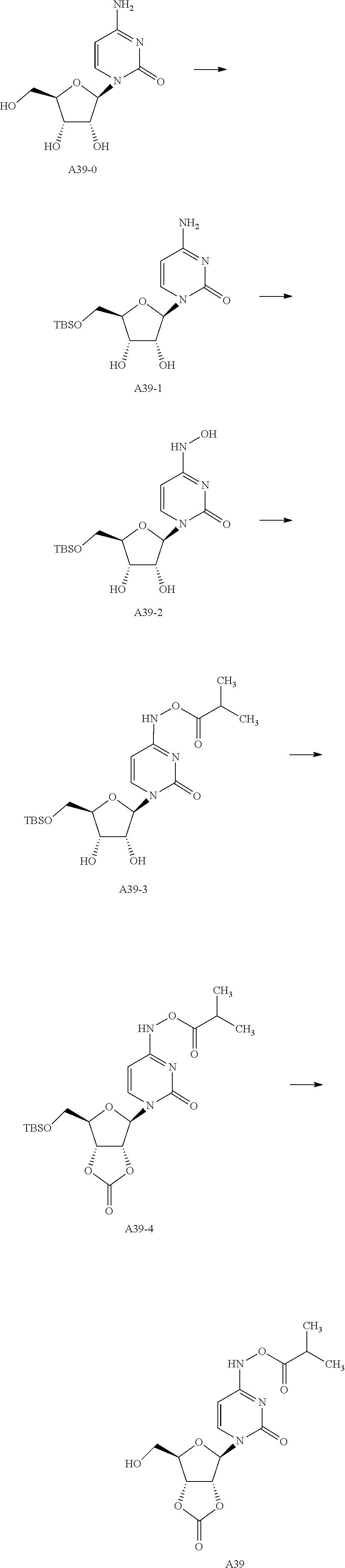

Cytidine A39-0 (1.2 g, 4.94 mmol) was added to anhydrous pyridine, and imidazole (1.34 g, 19.69 mmol) and tert-butyldimethylsilyl chloride (1.12 g, 7.41 mmol) were added sequentially under an ice bath, and stirred under the ice bath. After 2 hours, methanol (5 mL) was added to the reaction solution, and the reaction solution was evaporated to dryness, and separated by silica gel column chromatography (dichloromethane:methanol=25:1) to obtain A39-1 as an oil (1.62 g, yield 92%).

A39-1 (2.72 g, 7.61 mmol) was added into waterhydroxylamine sulfate (1.51 g, 9.14 mmol) was added therein at room temperature, and the mixture was stirred overnight at 70° C. After staying overnight, ethyl acetate was added to the reaction solution, and the organic phase was separated, washed with saturated sodium bicarbonate and saturated sodium chloride aqueous solution successively, dried over anhydrous sodium sulfate, and evaporated to dryness to obtain the crude A39-2 as a foamy solid (1.32 g, yield 47%).

A39-2 (54.6 mg, 0.147 mmol) was added to dichloromethane, and triethylamine (30 mg, 0.294 mmol) and isobutyric anhydride (24 mg, 0.147 mmol) were added successively under an ice bath, and stirred under the ice bath. After 4 hours, methanol (1 mL) was added to the reaction solution; the reaction solution was concentrated, then water was added therein, and the mixture was extracted with ethyl acetate. The organic phase was separated, washed with saturated sodium chloride, dried over anhydrous sodium sulfate, and separated by silica gel column chromatography (dichloromethane:methanol=75:1) to obtain A39-3 as a white solid (40 mg, yield 62%).

A39-3 (125 mg, 0.282 mmol) was added into dichloromethane, then carbonyldiimidazole (46 mg, 0.282 mmol) was slowly added therein, and the mixture was stirred at room temperature. After 20 15 minutes, the reaction solution was evaporated to dryness; water was added, and the mixture was extracted with ethyl acetate. The organic phase was separated, washed with saturated sodium chloride, dried over anhydrous sodium sulfate, and separated by silica gel column chromatography (ethyl acetate:petroleum ether=2:1) to obtain A39-4 as a white foamy solid (127 mg, yield 97%).

A39-4 (71 mg, 0.152 mmol) was added into tetrahydrofuran; acetic acid (4.6 mg, 0.076 mmol) and tetrabutylammonium fluoride (0.15 mL, 0.152 mmol) were added in sequence, and the mixture was stirred at room temperature. After 2 hours, water and ethyl acetate were added to the reaction solution. The organic phase was separated, washed successively with saturated sodium bicarbonate and saturated sodium chloride, dried over anhydrous sodium sulfate, and separated by silica gel column chromatography (ethyl acetate: petroleum ether:methanol=10:10:1) to obtain A39 as a white solid (45 mg, yield 83%). ¹H NMR shows that A39 has tautomers in deuterated methanol, and the ratio of the two is about 6:1. ¹H NMR (500 MHz, CD₃OD) δ 7.36-7.26 (m, 1H), 5.85 (d, J=2.2 Hz, 1H), 5.74 (d, J=8.1 Hz, 1H), 5.57 (dd, J=7.7, 2.2 Hz, 1H), 5.33 (dd, J=7.6, 3.9 Hz, 1H), 4.33 (q, J=4.8 Hz, 1H), 3.85-3.79 (m, 2H), 2.91-2.71 (m, 1H), 1.24 (d, J=6.9 Hz, 6H).

### Preparation Example 8: Synthesis of A41

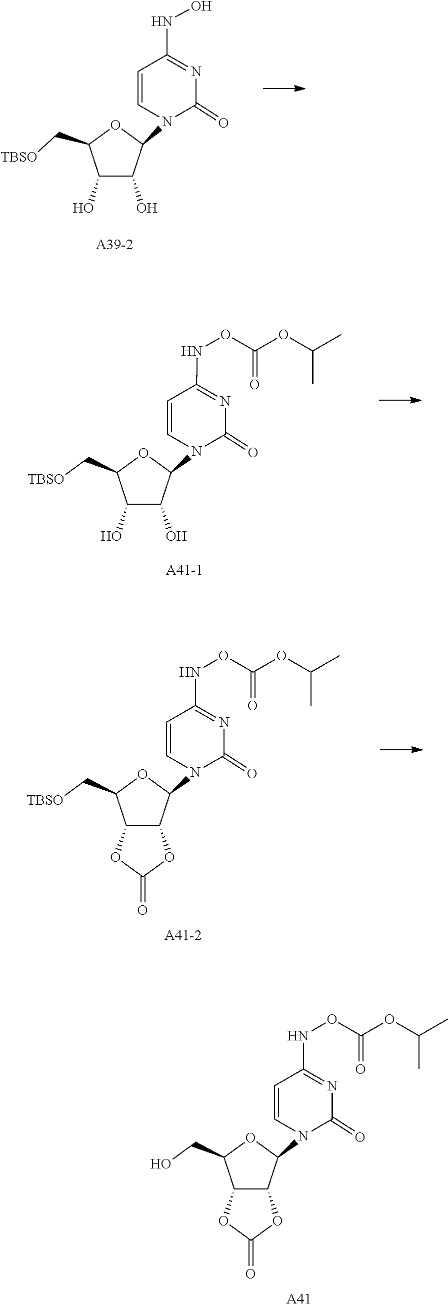 [SMILES: CC(C)(C)[Si](C)(C)OC[C@H]1O[C@@H](n2ccc(NO)nc2=O)[C@H](O)[C@@H]1O ; CC(C)OC(=O)ONc1ccn([C@@H]2O[C@H](CO[Si](C)(C)C(C)(C)C)[C@@H](O)[C@H]2O)c(=O)n1 ; CC(C)OC(=O)ONc1ccn([C@@H]2O[C@H](CO[Si](C)(C)C(C)(C)C)[C@H]3OC(=O)O[C@H]32)c(=O)n1 ; CC(C)OC(=O)ONc1ccn([C@@H]2O[C@H](CO)[C@H]3OC(=O)O[C@H]32)c(=O)n1 ; "A41"]

A39-2 (208 mg, 0.557 mmol) was added to dichloromethane, and triethylamine (113 mg, 1.114 mmol) and isopropyl chloroformate (76 mg, 0.613 mmol) were added successively under an ice bath, and stirred under the ice bath. After 4 hours, methanol (1 mL) was added to the reaction solution. The reaction solution was concentrated, then water was added therein, and the mixture was extracted with ethyl acetate. The organic phase was separated, washed with saturated sodium chloride, dried over anhydrous sodium sulfate, and separated by silica gel column chromatography (dichloromethane:methanol=75:1) to obtain A41-1 as a white foamy solid (148 mg, yield 58%).

A41-1 (275 mg, 0.599 mmol) was added into dichloromethane, then carbonyldiimidazole (146 mg, 0.899 mmol) was slowly added therein, and the mixture was stirred at room temperature. After minutes, the reaction solution was evaporated to dryness, water was added, and the mixture was extracted with ethyl acetate. The organic phase was separated, washed with saturated sodium chloride, dried over anhydrous sodium sulfate, and separated by silica gel column chromatography (ethyl acetate:petroleum ether=2:1) to obtain A41-2 as a white powdery solid (233 mg, yield 80%).

A41-2 (233 mg, 0.481 mmol) was added into tetrahydrofuran, acetic acid (15 mg, 0.241 mmol) and tetrabutylammonium fluoride (0.48 mL, 0.152 mmol) were added in sequence, and the mixture was stirred at room temperature. After 2 hours, water and ethyl acetate were added to the reaction solution, the organic phase was separated, washed successively with saturated sodium bicarbonate and saturated sodium chloride, dried over anhydrous sodium sulfate, and separated by silica gel column chromatography (ethyl acetate: petroleum ether:methanol=10:10:1) to obtain A41 as a white solid (112 mg, yield 63%). ¹H NMR shows that A41 has tautomers in deuterated methanol, and the ratio of the two is about 7:1. ¹H NMR (500 MHz, CD₃OD) δ 7.34-7.24 (m, 1H), 5.83 (d, J=2.1 Hz, 1H), 5.72 (d, J=8.1 Hz, 1H), 5.56 (dd, J=7.6, 2.2 Hz, 1H), 5.32 (dd, J=7.6, 3.9 Hz, 1H), 5.02-4.94 (m, 1H), 4.32 (q, J=4.9 Hz, 1H), 3.84-3.78 (m, 2H), 1.36 (d, J=6.2 Hz, 6H).

### Preparation Example 9: Synthesis of A52

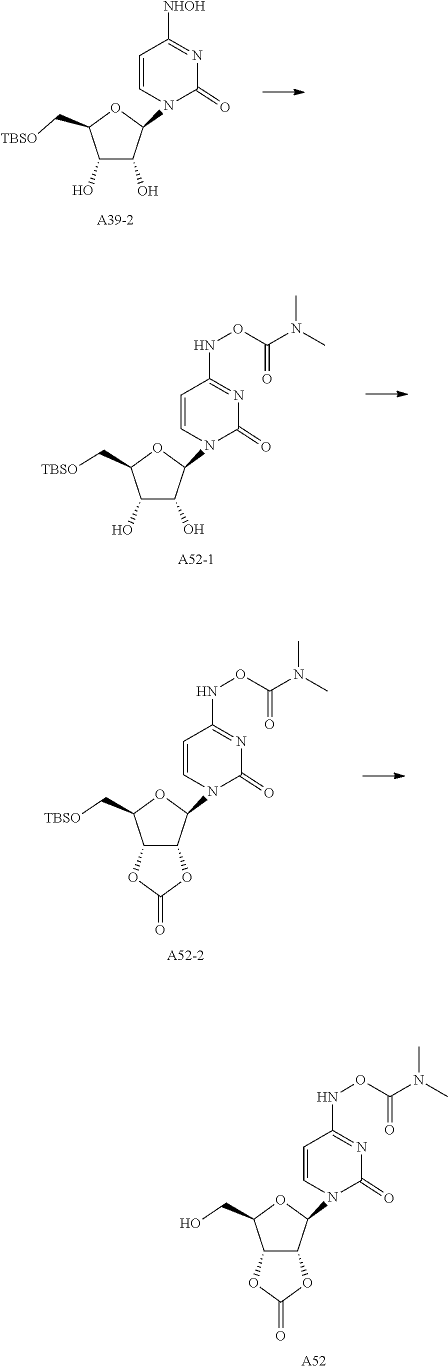

30

A39-2 (373 mg, 1.0 mmol) was added into pyridine (10 mL), then dimethylcarbamoyl chloride (113 mg, 1.114 mmol) was added under an ice bath, and the mixture was reacted overnight at room temperature. The reaction solution was evaporated to dryness, water was added, and the mixture was extracted with ethyl acetate. The organic phase was separated, washed with saturated sodium chloride, dried over anhydrous sodium sulfate, and separated by silica gel column chromatography (dichloromethane:methanol=60:1) to obtain A52-1 as a white foamy solid (186 mg, yield 42%).

Referring to the reaction conditions of the second and third steps in Example 8, using A52-1 (186 mg, 0.42 mmol) as a raw material, A52 was prepared as a foamy solid (60 mg, yield 40%).

Preparation Example 10: Synthesis of B1

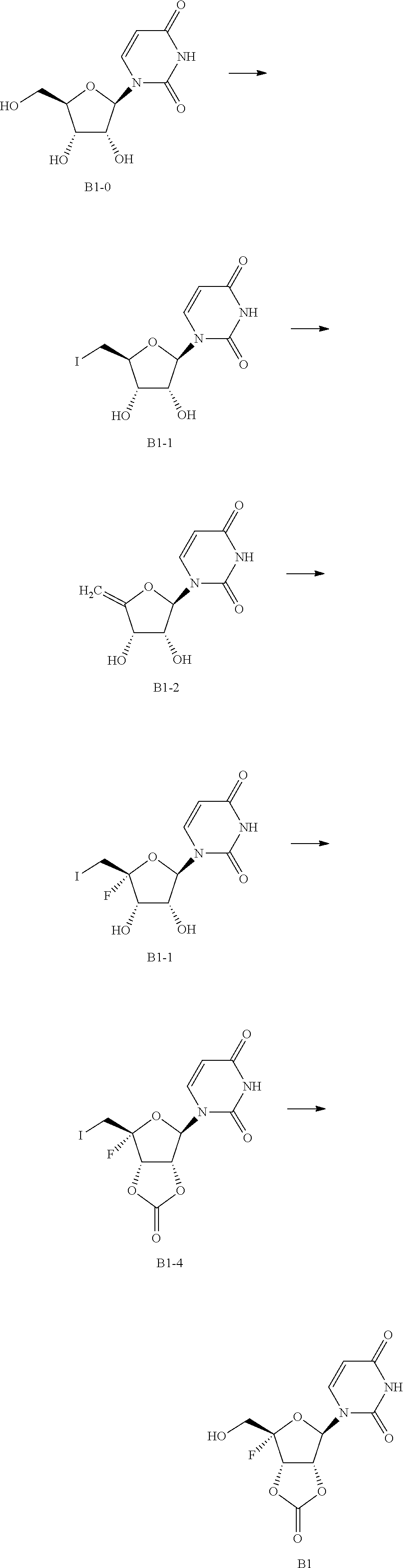

Triphenylphosphine (11.79 g, 45 mmol) was added to pyridine (50 mL), and iodine (11.42 g, 45 mmol) was added under an ice bath, stirred for 10 minutes, and then warmed to room temperature. Uridine B1-0 (7.32 g, 20 mmol) was added, and stirred overnight at 25° C. Saturated sodium thiosulfate and saturated sodium bicarbonate were added in sequence, and the reaction solution was rotary evaporated to dryness. Tetrahydrofuran and saturated saline were added to the concentrate, and the organic phase and the aqueous phase were separated. The aqueous phase was extracted twice with tetrahydrofuran, and the organic phases were combined, dried over anhydrous sodium sulfate, concentrated, and separated by silica gel column chromatography (dichloromethane:methanol=15:1) to obtain B1-1 as a yellow solid (5.30 g, yield 75%).

Sodium (1.04 g, 45 mmol) was added to methanol (40 mL) under an ice bath to prepare a solution of sodium methoxide in methanol. B1-1 (5.30 g, 15 mmol) was added into methanol (50 mL), the solution of sodium methoxide in methanol prepared above was added, and the mixture was stirred under reflux at 67° C. After 2 h, a dilute hydrochloric acid was added to adjust pH to 8-9, and the reaction solution was rotary evaporated to dryness, and separated by silica gel column chromatography (dichloromethane:methanol=15:1) to obtain B1-2 as a white solid (2.1 g, yield 62%).

B1-2 (2.10 g, 9.3 mmol) and triethylamine trihydrofluoride (1.80 g, 11.2 mmol) were added to acetonitrile (30 mL), and N-iodosuccinimide (2.51 g, 11.2 mmol) was added in batches under an ice bath, stirred for 30 minutes, naturally settled for 1 hour, filtered, rinsed with dichloromethane to obtain B1-3 (1.5 g, yield 43%).

B1-3 (950 mg, 2.55 mmol) was added to anhydrous tetrahydrofuran (5 mL), then carbonyldiimidazole (616 mg, 3.8 mmol) was added therein, and the mixture was stirred at room temperature for 30 min. The reaction solution was evaporated to dryness, and separated by silica gel column chromatography (petroleum ether:ethyl acetate=1:1 to 1:2) to obtain B1-4 as a white foamy solid (520 mg, yield 51%).

Trifluoroacetic acid was added to an aqueous solution of tetrabutylammonium hydroxide (1.45 g, 5.5 mmol) to adjust pH to about 4, and the above solution was added to a solution of B1-4 (440 mg, 1.1 mmol) in dichloromethane (5 mL); m-chloroperoxybenzoic acid (952 mg, 5.5 mmol) was added therein, and the mixture was stirred at room temperature. After 7 h, saturated sodium thiosulfate solution was added, and then saturated brine and ethyl acetate were added. The ethyl acetate layer and the water layer were separated. Then the aqueous layer was extracted with tetrahydrofuran, and the tetrahydrofuran layer was separated. The organic phase was combined, dried with anhydrous sodium sulfate, concentrated, separated by silica gel column chromatography (petroleum ether:ethyl acetate=1:3), and slurried in ethyl acetate to obtain B1 as a white solid (220 mg, yield 69%). 1 H NMR (500 MHz, DMSO-d₆) δ 11.62 (s, 1H), 7.80 (d, J=8.1 Hz, 1H), 6.29 (d, J=1.3 Hz, 1H), 5.75 (dd, J=7.3, 1.3 Hz, 1H), 5.71 (d, J=8.1 Hz, 1H), 5.61 (t, J=6.6 Hz, 1H), 5.55 (dd, J=12.4, 7.3 Hz, 1H), 3.78-3.69 (m, 1H), 3.67-3.58 (m, 1H).

### Preparation Example 11: Synthesis of B2

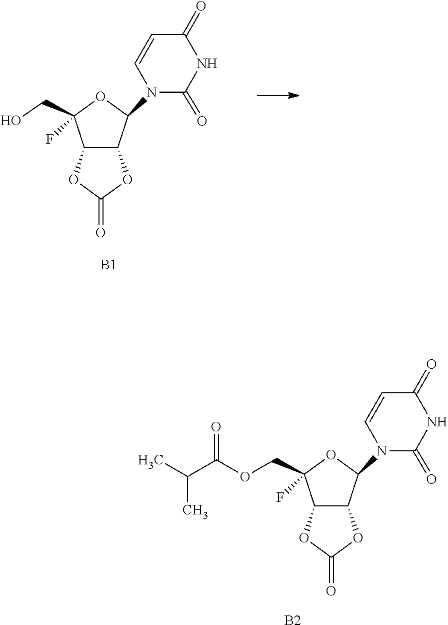

B1 (57 mg, 0.2 mmol), triethylamine (81 mg, 0.8 mmol) and 4-dimethylaminopyridine (12 mg, 0.1 mmol) were sequentially added to dichloromethane (3 mL), isobutyryl chloride (32 mg, 0.3 mmol) was added therein, and the mixture was stirred at room temperature. After 2 hours, dichloromethane and saturated saline were added, and the organic phase was separated, dried over anhydrous sodium sulfate, concentrated, and subjected to silica gel column chromatography (dichloromethane:methanol=30:1) to obtain B2 as a white solid (54 mg, yield 75%). ¹H NMR (400 MHz, DMSO-d₆) δ 11.63 (s, 1H), 7.79 (d, J=8.0 Hz, 1H), 6.35 (s, 1H), 5.77 (d, J=7.3 Hz, 1H), 5.71 (d, J=8.0 Hz, 1H), 5.62 (dd, J=11.7, 7.3 Hz, 1H), 4.47-4.35 (m, 2H), 2.66-2.57 (m, 1H), 1.11 (d, J=7.0 Hz, 6H).

### Preparation Example 12: Synthesis of C1

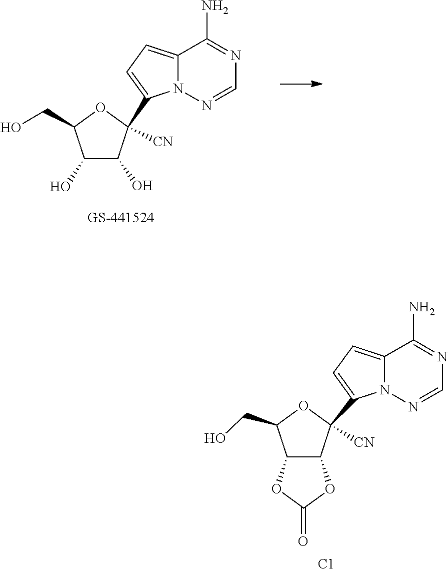

GS-441524 (85 mg, 0.292 mmol) was added to N,N-dimethylformamide, then carbonyldiimidazole (48 mg, 0.292 mmol) was added therein, and the mixture was stirred at room temperature. After 15 minutes, water and ethyl acetate were added to the reaction solution, and the organic phase was separated, washed with saturated brine, dried over anhydrous sodium sulfate, and separated by silica gel column chromatography (dichloromethane:methanol=20:1) to obtain Cl as a white solid (19 mg, yield 21%). ¹H NMR (500 MHz, DMSO-d₆) δ 8.16 (s, 1H), 8.02 (s, 1H), 8.00 (s, 1H), 7.02-6.98 (m, 2H), 5.96 (d, J=7.9 Hz, 1H), 5.40 (dd, J=7.8, 4.0 Hz, 1H), 5.28 (t, J=5.7 Hz, 1H), 4.50 (q, J=4.7 Hz, 1H), 3.72-3.65 (m, 1H), 3.64-3.58 (m, 1H).

### Preparation Example 13: Synthesis of C22

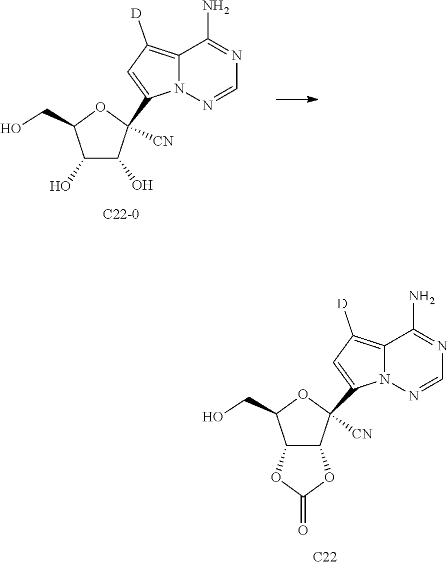 [SMILES: [2H]c1cc([C@]2(C#N)O[C@H](CO)[C@@H](O)[C@H]2O)n2ncnc(N)c12 ; [2H]c1cc([C@]2(C#N)O[C@H](CO)[C@H]3OC(=O)O[C@H]32)n2ncnc(N)c12 ; "C22"]

C22-0 (48.7 mg, 0.17 mmol) was added to N,N-dimethylformamide (1 mL), then carbonyldiimidazole (28 mg, 0.17 mmol) was added therein, and the mixture was stirred at room temperature. After 5 minutes, water and ethyl acetate were added, and the aqueous layer and the ethyl acetate layer were separated. The aqueous layer was extracted three times with ethyl acetate, and the organic phases were combined, dried over anhydrous sodium sulfate, concentrated, and purified by silica gel column chromatography (dichloromethane:methanol=20:1) to obtain 18 mg of C22 as a white solid. ¹H NMR (500 MHz, DMSO-d₆) δ 8.12 (s, 1H), 8.02 (s, 1H), 8.00 (s, 1H), 6.99 (s, 1H), 5.95 (d, J=7.8 Hz, 1H), 5.39 (dd, J=7.8, 4.0 Hz, 1H), 5.25 (t, J=5.7 Hz, 1H), 4.52-4.48 (m, 1H), 3.71-3.66 (m, 1H), 3.64-3.58 (m, 1H).

### Preparation Example 14: Synthesis of C38

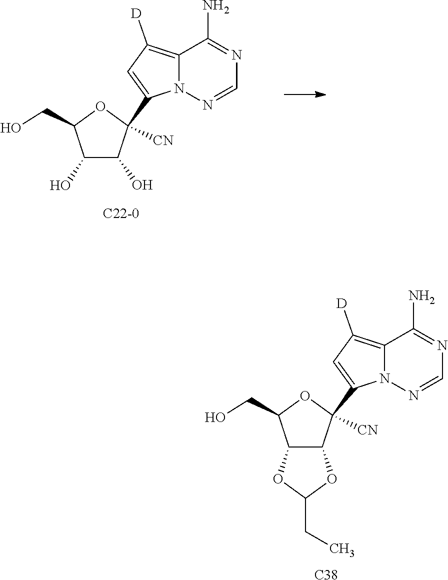

Propionaldehyde (290 mg, 5.0 mmol) and C22-0 (292 mg, 1.0 mmol) were added to dichloromethane (10 mL), then p-toluenesulfonic acid monohydrate (380 mg, 2.0 mmol) was added therein under an ice bath, the mixture was stirred for 10 minutes, warmed to room temperature, and continued to stir for 2 hours. The reaction solution was poured into saturated sodium bicarbonate solution and extracted with dichloromethane. An ammonia ethanol solution was added to the organic layer and concentrated to obtain an oil. Ethyl acetate and saturated brine were added to the concentrate, and the organic phase was seperated, dried over anhydrous sodium sulfate, and separated by silica gel column chromatography (dichloromethane:methanol=50:1 to 15:1) to obtain C38 as a white solid (96 mg, yield 30%). ¹H NMR (500 MHz, DMSO-d₆) δ 8.05-7.88 (m, 3H), 6.91 (s, 1H), 5.35 (d, J=6.7 Hz, 1H), 5.17 (t, J=5.0 Hz, 1H), 5.03 (t, J=5.6 Hz, 1H), 4.80 (dd, J=6.7, 2.9 Hz, 1H), 4.38-4.35 (m, 1H), 3.59-3.48 (m, 2H), 1.90-1.83 (m, 2H), 0.99 (t, J=7.5 Hz, 3H).

### Preparation Example 15: Synthesis of A54

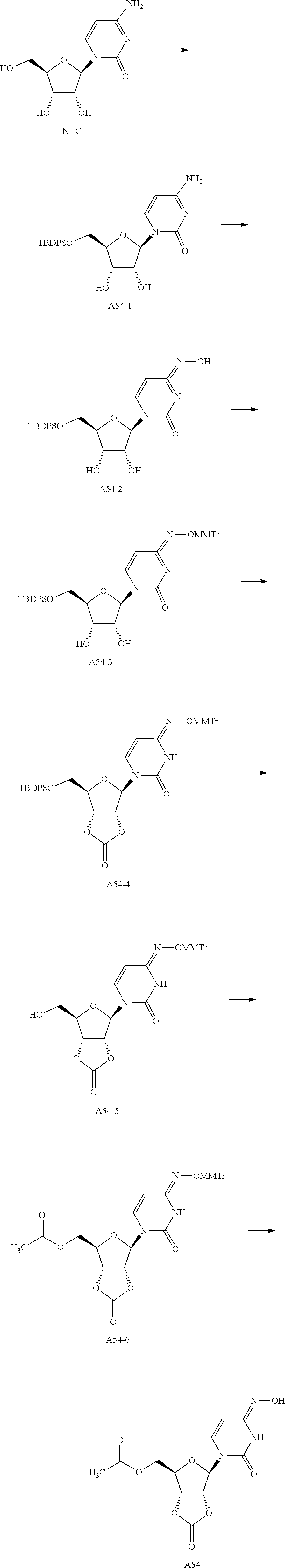

NHC (15.0 g, 61.67 mmol), imidazole (12.6 g, 185.29 mmol) were added to N,N-dimethylformamide (50 mL), and tert-butyldiphenylchlorosilane (25.4 g, 92.51 mmol) was added dropwise under an ice bath. After the addition was completed, the reaction solution was warmed to room temperature and stirred. After 4 hours, distilled water (100 mL) was added dropwise to the reaction solution to precipitate a solid, which was filtered and the filter cake was air-dried at 50° C. to obtain A54-1 as a white solid (25 g, yield 84%).

A54-1 (25.0 g, 51.90 mmol), hydroxylamine sulfate (25.0 g, 152.44 mmol) were added into acetonitrile/water (120 mL/120 mL). After the addition was complete, the reaction solution was heated to 60° C. and stirred overnight. After the reaction was complete, the reaction solution was cooled to room temperature, and the organic layer was separated. The aqueous layer was extracted with ethyl acetate, and the organic layers were combined, washed with saturated brine, dried over anhydrous sodium sulfate, and concentrated to obtain A54-2 as a white solid, which was directly used in the next reaction.

The product obtained in the previous step, 4-methoxytriphenylchloromethane (19.2 g, 62.30 mmol), triethylamine (10.5 g, 103.80 mmol) were added into dichloromethane (200 mL), and stirred at room temperature. After 2-3 hours, methanol (1 mL) was added to the reaction solution and concentrated to obtain A54-3 as a yellow foamy solid, which was directly used for the next reaction. The product obtained in the previous step was added into dichloromethane (200 mL), then carbonyldiimidazole (10.1 g, 62.28 mmol) was added under an ice bath, and the mixture was stirred at room temperature after the addition was complete. After 2 hours, the reaction solution was poured into water, extracted with dichloromethane, the organic layers were combined, dried over anhydrous sodium sulfate, concentrated, and separated by silica gel column chromatography (dichloromethane: methanol=30:1) to obtain A54-4 as an off-white solid (31.5 g, yield 76% over three steps).

A54-4 (31.5 g, 39.57 mmol), acetic acid (1.2 g, 19.79 mmol) were added to tetrahydrofuran (200 mL), then a 1M tetrabutylammonium fluoride solution (43.5 mL, 43.5 mmol) in tetrahydrofuran was added therein at room temperature, and the mixture was stirred at room temperature. After 2 hours, the reaction solution was concentrated, water and ethyl acetate were added, and the organic phase was separated, washed successively with saturated brine, dried over anhydrous sodium sulfate, concentrated, and separated by silica gel column chromatography (dichloromethane: methanol=30:1) to obtain A54-5 as an off-white solid (15 g, yield 68%).

A54-5 (800 mg, 1.43 mmol), triethylamine (289 mg, 2.86 mmol), 4-dimethylaminopyridine (35 mg, 0.29 mmol), acetic anhydride (220 mg, 2.15 mmol) were sequentially added to dichloromethane (10 mL), and stirred at room temperature. After 2 hours, dichloromethane and water were added to the reaction solution, and the organic phase was separated, washed with saturated brine, dried over anhydrous sodium sulfate, and concentrated to obtain A54-6, which was directly used in the next reaction.

The product obtained in the previous step was added to methanol (5 mL), then trifluoroacetic acid (326 mg, 2.86 mmol) was added therein, and the mixture was stirred at room temperature. After 1 hour, the reaction solution was concentrated and separated by silica gel column chromatography (dichloromethane:methanol=30:1) to obtain A54 as a white solid (40 mg, yield 9%). ¹H NMR (500 MHz, DMSO-d₆) δ 10.10 (s, 1H), 9.88 (s, 1H), 6.95 (d, J=8.1 Hz, 1H), 5.86 (d, J=2.1 Hz, 1H), 5.59 (d, J=8.1 Hz, 1H), 5.56 (dd, J=7.7, 2.2 Hz, 1H), 5.27 (dd, J=7.7, 4.2 Hz, 1H), 4.43-4.37 (m, 1H), 4.31 (dd, J=11.6, 4.7 Hz, 1H), 4.18 (dd, J=11.6, 7.0 Hz, 1H), 2.04 (s, 3H). MS m/z=328.2 [M+1]⁺.

### Preparation Example 16: Synthesis of A55

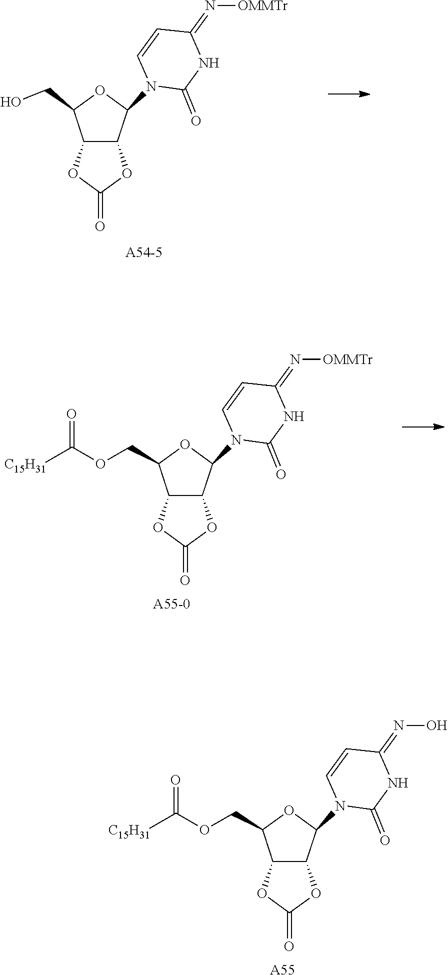

A54-5 (800 mg, 1.43 mmol), palmitic acid (554 mg, 2.16 mmol), 1-hydroxybenzotriazole (350 mg, 2.59 mmol), 1-(3-dimethylaminopropyl)-3-ethylcarbodiimide hydrochloride (663 mg, 3.46 mmol), 4-dimethylaminopyridine (176 mg, 1.44 mmol) were added to dichloromethane (10 mL), and stirred at room temperature. After 2 hours, dichloromethane and water were added to the reaction solution, and the organic phase was separated, washed with saturated brine, dried over anhydrous sodium sulfate, and concentrated to obtain A55-0, which was directly used in the next reaction.

A55-0 and trifluoroacetic acid (328 mg, 2.88 mmol) were added into methanol (5 mL), and stirred at room temperature. After 1 hour, the reaction solution was concentrated and separated by silica gel column chromatography (dichloromethane:methanol=30:1) to obtain A55 as a white solid (70 mg, yield 9%). ¹H NMR (500 MHz, DMSO-d₆) δ 10.11 (s, 1H), 9.88 (s, 1H), 6.95 (d, J=8.1 Hz, 1H), 5.86 (d, J=2.2 Hz, 1H), 5.58 (d, J=8.1 Hz, 1H), 5.55 (dd, J=7.7, 2.2 Hz, 1H), 5.26 (dd, J=7.8, 4.1 Hz, 1H), 4.42-4.36 (m, 1H),4.31 (dd, J=11.5, 4.9 Hz, 1H), 4.19 (dd, J=11.6, 7.0 Hz, 1H), 2.31 (t, J=7.4 Hz, 2H), 1.31-1.14 (m, 27H), 0.85 (t, J=6.8 Hz, 3H). MS m/z=524.5 [M+1]⁺.

### Preparation Example 17: Synthesis of A56

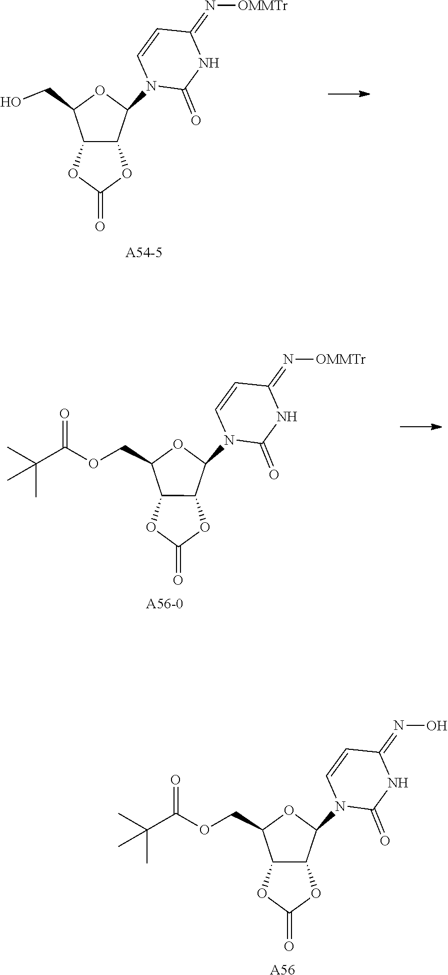

A54-5 (600 mg, 1.08 mmol), triethylamine (216 mg, 2.16 mmol), 4-dimethylaminopyridine (27 mg, 0.22 mmol), pivaloyl chloride (195 mg, 1.62 mmol) were added to dichloromethane (10 mL), and stirred at room temperature. After 2 hours, water and dichloromethane were added, and the organic phase was separated, washed with saturated brine, dried over anhydrous sodium sulfate, and concentrated to obtain A56-0, which was directly used in the next reaction.

A56-0 and trifluoroacetic acid (246 mg, 2.16 mmol) were added into methanol (5 mL), and stirred at room temperature. After 1 hour, the reaction solution was concentrated and separated by silica gel column chromatography (dichloromethane:methanol=30:1) to obtain A56 as a white solid (140 mg, yield 35%). 1 H NMR (500 MHz, DMSO-d₆) δ 10.10 (s, 1H), 9.88 (d, J=2.0 Hz, 1H), 6.94 (d, J=8.1 Hz, 1H), 5.86 (d, J=2.0 Hz, 1H), 5.61-5.54 (m, 2H), 5.27 (dd, J=7.7, 4.2 Hz, 1H), 4.40 (q, J=5.6 Hz, 1H), 4.31-4.18 (m, 2H), 1.15 (s, 9H). MS m/z=370.0 [M+1]⁺.

### Preparation Example 18: Synthesis of A4

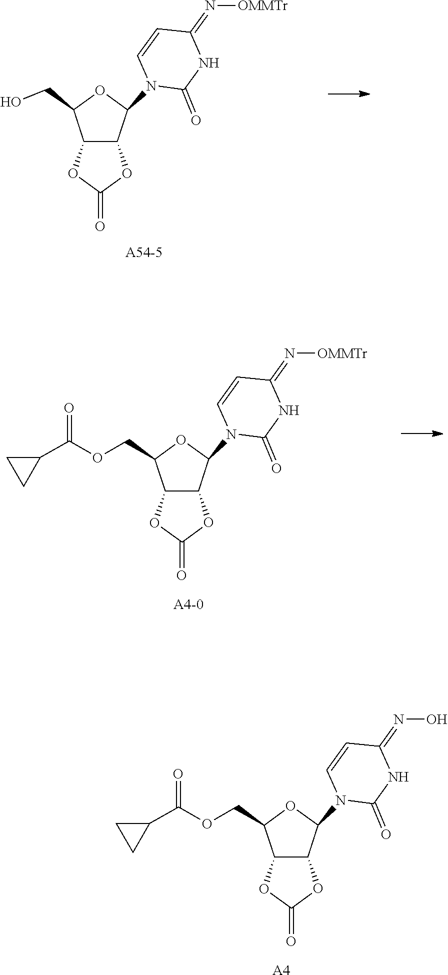

A54-5 (700 mg, 1.26 mmol), triethylamine (255 mg, 2.52 mmol), 4-dimethylaminopyridine (31 mg, 0.25 mmol), cyclopropylformyl chloride (198 mg, 1.89 mmol) were added to dichloromethane (10 mL), and stirred at room temperature. After 2 hours, water and dichloromethane were added, and the organic phase was separated, washed with saturated brine, dried over anhydrous sodium sulfate, and concentrated to obtain A4-0, which was directly used in the next reaction.

A4-0 and trifluoroacetic acid (287 mg, 2.52 mmol) were added into methanol (5 mL), and stirred at room temperature. After 1 hour, the reaction solution was concentrated and separated by silica gel column chromatography (dichloromethane:methanol=30:1) to obtain A4 as a white solid (150 mg, yield 34%). ¹H NMR (500 MHz, DMSO-d₆) δ 10.10 (s, 1H), 9.87 (d, J=2.1 Hz, 1H), 6.95 (d, J=8.1 Hz, 1H), 5.87 (d, J=2.1 Hz, 1H), 5.59 (dd, J=8.1, 2.0 Hz, 1H), 5.56 (dd, J=7.7, 2.2 Hz, 1H), 5.27 (dd, J=7.7, 4.2 Hz, 1H), 4.44-4.38 (m, 1H), 4.32 (dd, J=11.6, 4.9 Hz, 1H), 4.20 (dd, J=11.6, 7.0 Hz, 1H), 1.69-1.61 (m, 1H), 0.93-0.88 (m, 2H), 0.87-0.82 (m, 2H). MS m/z=354.2 [M+1]⁺.

### Preparation Example 19: Synthesis of A6

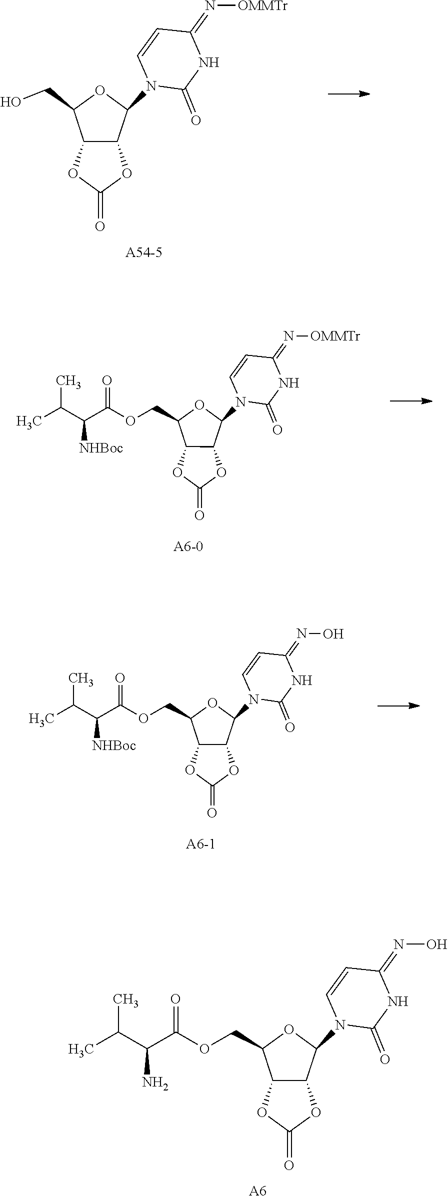

A54-5 (15.27 g, 27.39 mmol) was added to dichloromethane (200 mL), and triethylamine (11.09 g, 109.56 mmol), Boc-L-valine (8.34 g, 38.35 mmol), 1-hydroxybenzotriazole (5.56 g, 41.09 mmol), 1-(3-dimethylaminopropyl)-3-ethylcarbodiimide hydrochloride (11.56 g, 60.26 mmol), 4-dimethylaminopyridine (3.35 g, 27.39 mmol) were sequencely added under an ice bath. After the addition, the reaction solution was warmed to room temperature and stirred. After 3 hours, the reaction solution was concentrated, and water and ethyl acetate were added. The organic phase was separated, washed with saturated sodium bicarbonate and saturated brine successively, dried over anhydrous sodium sulfate, and concentrated to obtain A6-0.

A6-0 (20.73 g, 27.39 mmol) was added to dichloromethane/methanol (10:1), then trifluoroacetic acid (6.37 g, 55.82 mmol) was added therein, and the mixture was stirred at room temperature for 4 hours. The reaction solution was concentrated, and separated by silica gel column chromatography (dichloromethane:methanol=40:1) to obtain A6-1.

A6-1 (5.81 g, 12.00 mmol) was added to tetrahydrofuran (50 mL), then concentrated hydrochloric acid (12 mL, 144.00 mmol) was added therein, and the mixture was stirred overnight at room temperature. The reaction solution was concentrated, slurried with isopropanol, and filtered to obtain A6 hydrochloride as a white solid (3.4 g, yield 29% over three steps). ¹H NMR (500 MHz, CD₃OD) δ 7.87 (d, J=7.9 Hz, 1H), 6.08 (d, J=7.9 Hz, 1H), 5.96 (d, J=1.2 Hz, 1H), 5.81 (dd, J=7.4, 1.3 Hz, 1H), 5.49 (dd, J=7.5, 3.8 Hz, 1H), 4.75 (dd, J=11.5, 7.6 Hz, 1H), 4.64 (dt, J=7.8, 4.2 Hz, 1H), 4.54 (dd, J=11.6, 4.5 Hz, 1H), 4.04 (d, J=4.4 Hz, 1H), 2.41-2.26 (m, 1H), 1.10 (dd, J=6.9, 4.5 Hz, 6H).

### Preparation Example 20: Synthesis of B4

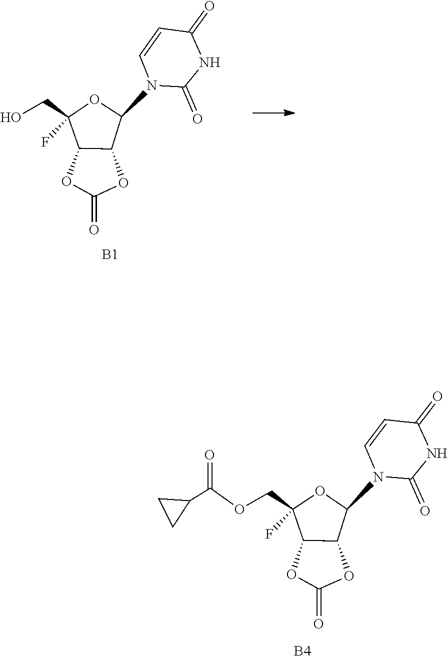

B1 (100.0 mg, 0.35 mmol) was added to dichloromethane (6 mL), and cyclopropanecarboxylic acid (42.0 mg, 0.49 mmol), 1-hydroxybenzotriazole (112.4 mg, 0.53 mmol), 1-(3-dimethylaminopropyl)-3-ethylcarbodiimide hydrochloride (147.8 mg, 0.77 mmol), 4-dimethylaminopyridine (170.8 mg, 1.40 mmol) were sequencely added under an ice bath. After the addition, the reaction solution was warmed to room temperature and stirred. After 3 hours, the reaction solution was concentrated; water and ethyl acetate were added, and the organic phase was separated, washed successively with dilute hydrochloric acid, saturated sodium bicarbonate, and saturated brine, dried over anhydrous sodium sulfate, and separated by silica gel column chromatography (petroleum ether:acetone=1:1) to obtain B4 as a white solid (95 mg, yield 80%). ¹H NMR (500 MHz, DMSO-d₆) δ 11.64 (s, 1H), 7.79 (d, J=8.0 Hz, 1H), 6.35 (d, J=1.2 Hz, 1H), 30 5.78 (dd, J=7.2, 1.2 Hz, 1H), 5.72 (dd, J=8.0, 2.2 Hz, 1H), 5.64 (dd, J=11.7, 7.3 Hz, 1H), 4.47 -4.35 (m, 2H), 1.73-1.67 (m, 1H), 0.99-0.88 (m, 4H).

### Preparation Example 21: Synthesis of B41

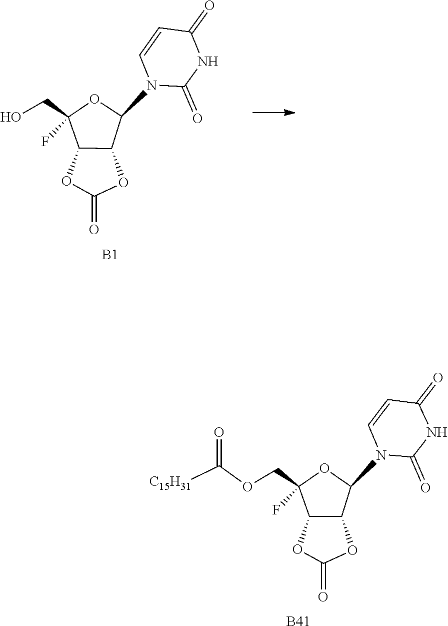

B1 (60.0 mg, 0.21 mmol) was added to dichloromethane (4 mL), and palmitic acid (74.5 mg, 0.29 mmol), 1-hydroxybenzotriazole (67.8 mg, 0.32 mmol), 1-(3-dimethylaminopropyl)-3-ethylcarbodiimide hydrochloride (88.3 mg, 0.46 mmol), 4-dimethylaminopyridine (102.5 mg, 0.84 mmol) were sequencely added under an ice bath. After the addition, the reaction solution was warmed to room temperature and stirred. After 3 hours, the reaction solution was concentrated;water and ethyl acetate were added, and the organic phase was separated, sequencely washed with dilute hydrochloric acid, saturated sodium bicarbonate, and saturated brine, dried over anhydrous sodium sulfate, and separated by silica gel column chromatography (dichloromethane:methanol=30:1) to obtain B41 as a white solid (90 mg, yield 82%). ¹H NMR (400 MHz, DMSO-d₆) δ 11.63 (s, 1H), 7.78 (d, J=8.1 Hz, 1H), 6.34 (s, 1H), 5.77 (d, J=7.5 Hz, 1H), 5.70 (d, J=7.9 Hz, 1H), 5.62 (t, J=9.7 Hz, 1H), 4.48-4.31 (m, 2H), 2.38-2.32 (m, 2H), 1.56-1.48 (m, 2H), 1.31-1.21 (m, 24H), 0.85 (t, J=6.6 Hz, 3H).

### Preparation Example 22: Synthesis of B6

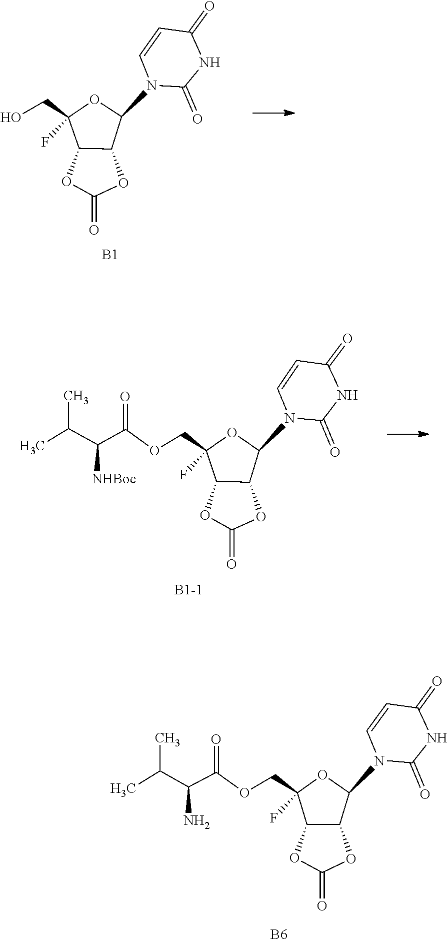

B1 (200.0 mg, 0.695 mmol) was added to dichloromethane (10 mL), and Boc-L-valine (211.0 mg, 0.973 mmol), 1-hydroxybenzotriazole (221.1 mg, 1.043 mmol), 1-(3-dimethylaminopropyl)-3-ethylcarbodiimide hydrochloride (293.6 mg, 1.529 mmol), 4-dimethylaminopyridine (339.2 mg, 2.780 mmol) were sequencely added under an ice bath. After the addition, the reaction solution was warmed to room temperature and stirred. After 3 hours, the reaction solution was concentrated; water and ethyl acetate were added, and the organic phase was separated, washed successively with dilute hydrochloric acid, saturated sodium bicarbonate, saturated brine, and dried over anhydrous sodium sulfate, and concentrated to obtain B1-1.

B1-1 was added to dichloromethane (2 mL), then trifluoroacetic acid (1 mL) was added therein, and the mixture was stirred at room temperature for 30 minutes. The reaction solution was concentrated, and separated by silica gel column chromatography (dichloromethane:methanol=20:1) to obtain B6 as a pale yellow solid (245 mg, yield 90%). 1 H NMR (500 MHz, DMSO-d₆) δ 11.66 (s, 1H), 8.44 (s, 2H), 7.80 (d, J=8.0 Hz, 1H), 6.38 (s, 1H), 5.81 (d, J=7.2 Hz, 1H), 5.74-5.66 (m, 2H), 4.72 (dd, J=15.7, 12.3 Hz, 1H), 4.48 (dd, J=16.2, 12.4 Hz, 1H), 4.02 (d, J=4.4 Hz, 1H), 2.22-2.15 (m, 1H), 0.98 (d, J=6.9 Hz, 3H), 0.94 (d, J=6.9 Hz, 3H).

### Preparation Example 23: Synthesis of B13

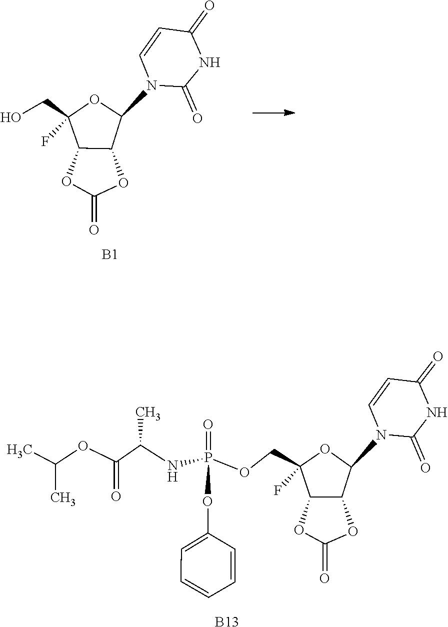

B1 (50.0 mg, 0.17 mmol), N-[(S)-(2,3,4,5,6-pentafluorophenoxy)phenoxyphosphoryl]-L-alanine isopropyl ester (86.7 mg, 0.19 mmol), anhydrous magnesium chloride (24.8 mg, 0.26 mmol) were added to anhydrous tetrahydrofuran (4 mL), then N,N-diisopropylethylamine (44.9 mg, 0.35 mmol) was added therein under an ice bath, the mixture was stirred for 10 minutes and then warmed to room temperature. After 4 hours, the reaction solution was concentrated; and water and ethyl acetate were added, and the organic phase was separated, sequencely washed with dilute hydrochloric acid, saturated sodium bicarbonate, and saturated brine, dried over anhydrous sodium sulfate, and separated by silica gel column chromatography (dichloromethane:methanol=30:1) to obtain B13 as a white solid (56 mg, yield 58%). ¹H NMR (500 MHz, CD₃OD) δ 7.67 (d, J=8.0 Hz, 1H), 7.37 (t, J=7.8 Hz, 2H), 7.26 (d, J=8.1 Hz, 2H), 7.20 (t, J=7.5 Hz, 1H), 6.15 (s, 1H), 5.70 (d, J=8.0 Hz, 1H), 5.68-5.64 (m, 2H), 5.02-4.94 (m, 1H), 4.43-4.36 (m, 1H), 4.35-4.27 (m, 1H), 3.96-3.89 (m, 1H), 1.36 (d, J=7.1 Hz, 3H), 1.25-1.21 (m, 6H).

### Preparation Example 24: Synthesis of B14

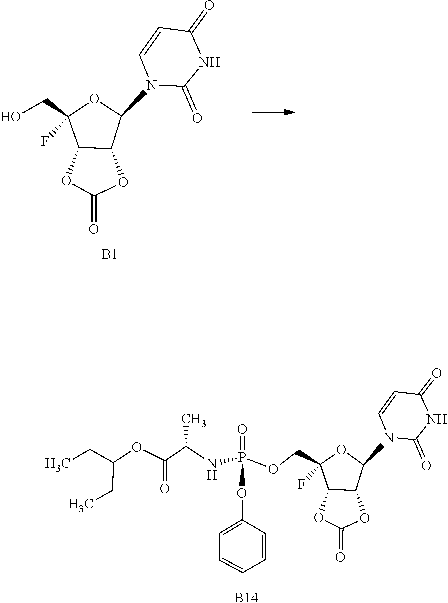

B1 (50.0 mg, 0.17 mmol), N-[(S)-(2,3,4,5,6-pentafluorophenoxy)phenoxyphosphoryl]-L-alanine ethyl-n-butyl ester (95.1 mg, 0.19 mmol), anhydrous magnesium chloride (24.8 mg, 0.26 mmol) were added to anhydrous tetrahydrofuran (3 mL), then N,N-diisopropylethylamine (44.9 mg, 0.35 mmol) was added under an ice bath, the mixture was stirred for 10 minutes and then warmed to room temperature. After 12 hours, the reaction solution was concentrated; and water and ethyl acetate were added, and the organic phase was separated, sequencely washed with dilute hydrochloric acid, saturated sodium bicarbonate, and saturated brine, dried over anhydrous sodium sulfate, and separated by silica gel column chromatography (dichloromethane:methanol=30:1) to obtain B14 as a white solid (51 mg, yield 50%). ¹H NMR (500 MHz, CD₃OD) δ 7.67 (d, J=7.9 Hz, 1H), 7.37 (t, J=7.8 Hz, 2H), 7.26 (d, J=8.1 Hz, 2H), 7.20 (t, J=7.5 Hz, 1H), 6.15 (s, 1H), 5.70 (d, J=8.0 Hz, 1H), 5.68-5.63 (m, 2H), 4.42-4.27 (m, 2H), 4.10-4.02 (m, 2H), 4.01-3.95 (m, 1H), 1.55-1.49 (m, 1H), 1.40-1.34 (m, 4H), 1.22 (d, J=6.2 Hz, 3H), 0.90 (t, J=7.4 Hz, 6H).

### Preparation Example 25: Synthesis of B44

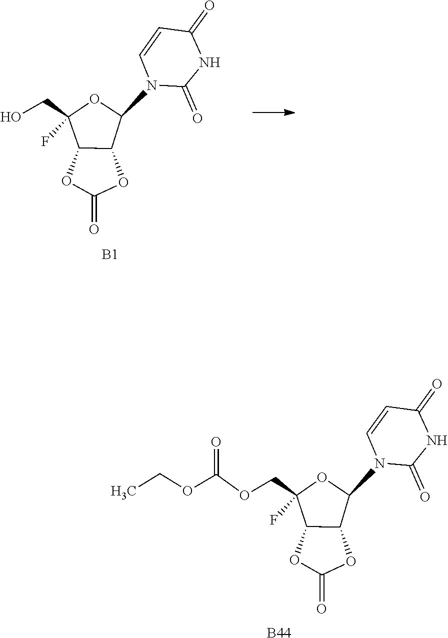

B1 (30.0 mg, 0.10 mmol), 4-dimethylaminopyridine (15.3 mg, 0.125 mmol), pyridine (0.1 mL) were added to dichloromethane (2 mL), and ethyl chloroformate (13.6 mg, 0.125 mmol) was added under an ice bath. After the addition was completed, the reaction solution was warmed to room temperature and stirred. After 4 hours, the reaction solution was concentrated; water and ethyl acetate were added, and the organic phase was separated, sequencely washed with dilute hydrochloric acid, saturated sodium bicarbonate, and saturated brine, dried over anhydrous sodium sulfate, and separated by silica gel column chromatography (dichloromethane:methanol=20:1) to obtain B44 as a white solid (32 mg, yield 89%). ¹H NMR (500 MHz, DMSO-d₆) δ 11.64 (s, 1H), 7.79 (d, J=8.0 Hz, 1H), 6.35 (s, 1H), 5.78 (d, J=8.0 Hz, 1H), 5.71 (dd, J=8.0, 2.2 Hz, 1H), 5.67 (dd, J=11.4, 7.3 Hz, 1H), 4.49-4.39 (m, 2H), 4.16 (q, J=7.1 Hz, 2H), 1.23 (t, J=7.1 Hz, 3H).

### Preparation Example 26: Synthesis of B38

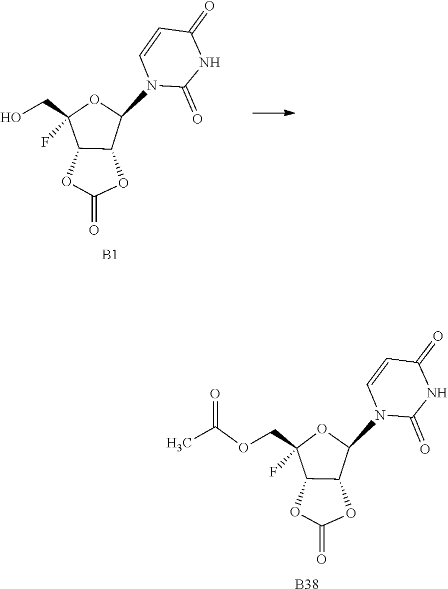

B1 (73.0 mg, 0.25 mmol) was added into dichloromethane (3 mL), and triethylamine (38.4 mg, 25 0.38 mmol), 4-dimethylaminopyridine (6.1 mg, 0.05 mmol), acetic anhydride (31.0 mg, 0.30 mmol) were sequencely added under an ice bath. After the addition, the reaction solution was warmed to room temperature and stirred. After 1 hour, dichloromethane (20 mL) was added to the reaction solution, and washed with 1 M hydrochloric acid aqueous solution, saturated sodium bicarbonate solution, and saturated brine successively. The organic phase was separated, dried over anhydrous sodium sulfate, concentrated, and separated by silica gel column chromatography (petroleum ether : ethyl acetate=1:1) to obtain B38 as a white solid (63 mg, yield 76%). ¹H NMR (500 MHz, DMSO-d₆) δ 11.63 (s, 1H), 7.79 (d, J=8.0 Hz, 1H), 6.34 (s, 1H), 5.77 (d, J=7.3 Hz, 1H), 5.71 (d, J=8.0 Hz, 1H), 5.64 (dd, J=11.7, 7.3 Hz, 1H), 4.46-4.31 (m, 2H), 2.08 (s, 3H).

### Preparation Example 27: Synthesis of C2

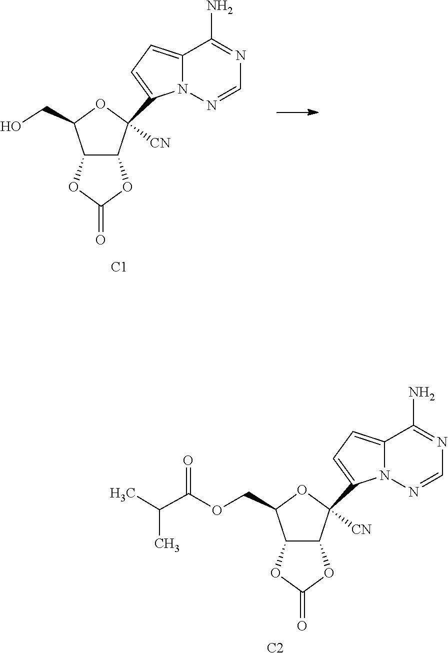

Referring to the reaction conditions of Preparation Example 11, C1 (95 mg, 0.3 mmol) was reacted with isobutyryl chloride (38 mg, 0.36 mmol) to obtain C2 as a white solid (27 mg, yield 23%). ¹H NMR (400 MHz, DMSO-d₆) δ 8.10 (s, 1H), 8.02 (s, 1H), 7.99 (s, 1H), 6.96 (d, J=4.6 Hz, 1H), 6.91 (d, J=4.6 Hz, 1H), 6.00 (d, J=7.7 Hz, 1H), 5.50 (dd, J=7.7, 3.7 Hz, 1H), 4.86-4.79 (m, 1H), 4.34 (dd, J=12.3, 4.0 Hz, 1H), 4.24 (dd, J=12.2, 5.2 Hz, 1H), 2.49-2.39 (m, 1H), 1.03 (d, J =7.0 Hz, 3H), 0.99 (d, J=7.0 Hz, 3H).

### Preparation Example 28: Synthesis of C23

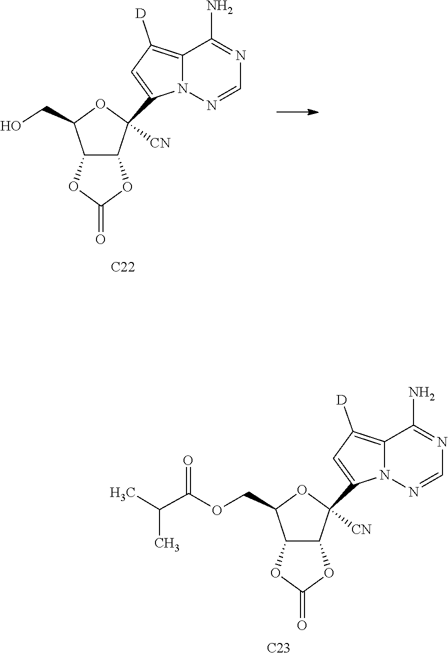 [SMILES: [2H]c1cc([C@]2(C#N)O[C@H](CO)[C@H]3OC(=O)O[C@H]32)n2ncnc(N)c12 ; "C22" ; [2H]c1cc([C@]2(C#N)O[C@H](COC(=O)C(C)C)[C@H]3OC(=O)O[C@H]32)n2ncnc(N)c12 ; "C23"]

Referring to the reaction conditions of Preparation Example 11, C22 (477 mg, 1.5 mmol) was reacted with isobutyryl chloride (192 mg, 0.18 mmol) to obtain C23 as a white solid (105 mg, yield 18%). 1 H NMR (500 MHz, DMSO-d₆) δ 8.11 (s, 1H), 8.03 (s, 1H), 7.98 (s, 1H), 6.90 (s, 1H), 5.99 (d, J=7.7 Hz, 1H), 5.49 (dd, J=7.6, 3.7 Hz, 1H), 4.82 (dt, J=5.2, 3.8 Hz, 1H), 4.33 (dd, J=12.3, 3.9 Hz, 1H), 4.22 (dd, J=12.3, 5.2 Hz, 1H), 2.47 -2.39 (m, 1H), 1.01 (d, J=7.0 Hz, 3H), 0.98 (d, J=7.0 Hz, 3H). 13 C NMR (126 MHz, DMSO-d₆) δ 175.5, 155.5, 152.9, 148.5, 120.0, 117.1, 114.1, 110.5, 82.7, 81.5, 80.1, 79.5, 62.1, 33.0, 18.6, 18.5.

### Preparation Example 29: Synthesis of C54

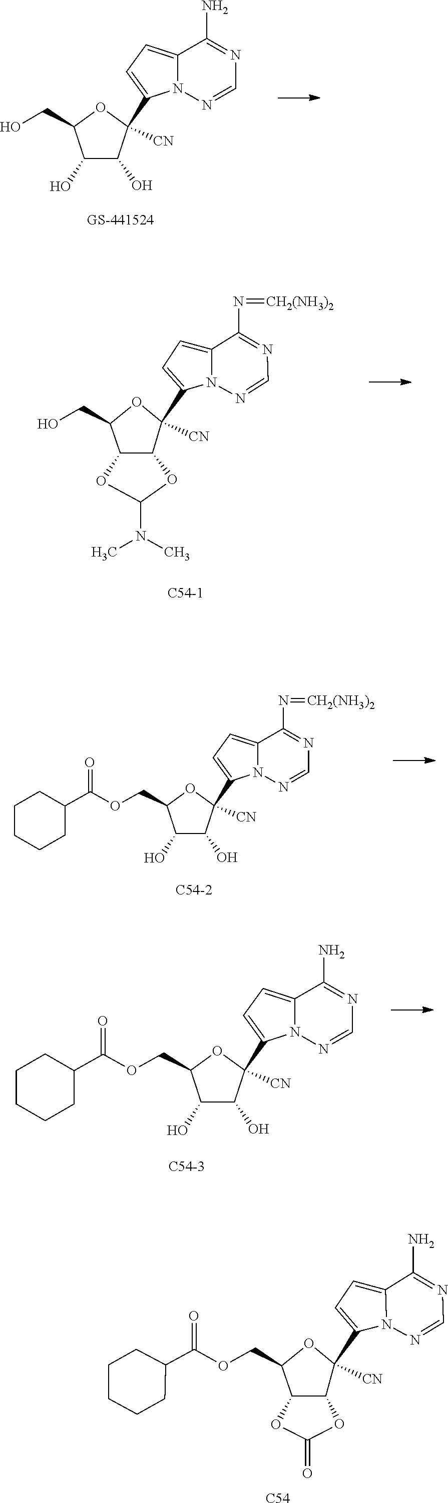

GS-441524 (611 mg, 2.1 mmol) was added to pyridine (5 mL), then N,N-dimethylformamide dimethyl acetal (1 g, 8.4 mmol) was added therein, and the mixture was stirred overnight at room temperature. The reaction solution was concentrated to dryness to obtain the crude C54-1, which was directly used for the next step.

C54-1 was added to dichloromethane (5 mL), and triethylamine (142 mg, 1.4 mmol), cyclobutylformyl chloride (125 mg, 1.05 mmol) and 4-dimethylaminopyridine (86 mg, 0.7 mmol) were sequencely added under an ice bath, and stirred at room temperature. After 1 hour, methanol was added to the reaction solution, the reaction solution was concentrated, ethyl acetate and water were added, the mixture was stirred and layered. The organic phase was separated, washed with dilute hydrochloric acid aqueous solution, saturated sodium bicarbonate and saturated sodium chloride successively, dried over anhydrous sodium sulfate, and evaporated to dryness to obtain C54-2.

C54-2 (0.35 mmol) was added to ethanol (3 mL), then acetic acid (0.6 mL, 10.5 mmol) was added therein, the mixture was heated and stirred at 50° C. overnight. The reaction solution was concentrated, saturated brine was added, and the mixture was extracted with ethyl acetate. The organic phase was separated, washed with saturated sodium bicarbonate and saturated brine, dried over anhydrous sodium sulfate, concentrated, and slurried in isopropyl acetate to obtain C54-3 as a white powdery solid (93 mg, yield 66.0% over three steps). ¹H NMR (500 MHz, DMSO-d₆) δ 8.06-7.79 (m, 3H), 6.93 (d, J=4.5 Hz, 1H), 6.82 (d, J=4.5 Hz, 1H), 6.34 (d, J=6.0 Hz, 1H), 5.38 (d, J=5.9 Hz, 1H), 4.74-4.68 (m, 1H), 4.31 (dd, J=12.2, 2.9 Hz, 1H), 4.26-4.21 (m, 1H), 4.15 (dd, J=12.2, 5.0 Hz, 1H), 4.00-3.94 (m, 1H), 2.30-2.22 (m, 1H), 1.81-1.54 (m, 5H), 1.34-1.11 (m, 5H).

C54-3 (100 mg, 0.25 mmol, 1 eq) was added to tetrahydrofuran (4 mL) under an ice bath, and carbonyldiimidazole (83 mg, 0.51 mmol, 2 eq) was added thereto. The ice bath was removed, and the reaction solution was stirred at room temperature for 3 h. Methanol was added, then 1 N dilute hydrochloric acid solution was added therein, and the mixture was extracted with ethyl acetate. The organic phase was washed with saturated aqueous sodium chloride solution, dried over anhydrous sodium sulfate, evaporated to dryness, and separated by column chromatography to obtain C54 as a white solid (72 mg, yield 67%). 1 H NMR (500 MHz, DMSO-d₆) δ 8.10 (s, 1H), 8.02 (s, 1H), 7.98 (s, 1H), 6.95 (d, J=4.6 Hz, 1H), 6.89 (d, J=4.6 Hz, 1H), 5.99 (d, J=7.5 Hz, 1H), 5.49 (dd, J=7.5, 3.4 Hz, 1H), 4.85 (q, J=3.8 Hz, 1H), 4.31 (dd, J=12.3, 3.7 Hz, 1H), 4.21 (dd, J=12.3, 5.0 Hz, 1H), 1.71-1.50 (m, 5H), 1.28-1.02 (m, 5H). MS m/z=428.4 [M+1]⁺.

### Preparation Example 30: Synthesis of B50

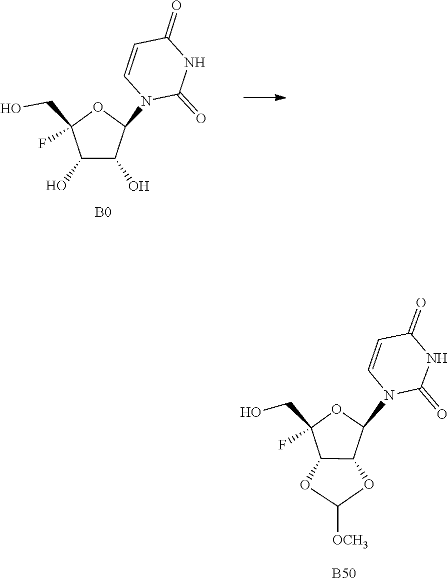

B0 was synthesized by the method in reference (WO2014100505). B0 (700.0 mg, 2.67 mmol, 1 eq) was added to trimethyl orthoformate (7 mL), then p-toluenesulfonic acid monohydrate (507.4 mg, 2.67 mmol, 1 eq) was added therein, and the mixture was reacted at room temperature. The reaction liquid gradually becomes clear. After TLC showed that the reaction was complete, the reaction solution was adjusted pH to 6-7 with a 7 N ammonia methanol solution. The reaction was filtered, and the filtrate was concentrated and separated by silica gel column chromatography (dichloromethane:methanol=60:1 to 40:1) to obtain B50 as a white foamy solid (yield 84%). ¹H NMR (500 MHz, DMSO-d₆) δ 11.52 (d, J=2.2 Hz, 1H), 7.73 (d, J=8.1 Hz, 1H), 6.09-6.05 (m, 2H), 5.67 (dd, J=8.0, 2.2 Hz, 1H), 5.47 (t, J=6.4 Hz, 1H), 5.29 (dd, J=14.3, 6.4 Hz, 1H), 5.11 (dd, J=6.4, 1.3 Hz, 1H), 3.65-3.53 (m, 2H), 3.22 (s, 3H).

### Test Example 1: In Vivo Pharmacokinetic Evaluation in Rats

### Experimental Method:

6 Male SD rats for each compound were divided into 2 groups (gastric administration group and intravenous injection group), 3 rats in each group. The rats were fasted for 12 h before the experiment (the intravenous experiment group was not fasted), had free access to water, and ate uniformly 4 hours after the administration. The dosage of A1 and A2 was 20 mg/kg for intragastric administration and 5 mg/kg for intravenous injection, and the administration vehicle was 5% DMSO +5% solutol+90% saline. The dosage of C38 was 10 mg/kg for intragastric administration, 2 mg/kg for intravenous injection, and the administration vehicle was DMSO/EtOH/PEG300/0.9% NaCl (5/5/40/50, v/v/v/v). 0.2 mL of blood was collected from the jugular vein at 5 min (intravenous group only), 0.25 h, 0.5 h, 1.0 h, 2.0 h, 4.0 h, 6.0 h, 8.0 h and 24 h after the administration, placed in an EDTA-K2 test tube and centrifuged at 11,000 rpm for 5 minutes, and the plasma was separated, and stored in a −70° C. refrigerator for testing. The operation was under an ice water bath. The concentration of nucleoside metabolites in plasma was determined by LC-MS-MS, and the pharmacokinetic parameters were calculated.

TABLE 1
In vivo pharmacokinetic parameters of nucleoside metabolites of A1, A2 and C38 in rats

| | Compound | | | | | |
|---|---|---|---|---|---|---|
| | A1 | A1 | A2 | A2 | C38 | C38 |
| PK parameters | (po-20mpk) | (iv-5mpk) | (po-20mpk) | (iv-5mpk) | (po-10mpk) | (iv-2mpk) |
| Tₘₐₓ(h) | 1.0 | — | 0.25 | — | 0.75 | — |
| Cₘₐₓ(ng/ml) | 678 | 531 | 937 | 871 | 586 | 217 |
| T_{1/2}(h) | 11.4 | 8.4 | 13.5 | — | 3.1 | 1.9 |
| AUC(0-t) | 2192 | 448 | 1559 | 418 | 2388 | 551 |
| (h*ng/mL) | | | | | | |
| AUC(0-∞) | 2374 | 495 | 1704 | 424 | 2454 | 586 |
| (h*ng/mL) | | | | | | |
| F % | 122% | | 93% | | 87% | |

mpk: mg/kg body weight

The PK test in rats showed that when A1, A2 and C38 were administered orally, the exposure of nucleoside metabolites was higher, and the bioavailability could reach 122.3%, 93% and 87% respectively.

### Test Example 2: Inhibition of Compounds on Viral Replication

Determination of the inhibitory activity of compounds on the replication of 2019 novel coronavirus (SARS-CoV-2): anti-novel coronavirus activity test methods of NHC and GS-441524 are as reported in the literature. To Vero cells infected with the 2019 novel coronavirus, different concentrations of test compounds were added. After 48 hours of incubation, the virus copy number in the cell supernatant was quantified by quantitative real-time RT-PCR (qRT-PCR) to evaluate the inhibitory activity of the compound on the virus (Sci Transl Med, 2020, 12:eabb5883; Cell Rep, 2020, 32:107940; Chinese Patent application No. 202010313870.X).

Determination of the inhibitory activity of compounds on the replication of respiratory syncytial virus (RSV), human coronavirus OC43, influenza A virus, and Zika virus by Cytopathogenic effect (CPE): the experimental cells were inoculated at a certain cell density to 96 well cell culture plates, and cultured overnight in a 5% CO₂, 37° C. incubator. The compounds and viruses were added the next day. Depending on the tested virus, the cells were cultured in an incubator for 3-7 days under 5% CO₂, 33° C. or 37° C., until 80-95% of the cells in the virus-infected control wells without compound had pathological changes. Cell viability in each well was then detected with CellTiter-Glo or CCK-8. If the cell viability of the compound-containing well is higher than that of the virus-infected control well, that is, the CPE is weakened, it indicates that the compound has an inhibitory effect on the tested virus. The cytotoxicity test method is the same as the corresponding antiviral test method, but without viral infection.

The antiviral activity and cytotoxicity of the compound are represented by the inhibition rate (%) of the compound on the virus-induced cellular viral effect and the cell viability (%), respectively. Calculation equations are as follows:

Inhibition rate (%)=(reading value of test well−average value of virus control)/(average value of cell control−average value of virus control)×100;

Cell viability (%)=(reading value of test well−average value of medium control)/(average value of cell control−average value of medium control)×100;

EC50 and CC50 values were calculated by Prism software, and the inhibition curve fitting method was “log (inhibitor) vs. response—Variable slope”.

Determination of the inhibitory activity of compounds against dengue virus by plaque reduction assay: Vero cells were inoculated into 6-well cell culture plates at a density of 600,000 cells per well, and cultured overnight in a 5% CO₂, 37° C. incubator. The compounds and virus (40-50 PFU/well) were added the next day. The cells were cultured in an incubator at 5% CO₂ and 37° C. for 2 hours, then the supernatant was aspirated, and the low-melting point agarose culture medium containing the corresponding concentration of the compound was added. The cells were cultured in an incubator at 5% CO₂, 33° C. or 37° C. for 6-7 days, until obvious virus plaques could be observed in the virus-infected control wells without compound under the microscope. Cells were fixed with 4% paraformaldehyde and stained with crystal violet. The number of plaques in each well was calculated. Cytotoxicity experiments were performed in parallel with antiviral experiments. Vero cells were inoculated into 96-well cell culture plates at a density of 20,000 cells per well, and cultured overnight in a 5% CO₂, 37° C. incubator. The compounds were added the next day (1-5 concentration points, single point). The cells were cultured in an incubator at 5% CO₂, 33° C. or 37° C. for 6-7 days. Cell viability in each well was then detected with CCK-8.

Determination of inhibitory activity of the compound on the replication of porcine epidemic diarrhea virus (PEDV) by Fluorescent quantitative PCR: Vero cells were digested and passaged, adjusted the cell density to 1×10⁵/mL with cell growth medium, inoculated in a 96 well plate with 100 μL/well, and placed in a 37° C., 5% CO₂ incubator to incubate for 24 hours. The 96 well plate was taken out with the medium in the wells be discarded, washed three times with 1×PBS, spin-dried, added with a mixed solution of the compound (10 concentration points) and the virus (0.01 MOI per well) in each well, with 8 replicate wells for each concentration, and incubated in a 37° C., 5% CO₂ incubator, setting virus control and cell control at the same time. After 36 hours, the cell samples were collected, and the changes of virus content in different treatment groups were measured by fluorescent quantitative PCR, and the EC₅₀ of the compound was calculated.

TABLE 2
Inhibitory activity against novel coronavirus (SARS-COV-2) and human coronavirus
OC43 (HCoV OC43)

| | | SARS-COV-2 (Vero) | | HCoV OC43 (Huh7) | |
|---|---|---|---|---|---|
| Compound | Structure (free state) | EC₅₀ (μM) | CC₅₀ (μM) | EC₅₀ (μM) | CC₅₀ (μM) |
| NHC | 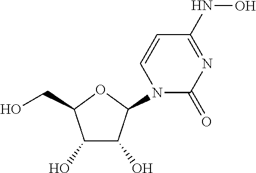 [SMILES: O=c1nc(NO)ccn1[C@@H]1O[C@H](CO)[C@@H](O)[C@H]1O] | 0.30 | >10 | — | — |
| C22-0 | 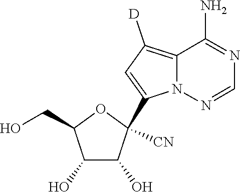 [SMILES: [2H]c1cc([C@]2(C#N)O[C@H](CO)[C@@H](O)[C@H]2O)n2ncnc(N)c12] | 0.46 | >10 | 1.77 | >10 |
| GS-441524 | 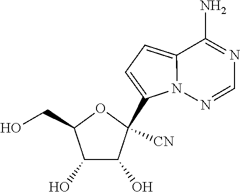 [SMILES: N#C[C@@]1(c2ccc3c(N)ncnn23)O[C@H](CO)[C@@H](O)[C@H]1O] | 0.47 | >10 | 1.88 | >10 |

Table 3: Inhibitory Activity Against Respiratory Syncytial Virus (RSV) and Influenza Virus

TABLE 3
inhibitory activity against respiratory syncytial virus (RSV) and influenza virus

| | | RSV (HEp-2) | | H3N2 (MDCK) | |
|---|---|---|---|---|---|
| Compound | Structure (free state) | EC₅₀ (μM) | CC₅₀ (μM) | EC₅₀ (μM) | CC₅₀ (μM) |
| NHC | 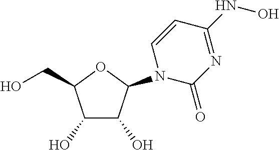 [SMILES: O=c1nc(NO)ccn1[C@@H]1O[C@H](CO)[C@@H](O)[C@H]1O] | 4.6 | >10 | 0.77 | >10 |
| A1 | 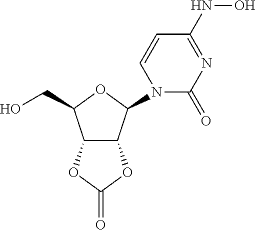 [SMILES: O=C1O[C@@H]2[C@H](O1)[C@@H](CO)O[C@H]2n1ccc(NO)nc1=O] | | — | 2.6 | — |
| A2 | 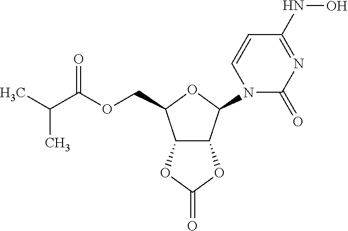 [SMILES: CC(C)C(=O)OC[C@H]1O[C@@H](n2ccc(NO)nc2=O)[C@@H]2OC(=O)O[C@@H]21] | — | — | >10 | >10 |
| B1-N | 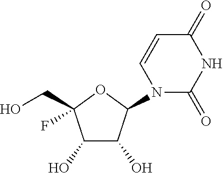 [SMILES: O=c1ccn([C@@H]2O[C@](F)(CO)[C@@H](O)[C@H]2O)c(=O)[nH]1] | — | — | <1.0 | >10 |
| B1 | 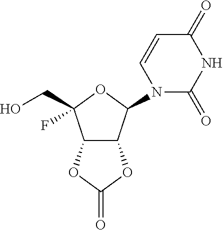 [SMILES: O=C1O[C@H]2[C@H](n3ccc(=O)[nH]c3=O)O[C@](F)(CO)[C@H]2O1] | 0.99 | >10 | 0.37 | >10 |
| B2 | 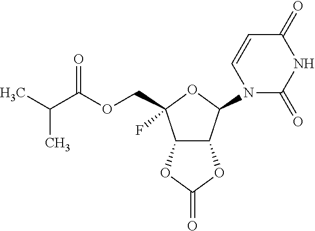 [SMILES: CC(C)C(=O)OC[C@@]1(F)O[C@@H](n2ccc(=O)[nH]c2=O)[C@@H]2OC(=O)O[C@@H]21] | > 10 | >10 | >10 | >10 |
| B4 | 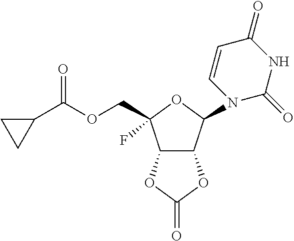 [SMILES: O=C1O[C@H]2[C@H](n3ccc(=O)[nH]c3=O)O[C@](F)(COC(=O)C3CC3)[C@H]2O1] | 1.02 | >10 | 1.38 | >10 |
| B6 | 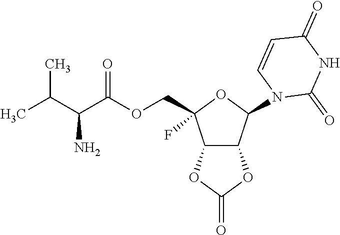 [SMILES: CC(C)[C@H](N)C(=O)OC[C@@]1(F)O[C@@H](n2ccc(=O)[nH]c2=O)[C@@H]2OC(=O)O[C@@H]21] | 3.46 | >10 | <0.5 | >10 |
| B38 | 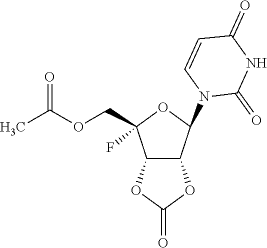 [SMILES: CC(=O)OC[C@@]1(F)O[C@@H](n2ccc(=O)[nH]c2=O)[C@@H]2OC(=O)O[C@@H]21] | 0.99 | >10 | 0.54 | >10 |
| B41 | 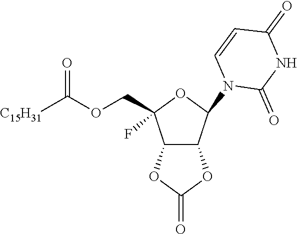 | 0.46 | >10 | <0.5 | >10 |
| B14 | 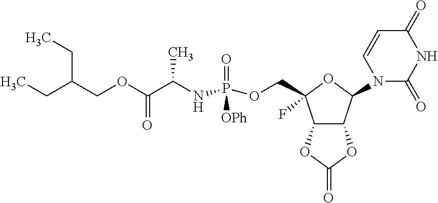 [SMILES: CCC(CC)COC(=O)[C@H](C)N[P@](=O)(OC[C@@]1(F)O[C@@H](n2ccc(=O)[nH]c2=O)[C@@H]2OC(=O)O[C@@H]21)Oc1ccccc1] | 1.08 | >10 | — | — |
| C22-0 | 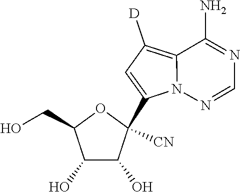 [SMILES: [2H]c1cc([C@]2(C#N)O[C@H](CO)[C@@H](O)[C@H]2O)n2ncnc(N)c12] | 0.56 | >10 | — | — |
| GS-441524 | 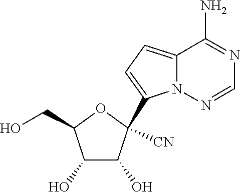 [SMILES: N#C[C@@]1(c2ccc3c(N)ncnn23)O[C@H](CO)[C@@H](O)[C@H]1O] | 0.73 | >10 | — | — |
| C21 | 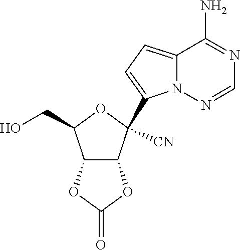 [SMILES: N#C[C@@]1(c2ccc3c(N)ncnn23)O[C@H](CO)[C@H]2OC(=O)O[C@H]21] | 0.49 | >10 | — | — |

TABLE 4
Inhibitory activity against replication of Porcine Epidemic Diarrhea Virus (PEDV),
Zika Virus (Zika) and Dengue Virus (DENV)

| | | | | DENV (Vero) |
|---|---|---|---|---|
| | | PEDV (Vero) | Zika (Huh7) | Inhibition rate |
| Compound | Structure | EC₅₀ (μM) | EC₅₀ (μM) | at 5 μM |
| NHC | 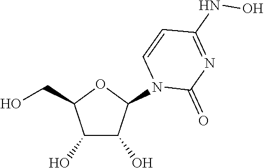 [SMILES: O=c1nc(NO)ccn1[C@@H]1O[C@H](CO)[C@@H](O)[C@H]1O] | 1.17 | Inhibition rate of 66% (5 μM) | 40% |
| B1-N | 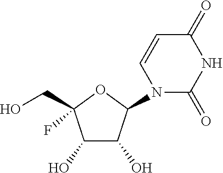 [SMILES: O=c1ccn([C@@H]2O[C@](F)(CO)[C@@H](O)[C@H]2O)c(=O)[nH]1] | — | Inhibition rate of 30% (5 μM) | — |
| GS-441524 | 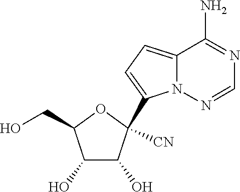 [SMILES: N#C[C@@]1(c2ccc3c(N)ncnn23)O[C@H](CO)[C@@H](O)[C@H]1O] | 0.31 | 15 | / |
| C22-0 | 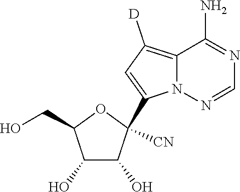 [SMILES: [2H]c1cc([C@]2(C#N)O[C@H](CO)[C@@H](O)[C@H]2O)n2ncnc(N)c12] | — | — | 100% |

### Test Example 3: In Vivo Pharmacokinetic Evaluation in Rats

A total of 18 male SD rats were divided into intravenous group (IV) and intragastric administration group (PO). They were fasted for 12 h before the experiment (the intravenous group was not fasted), had free access to water, and ate uniformly 4 h after administration. The dosages of B1, B2 and B6 were 76 μmol/kg (n=3) for intragastric administration and 38 μmol/kg (n=3) for intravenous injection, and the administration vehicle is DMSO/EtOH/PEG400/0.9% NaCl (5/5/40/50, v/v/v/v). 0.2-0.3 mL of blood was collected from the jugular vein 5 min (intravenous group only), 0.25 h, 0.5 h, 1.0 h, 2.0 h, 4.0 h, 6.0 h, 8.0 h and 24 h after administration, placed in a sodium heparin anticoagulant tube, mixed gently and centrifuged at 2000 g for 10 minutes, and the plasma was separated and stored in a −70° C. refrigerator for testing. The concentration of the nucleoside metabolite in plasma was determined by LC-MS-MS, and the pharmacokinetic parameters were calculated.

TABLE 5
In vivo pharmacokinetic parameters of nucleoside metabolites in
rats for single oral administration (76 μmol/kg) and injection
administration (38 μmol/kg) of B1, B2 and B6

| | Compound | | | | | |
|---|---|---|---|---|---|---|
| | B1 | B1 | B2 | B2 | B6 | B6 |
| PK parameters | (po) | (iv) | (po) | (iv) | (po) | (iv) |
| Tₘₐₓ(h) | 4.0 | — | 2.0 | — | 2.67 | — |
| Cₘₐₓ(ng/ml) | 1122 | 2590 | 3930 | 4543 | 2410 | 2933 |
| T_{1/2}(h) | 6.03 | 6.24 | 5.47 | 6.60 | 4.93 | 5.31 |
| AUC(0-t) | 11638 | 9820 | 29836 | 18081 | 18472 | 13874 |
| (h*ng/ml) | | | | | | |
| AUC(0-∞) | 12359 | 10385 | 31111 | 19265 | 19051 | 14345 |
| (h*ng/ml) | | | | | | |
| F % | 59% | | 83% | | 67% | |

### Test Example 4: In Vivo Pharmacokinetic Evaluation in Cynomolgus Monkeys

A total of 6 cynomolgus monkeys were given a single intragastric administration of A1, A2 and A6 (0.35 mmol/kg, n=2), and 10 blood samples were collected from each monkey within 48 hours after administration for LC-MS/MS analysis, to detect the concentrations of nucleoside metabolites NHC and Al and calculate conventional pharmacokinetic parameters, and the data were summarized.

TABLE 6
In vivo pharmacokinetic parameters of the nucleoside
metabolite in monkeys for single intragastric administration
(0.35 mmol/kg) of Molnupiravir (control, 5′-isobutyrate
prodrug of NHC), A1, A2 and A6

| | T_{1/2} | Tₘₐₓ | Cₘₐₓ | AUC₀₋ₜ |
|---|---|---|---|---|
| Compound | (h) | (h) | (ng/mL) | (ng · h/mL) |
| Molnupiravir | 0.50 | 1.50 | 2670 | 6337 |
| A1 | 1.26 | 1.50 | 195 | 591 |
| A2 | 0.63 | 1.00 | 3620 | 7435 |
| A6 | 0.83 | 1.50 | 1209 | 2417 |

TABLE 7
In vivo pharmacokinetic parameters of A1 in monkeys for single
intragastric administration (0.35 mmol/kg) of A1, A2 and A6

| | t_{1/2} | Tₘₐₓ | Cₘₐₓ | AUC₀₋ₜ |
|---|---|---|---|---|
| Compound | (h) | (h) | (ng/mL) | (ng · h/mL) |
| A1 | 1.20 | 1.50 | 90.8 | 365 |
| A2 | 0.88 | 1.00 | 684 | 1562 |
| A6 | 0.91 | 1.50 | 326 | 818 |

### Test Example 5

### In Vivo Pharmacokinetic Evaluation in Rats

### Experimental Method:

6 Male SD rats for each compound were divided into 2 groups (gastric administration group and intravenous injection group), 3 rats in each group. The rates were fasted for 12 h before the experiment (the intravenous experiment group was not fasted), had free access to water, and ate uniformly 4 hours after the administration. The dosages of C22 and C23 were 16 mg/kg and 20 mg/kg for intragastric administration, respectively, and the administration vehicle was 5% DMSO+5% solutol+90% saline. The intravenous injection dosages were 4 mg/kg and 5 mg/kg respectively, and the administration vehicle was DMSO/EtOH/PEG300/0.9% NaCl (5/5/40/50, v/v/v/v). 0.2 mL of blood was collected from the jugular vein 5 min, 0.25 h, 0.5 h, 1.0 h, 2.0 h, 4.0 h, 6.0 h, 8.0 h and 24 h after the administration, placed in an EDTA-K2 test tube abd centrifuged at 11,000 rpm for 5 minutes, and the plasma was separated, and stored in a −70° C. refrigerator for testing. The operation was under an ice water bath. The concentration of nucleoside metabolites in plasma was determined by LC-MS-MS, and the pharmacokinetic parameters were calculated.

TABLE 8
In vivo pharmacokinetic parameters of nucleoside
metabolites of C22 and C23 in rats

| | Compound | | | |
|---|---|---|---|---|
| | C22 | C22 | C23 | C23 |
| PK parameters | (po-16 mpk) | (iv-4 mpk) | (po-20 mpk) | (iv-5 mpk) |
| Tₘₐₓ(h) | 0.5 | — | 1.0 | — |
| Cₘₐₓ(ng/mL) | 2217 | 2883 | 2933 | 3903 |
| T_{1/2}(h) | 1.08 | 1.35 | 3.47 | 5.24 |
| AUC(0-t) | 7068 | 2348 | 11360 | 3954 |
| (h*ng/mL) | | | | |
| AUC(0-∞) | 7157 | 2398 | 11393 | 4024 |
| (h*ng/mL) | | | | |
| F % | 75.3% | | 71.8% | |

mpk: mg/kg body weight

The PK test in rats showed that when C22 and C23 were administered orally, the exposure of nucleoside metabolites was higher, and the bioavailability could reach 75.3% and 71.8% respectively.

According to the above test examples and the results of Tables 1-8, it can be seen that some compounds of the present application have high oral bioavailability and significant inhibitory activity against various viruses, and thus have good prospect in antiviral application.

1. A compound represented by formula I or a pharmaceutically acceptable salt thereof: 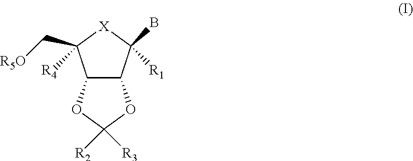
wherein
B is selected from the group consisting of 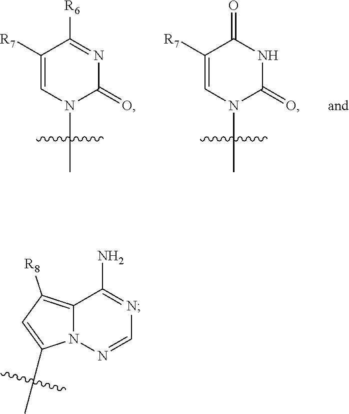
X is selected from the group consisting of oxygen, sulfur, CH₂, and NH;
R₁ is selected from the group consisting of hydrogen, deuterium, and cyano;
R₂ is selected from the group consisting of hydrogen, C₁₋₁₈ alkyl, C₃₋₈ cycloalkyl, C₈₋₂₀ aryl, and 5-15 membered heteroaryl, wherein the alkyl and the cycloalkyl are unsubstituted or substituted by one to three substituents independently selected from the group consisting of halogen, hydroxyl, carboxyl and 0 1-4 alkoxy, and the aryl and the heteroaryl are unsubstituted or substituted by one to five substituents independently selected from the group consisting of R₉;
R 3 is selected from the group consisting of hydrogen, and C₁₋₄ alkoxy; 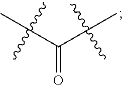 [SMILES: *C(*)=O]
or R₂, R₃ and the carbon to which they are attached form
R₄ is selected from the group consisting of hydrogen, deuterium, halogen, azido, cyano, C₁₋₈ alkyl, halogenated C₁₋₆ alkyl, azido C₁₋₈ alkyl, cyano C₁₋₆ alkyl, hydroxy C₁₋₆ alkyl, C₂₋₆ alkenyl, C₂₋₈ alkynyl, C₃₋₈ cycloalkyl, and C₁₋₈ alkoxy C₁₋₆ alkyl;
R₅ is selected from the group consisting of hydrogen, C₁₋₂₀ alkanoyl, C₃₋₂₀ cycloalkanoyl, amino C₁₋₂₀ alkanoyl, C₁₋₂₀ alkylamino C₁₋₈ alkanoyl, C₁₋₆ cycloalkylamino C₁₋₈ alkanoyl, C₁₋₂₀ dialkylamino C₁₋₈ alkanoyl, C₁₋₂₀ alkoxy C₁₋₈ alkanoyl, an amino acid group in which the carbonyl of the carboxyl group on the amino acid forms an ester bond with the connected oxygen, C₆₋₂₀ arylamino C₁₋₆ alkanoyl, 3-20 membered heterocycloalkyl C₁₋₆ alkanoyl, 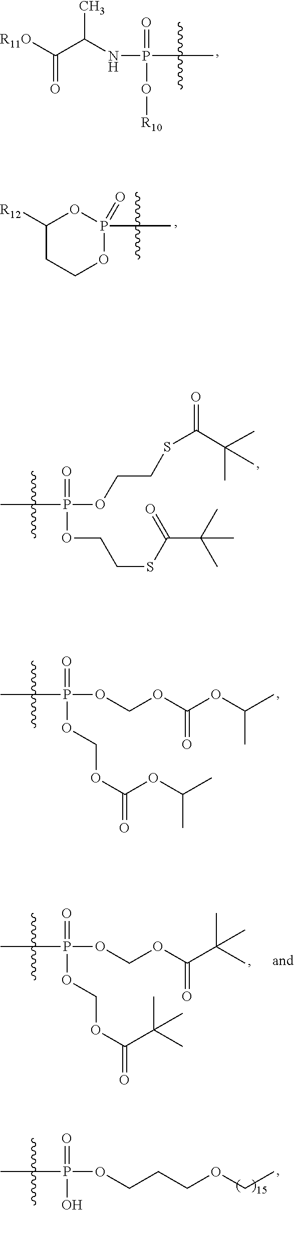
wherein the C₁₋₂₀ alkanoyl and the C₃₋₂₀ cycloalkanoyl are unsubstituted or substituted by one to three halogens, and the 3-20 membered heterocycloalkyl is unsubstituted or substituted by C₁₋₆ alkyl;
R₆ is selected from the group consisting of hydroxyl, amino, hydroxylamine (—NHOH), and —NHOR_{13;}
R₇ is selected from the group consisting of hydrogen, deuterium, and halogen;
R₈ is selected from the group consisting of hydrogen, deuterium, halogen, cyano, and carbamoyl;
R₉ is selected from the group consisting of halogen, C₁₋₄ alkyl, C₁₋₄ alkoxy, C₁₋₄ alkylthio, cyano, nitro, amino, phenyl, carboxyl, trifluoromethyl, difluoromethoxy, trifluoromethoxy, C₁₋₄ alkylamino, di(C₁₋₄ alkyl) amino, C₁₋₄ alkylcarbonyl, C₁₋₄ alkylcarbonyloxy, and C₁₋₄ alkoxycarbonyl;
R₁₀ is selected from the group consisting of C₁₋₆ alkyl, C₃₋₆ cycloalkyl, C₆₋₂₀ aryl, and 5-15 membered heteroaryl;
R₁₁ is selected from the group consisting of C₁₋₁₈ alkyl, and methylene C₆₋₂₀ aryl;
R₁₂ is selected from the group consisting of C₁₋₆ alkyl, C₃₋₆ cycloalkyl, C₆₋₂₀ aryl, and 5-15 membered heteroaryl;
R₁₃ is selected from the group consisting of C₁₋₂₀ alkanoyl, C₃₋₂₉ cycloalkanoyl, amino C₁₋₂₀ alkanoyl, C₁₋₂₉ alkylamino C₁₋₆ alkanoyl, C₁₋₆ cycloalkylamino C₁₋₆ alkanoyl, C₁₋₂₀ dialkylamino C₁₋₆ alkanoyl, C₁₋₂₀ alkoxy C₁₋₆ alkanoyl, and C₁₋₆ alkoxycarbonyloxymethylene.

2. The compound or the pharmaceutically acceptable salt thereof according to claim 1, wherein the compound is represented by formula I-I: 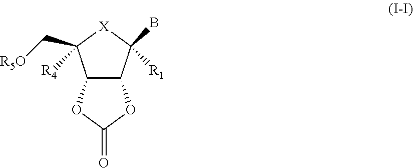
wherein, B, X, R₁, R₄, and R₅ are defined the same as those in the claim 1.

3. The compound or the pharmaceutically acceptable salt thereof according to claim 1, wherein the compound is represented by formula I-II: 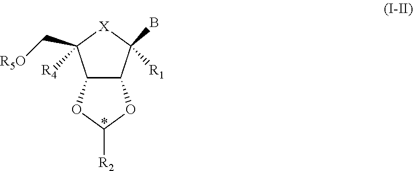
Preferably, the formula (I-II) is selected from the group consisting of formulas I-IIA and I-IIB, 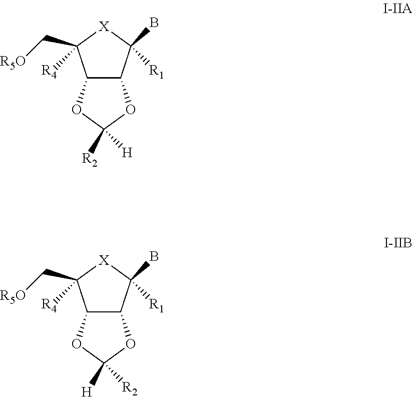
wherein, B, X, R₁, R₂, R₄ and R₅ are defined the same as those in the claim 1.

4. The compound or the pharmaceutically acceptable salt thereof according to claim 1, wherein the compound is selected from the group consisting of the following formulas: 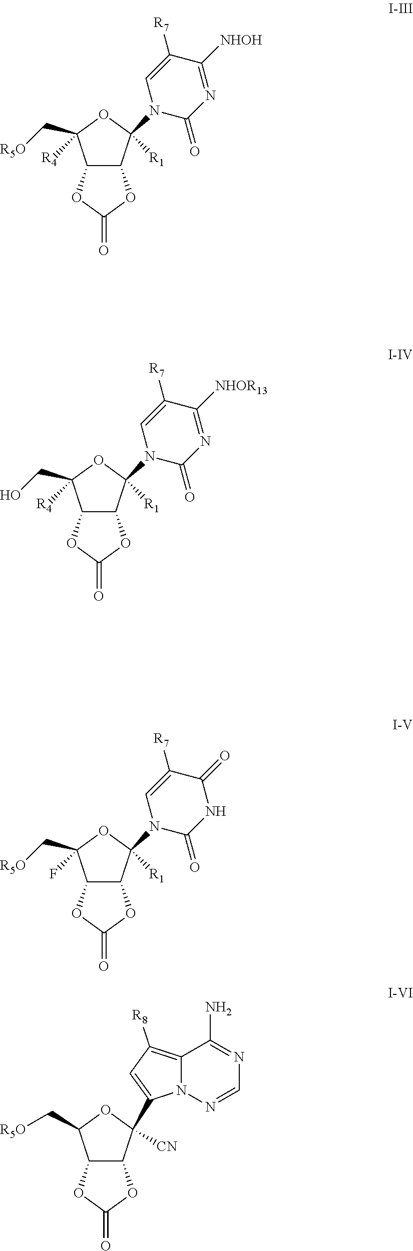 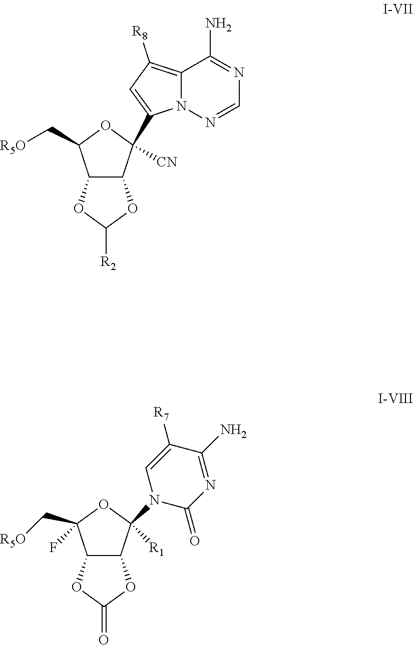
wherein
R₁ is selected from the group consisting of hydrogen and deuterium;
R₂, R₅, R₇, R₈ and R₁₃ are defined the same as those in the claim 1;
R₄ is selected from the group consisting of hydrogen, deuterium and halogen.

5. The compound or the pharmaceutically acceptable salt thereof according to claim 1, wherein the compound is selected from the group consisting of the following compounds:

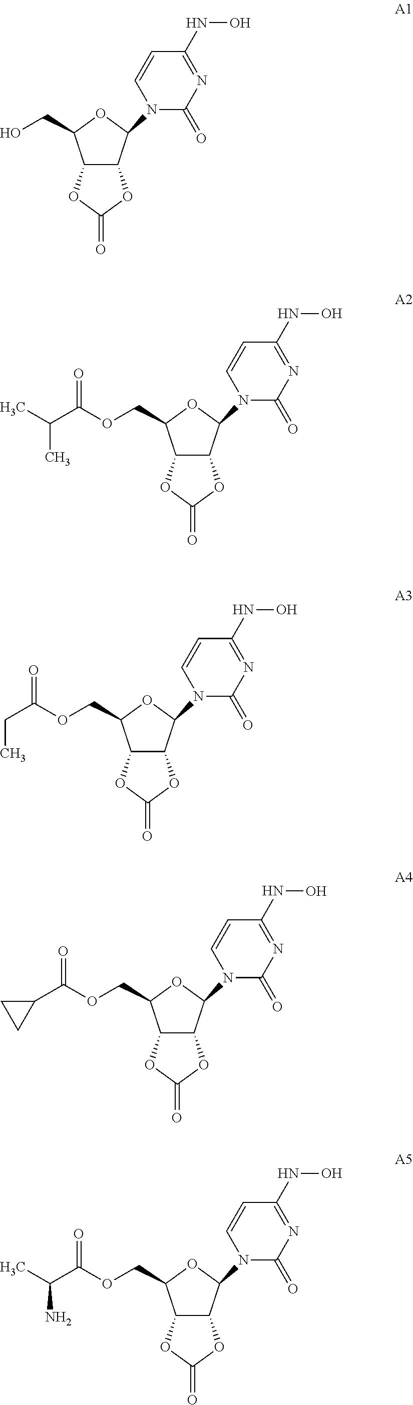 [SMILES: O=C1O[C@@H]2[C@H](O1)[C@@H](CO)O[C@H]2n1ccc(NO)nc1=O ; CC(C)C(=O)OC[C@H]1O[C@@H](n2ccc(NO)nc2=O)[C@@H]2OC(=O)O[C@@H]21 ; CCC(=O)OC[C@H]1O[C@@H](n2ccc(NO)nc2=O)[C@@H]2OC(=O)O[C@@H]21 ; O=C1O[C@@H]2[C@H](O1)[C@@H](COC(=O)C1CC1)O[C@H]2n1ccc(NO)nc1=O ; C[C@H](N)C(=O)OC[C@H]1O[C@@H](n2ccc(NO)nc2=O)[C@@H]2OC(=O)O[C@@H]21]

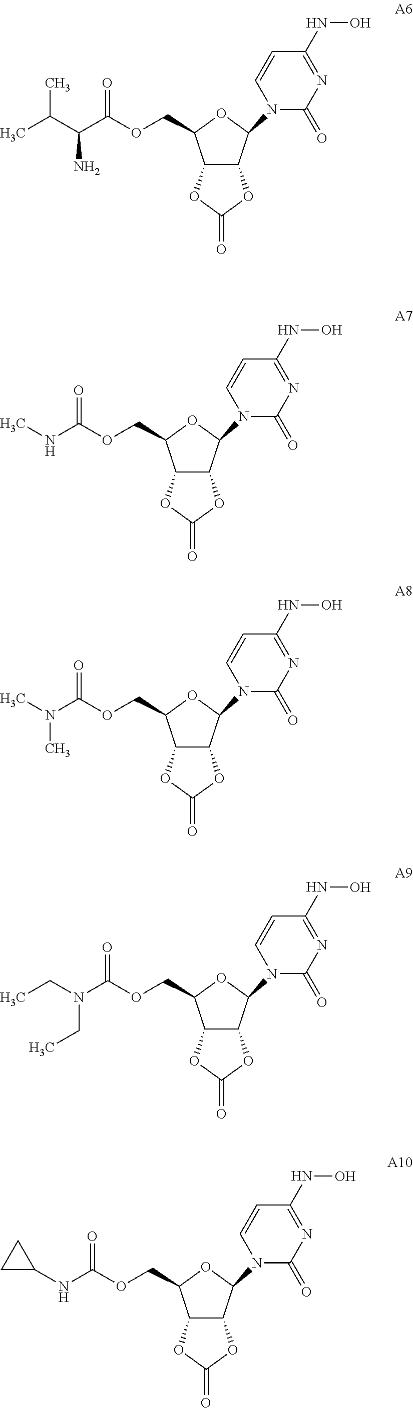 [SMILES: CC(C)[C@H](N)C(=O)OC[C@H]1O[C@@H](n2ccc(NO)nc2=O)[C@@H]2OC(=O)O[C@@H]21 ; CNC(=O)OC[C@H]1O[C@@H](n2ccc(NO)nc2=O)[C@@H]2OC(=O)O[C@@H]21 ; CN(C)C(=O)OC[C@H]1O[C@@H](n2ccc(NO)nc2=O)[C@@H]2OC(=O)O[C@@H]21 ; CCN(CC)C(=O)OC[C@H]1O[C@@H](n2ccc(NO)nc2=O)[C@@H]2OC(=O)O[C@@H]21 ; O=C(NC1CC1)OC[C@H]1O[C@@H](n2ccc(NO)nc2=O)[C@@H]2OC(=O)O[C@@H]21]

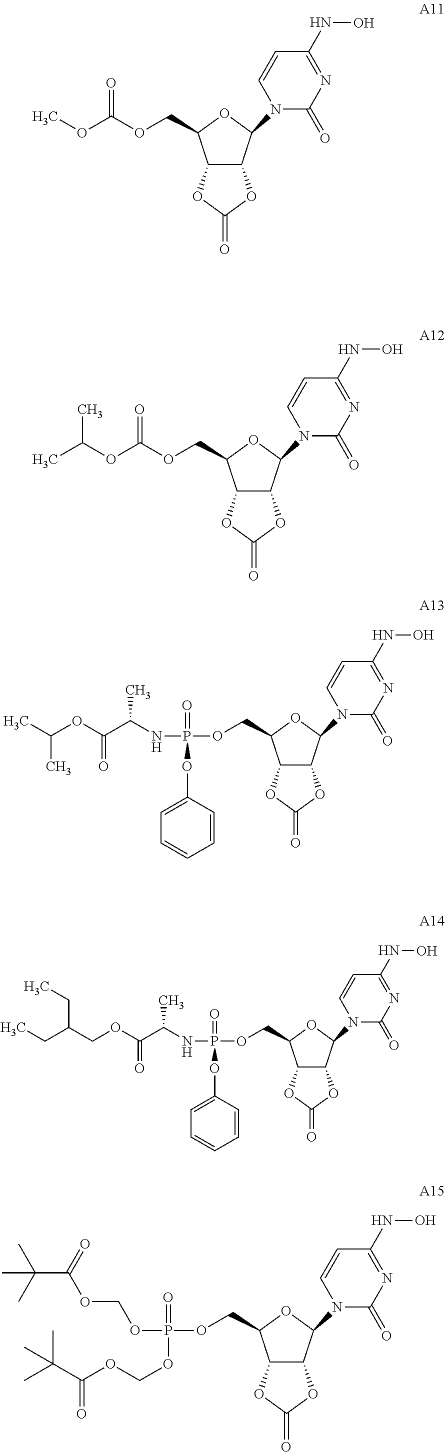 [SMILES: COC(=O)OC[C@H]1O[C@@H](n2ccc(NO)nc2=O)[C@@H]2OC(=O)O[C@@H]21 ; CC(C)OC(=O)OC[C@H]1O[C@@H](n2ccc(NO)nc2=O)[C@@H]2OC(=O)O[C@@H]21 ; CC(C)OC(=O)[C@H](C)N[P@](=O)(OC[C@H]1O[C@@H](n2ccc(NO)nc2=O)[C@@H]2OC(=O)O[C@@H]21)Oc1ccccc1 ; CCC(CC)COC(=O)[C@H](C)N[P@](=O)(OC[C@H]1O[C@@H](n2ccc(NO)nc2=O)[C@@H]2OC(=O)O[C@@H]21)Oc1ccccc1 ; CC(C)(C)C(=O)OCOP(=O)(OCOC(=O)C(C)(C)C)OC[C@H]1O[C@@H](n2ccc(NO)nc2=O)[C@@H]2OC(=O)O[C@@H]21]

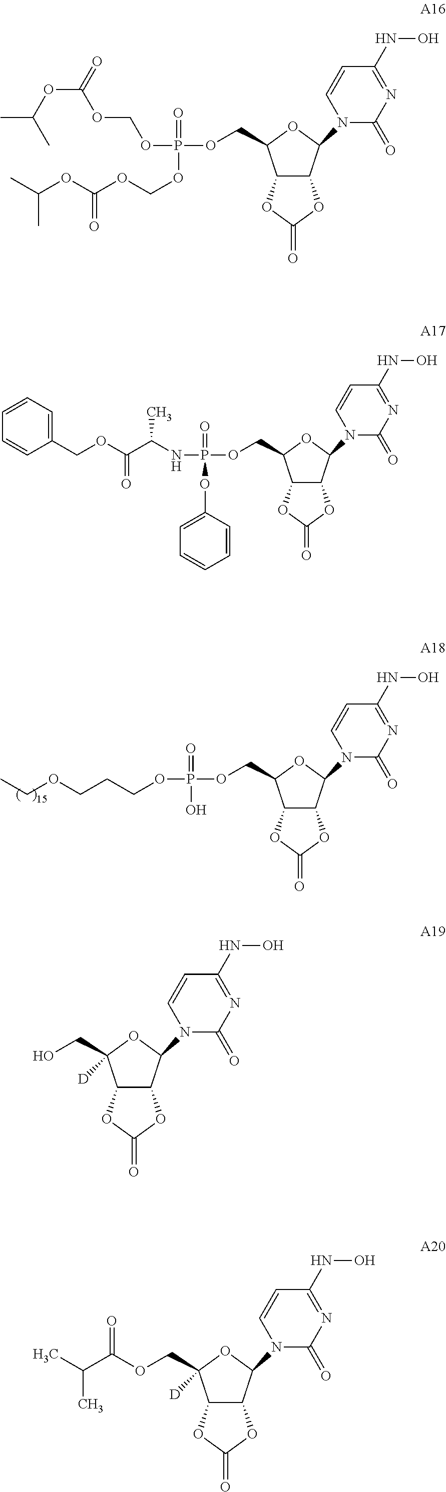

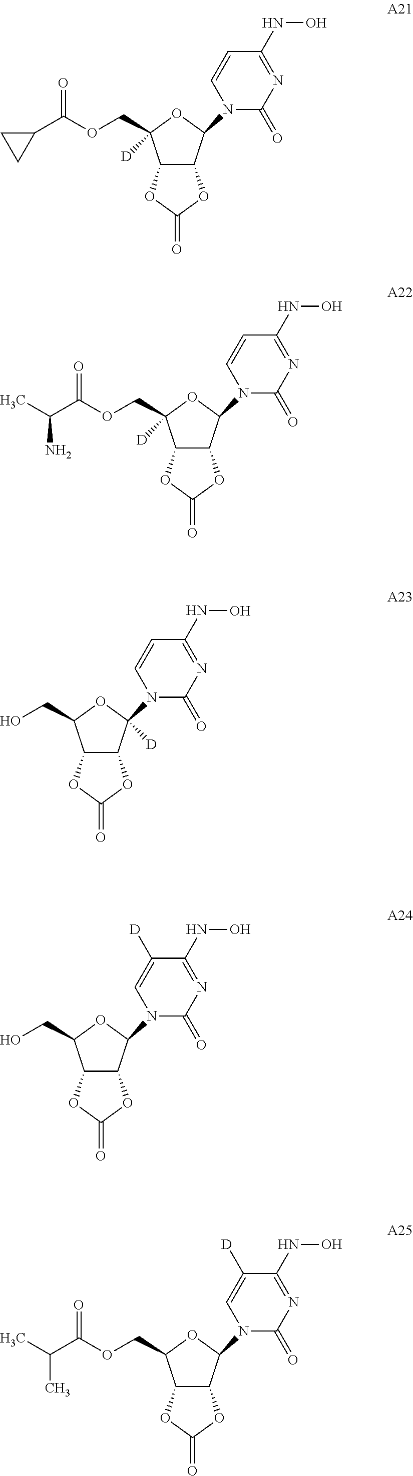 [SMILES: [2H][C@]1(COC(=O)C2CC2)O[C@@H](n2ccc(NO)nc2=O)[C@@H]2OC(=O)O[C@@H]21 ; [2H][C@]1(COC(=O)[C@H](C)N)O[C@@H](n2ccc(NO)nc2=O)[C@@H]2OC(=O)O[C@@H]21 ; [2H][C@@]1(n2ccc(NO)nc2=O)O[C@H](CO)[C@H]2OC(=O)O[C@H]21 ; [2H]c1cn([C@@H]2O[C@H](CO)[C@H]3OC(=O)O[C@H]32)c(=O)nc1NO ; [2H]c1cn([C@@H]2O[C@H](COC(=O)C(C)C)[C@H]3OC(=O)O[C@H]32)c(=O)nc1NO]

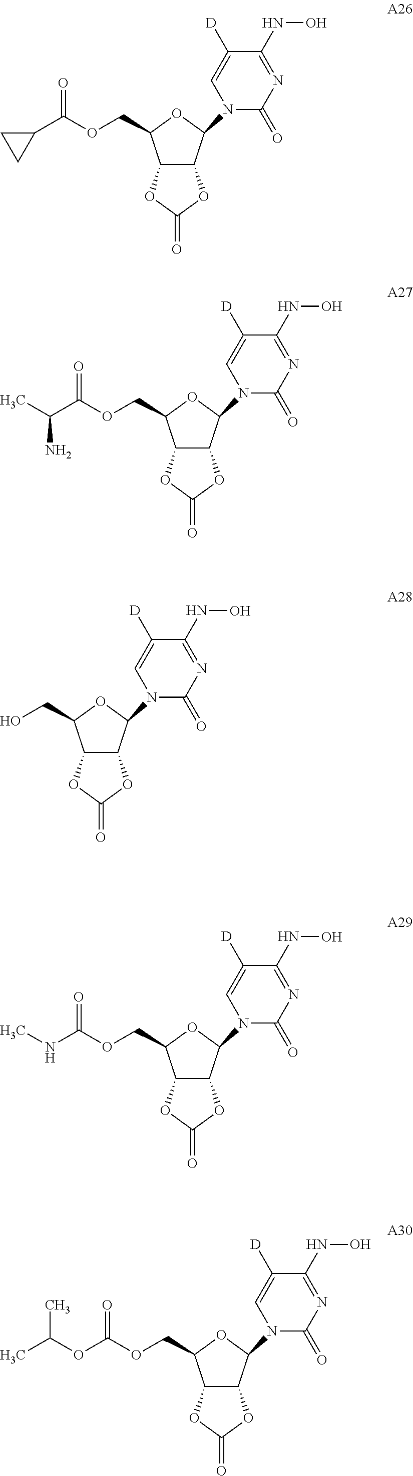 [SMILES: [2H]c1cn([C@@H]2O[C@H](COC(=O)C3CC3)[C@H]3OC(=O)O[C@H]32)c(=O)nc1NO ; [2H]c1cn([C@@H]2O[C@H](COC(=O)[C@H](C)N)[C@H]3OC(=O)O[C@H]32)c(=O)nc1NO ; [2H]c1cn([C@@H]2O[C@H](CO)[C@H]3OC(=O)O[C@H]32)c(=O)nc1NO ; [2H]c1cn([C@@H]2O[C@H](COC(=O)NC)[C@H]3OC(=O)O[C@H]32)c(=O)nc1NO ; [2H]c1cn([C@@H]2O[C@H](COC(=O)OC(C)C)[C@H]3OC(=O)O[C@H]32)c(=O)nc1NO]

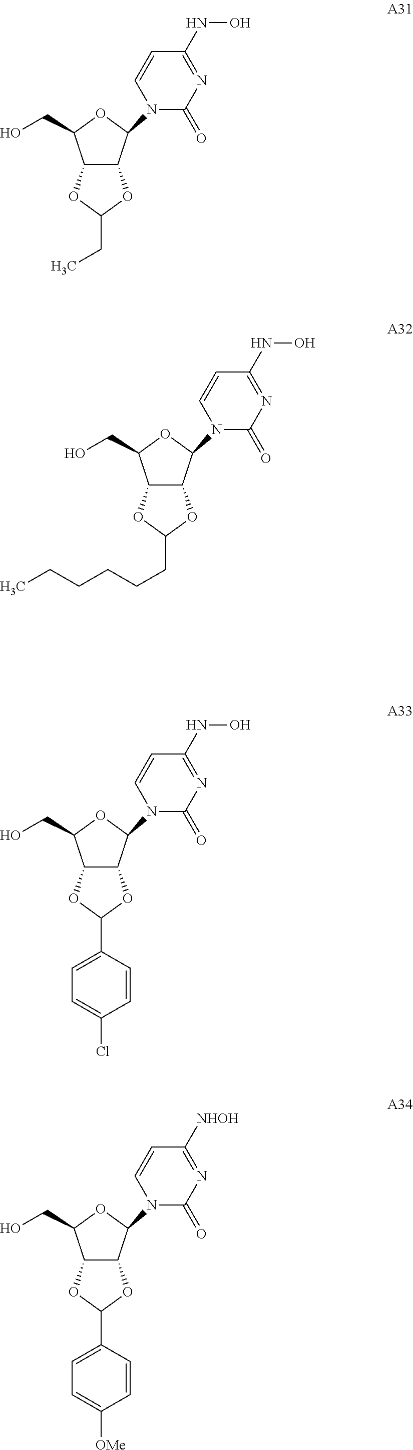

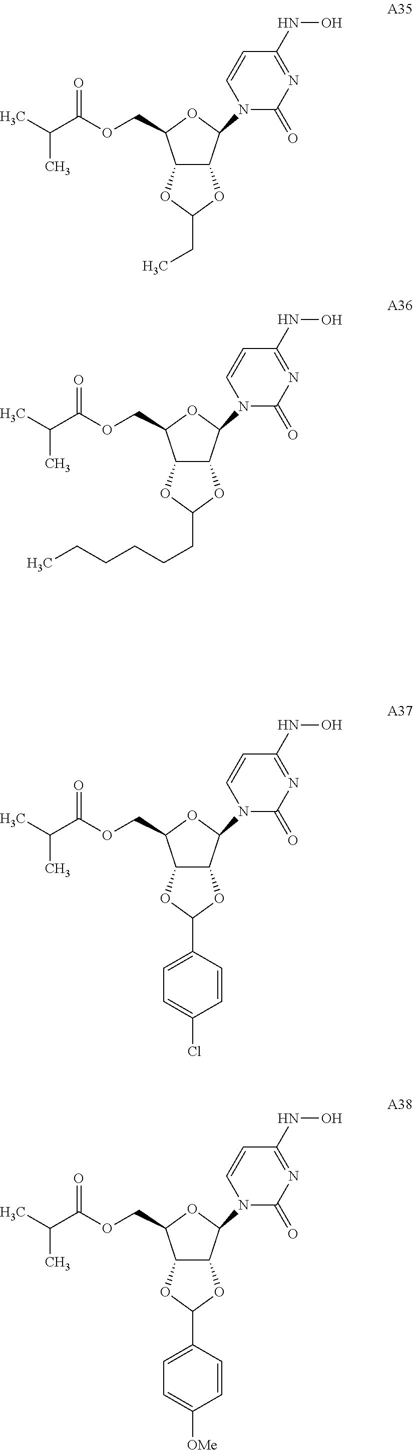

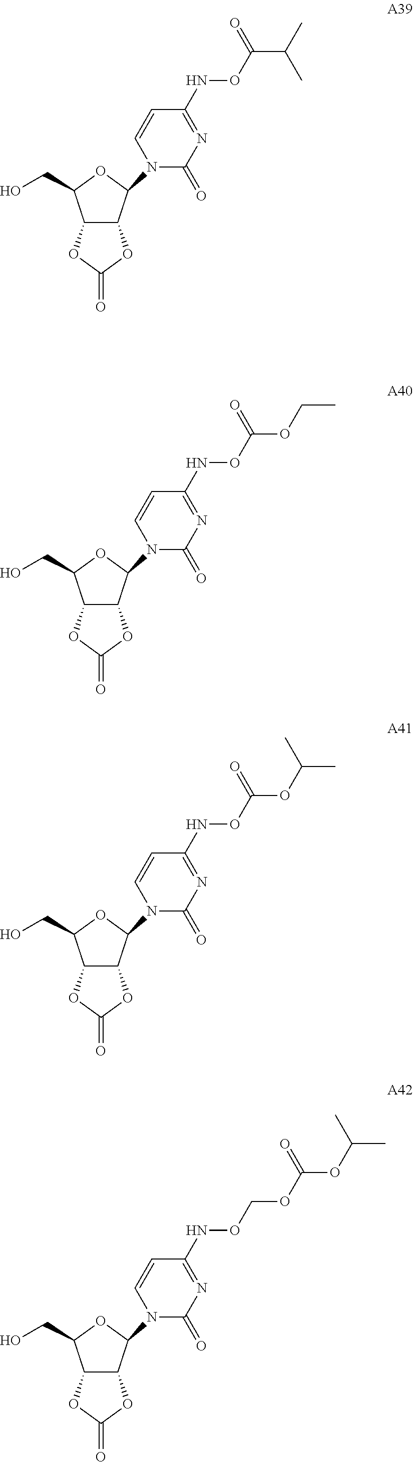 [SMILES: CC(C)C(=O)ONc1ccn([C@@H]2O[C@H](CO)[C@H]3OC(=O)O[C@H]32)c(=O)n1 ; CCOC(=O)ONc1ccn([C@@H]2O[C@H](CO)[C@H]3OC(=O)O[C@H]32)c(=O)n1 ; CC(C)OC(=O)ONc1ccn([C@@H]2O[C@H](CO)[C@H]3OC(=O)O[C@H]32)c(=O)n1 ; CC(C)OC(=O)OCONc1ccn([C@@H]2O[C@H](CO)[C@H]3OC(=O)O[C@H]32)c(=O)n1]

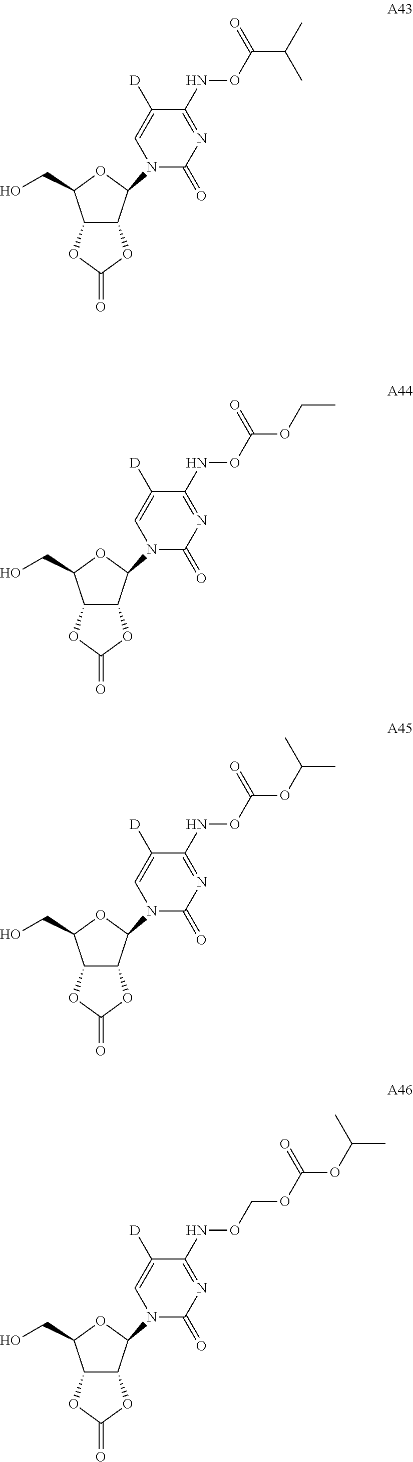 [SMILES: [2H]c1cn([C@@H]2O[C@H](CO)[C@H]3OC(=O)O[C@H]32)c(=O)nc1NOC(=O)C(C)C ; [2H]c1cn([C@@H]2O[C@H](CO)[C@H]3OC(=O)O[C@H]32)c(=O)nc1NOC(=O)OCC ; [2H]c1cn([C@@H]2O[C@H](CO)[C@H]3OC(=O)O[C@H]32)c(=O)nc1NOC(=O)OC(C)C ; [2H]c1cn([C@@H]2O[C@H](CO)[C@H]3OC(=O)O[C@H]32)c(=O)nc1NOCOC(=O)OC(C)C]

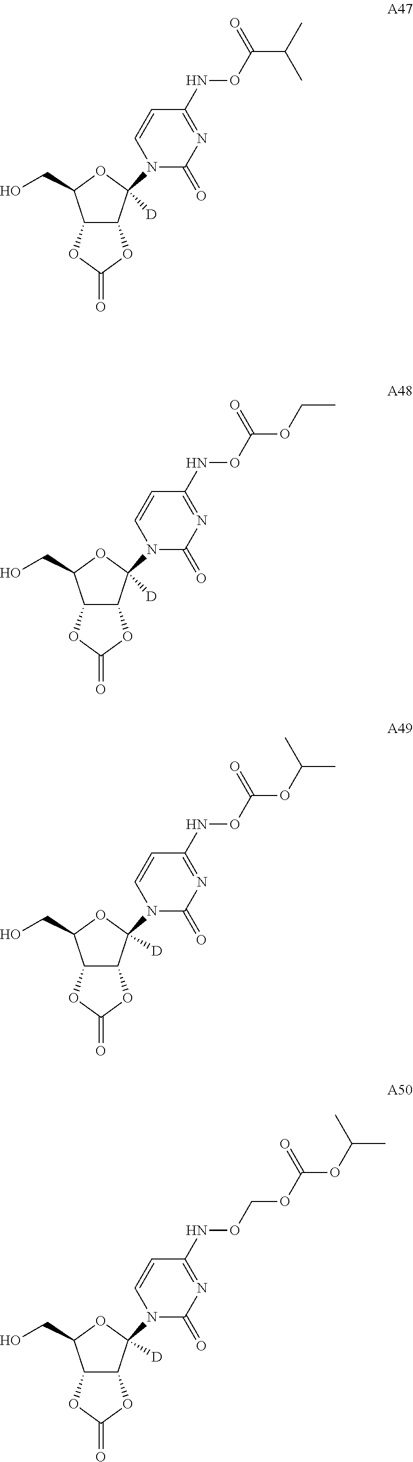 [SMILES: [2H][C@@]1(n2ccc(NOC(=O)C(C)C)nc2=O)O[C@H](CO)[C@H]2OC(=O)O[C@H]21 ; [2H][C@@]1(n2ccc(NOC(=O)OCC)nc2=O)O[C@H](CO)[C@H]2OC(=O)O[C@H]21 ; [2H][C@@]1(n2ccc(NOC(=O)OC(C)C)nc2=O)O[C@H](CO)[C@H]2OC(=O)O[C@H]21 ; [2H][C@@]1(n2ccc(NOCOC(=O)OC(C)C)nc2=O)O[C@H](CO)[C@H]2OC(=O)O[C@H]21]

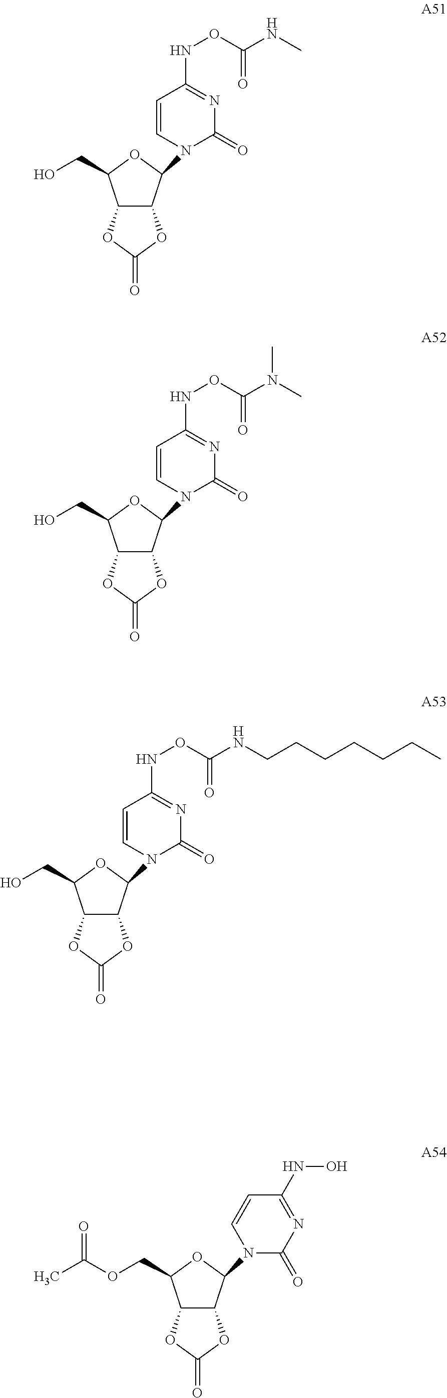

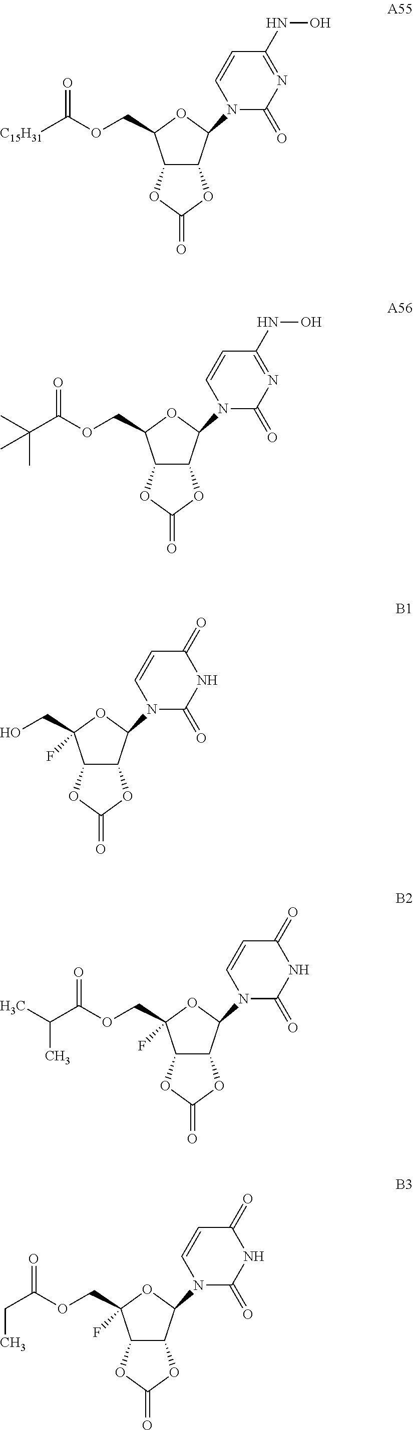

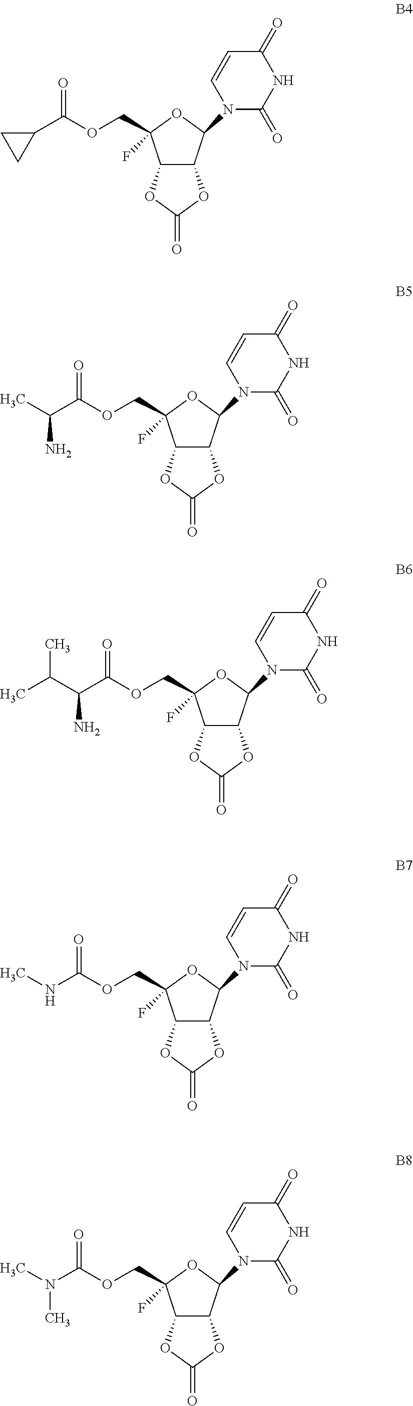 [SMILES: O=C1O[C@H]2[C@H](n3ccc(=O)[nH]c3=O)O[C@](F)(COC(=O)C3CC3)[C@H]2O1 ; C[C@H](N)C(=O)OC[C@@]1(F)O[C@@H](n2ccc(=O)[nH]c2=O)[C@@H]2OC(=O)O[C@@H]21 ; CC(C)[C@H](N)C(=O)OC[C@@]1(F)O[C@@H](n2ccc(=O)[nH]c2=O)[C@@H]2OC(=O)O[C@@H]21 ; CNC(=O)OC[C@@]1(F)O[C@@H](n2ccc(=O)[nH]c2=O)[C@@H]2OC(=O)O[C@@H]21 ; CN(C)C(=O)OC[C@@]1(F)O[C@@H](n2ccc(=O)[nH]c2=O)[C@@H]2OC(=O)O[C@@H]21]

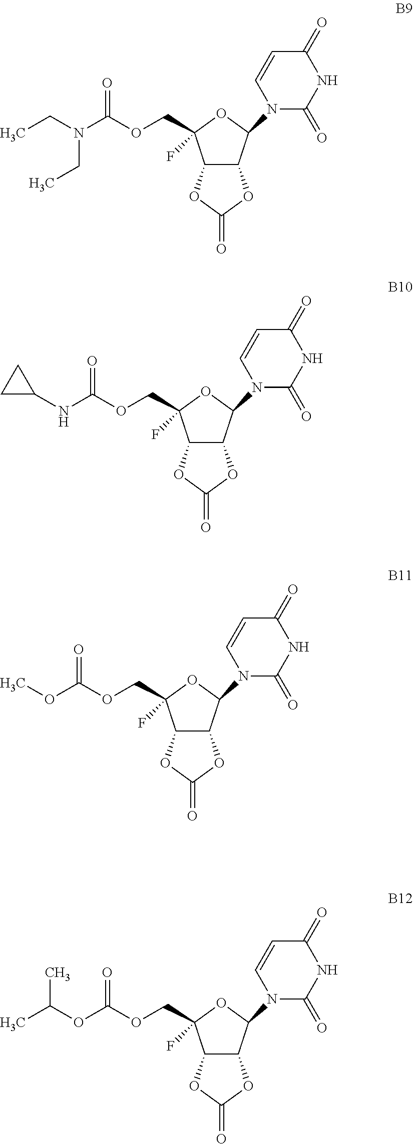 [SMILES: CCN(CC)C(=O)OC[C@@]1(F)O[C@@H](n2ccc(=O)[nH]c2=O)[C@@H]2OC(=O)O[C@@H]21 ; O=C(NC1CC1)OC[C@@]1(F)O[C@@H](n2ccc(=O)[nH]c2=O)[C@@H]2OC(=O)O[C@@H]21 ; COC(=O)OC[C@@]1(F)O[C@@H](n2ccc(=O)[nH]c2=O)[C@@H]2OC(=O)O[C@@H]21 ; CC(C)OC(=O)OC[C@@]1(F)O[C@@H](n2ccc(=O)[nH]c2=O)[C@@H]2OC(=O)O[C@@H]21]

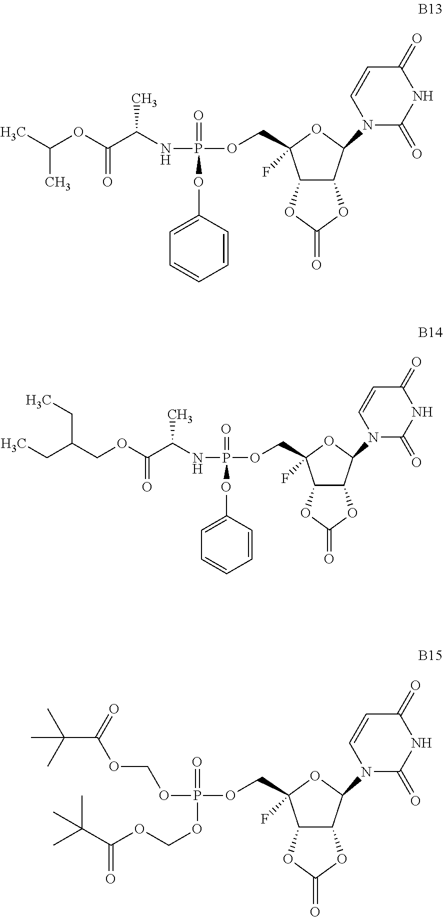 [SMILES: CC(C)OC(=O)[C@H](C)N[P@](=O)(OC[C@@]1(F)O[C@@H](n2ccc(=O)[nH]c2=O)[C@@H]2OC(=O)O[C@@H]21)Oc1ccccc1 ; CCC(CC)COC(=O)[C@H](C)N[P@](=O)(OC[C@@]1(F)O[C@@H](n2ccc(=O)[nH]c2=O)[C@@H]2OC(=O)O[C@@H]21)Oc1ccccc1 ; CC(C)(C)C(=O)OCOP(=O)(OCOC(=O)C(C)(C)C)OC[C@@]1(F)O[C@@H](n2ccc(=O)[nH]c2=O)[C@@H]2OC(=O)O[C@@H]21]

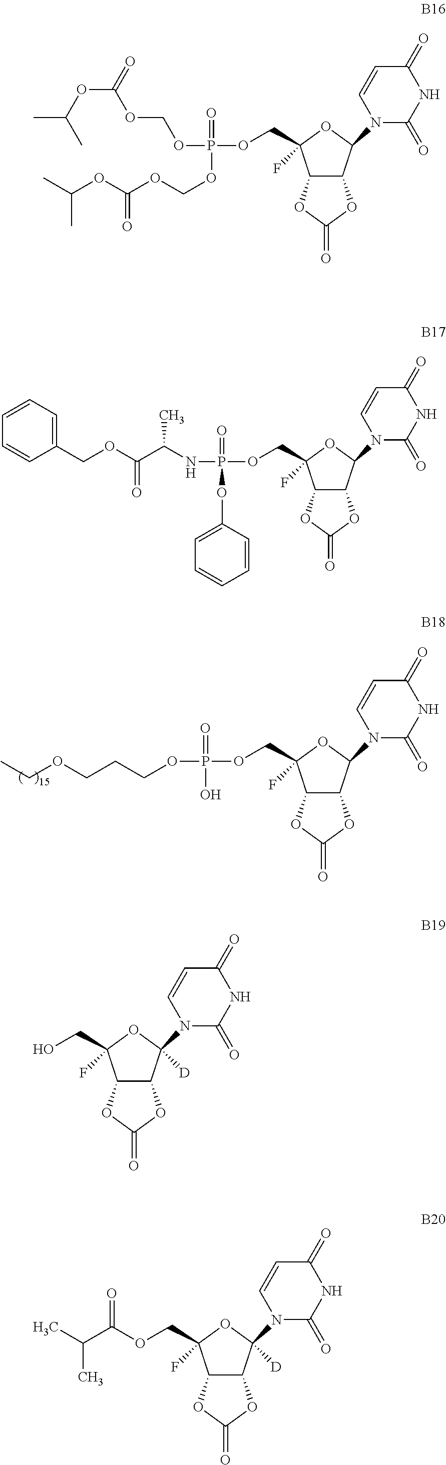

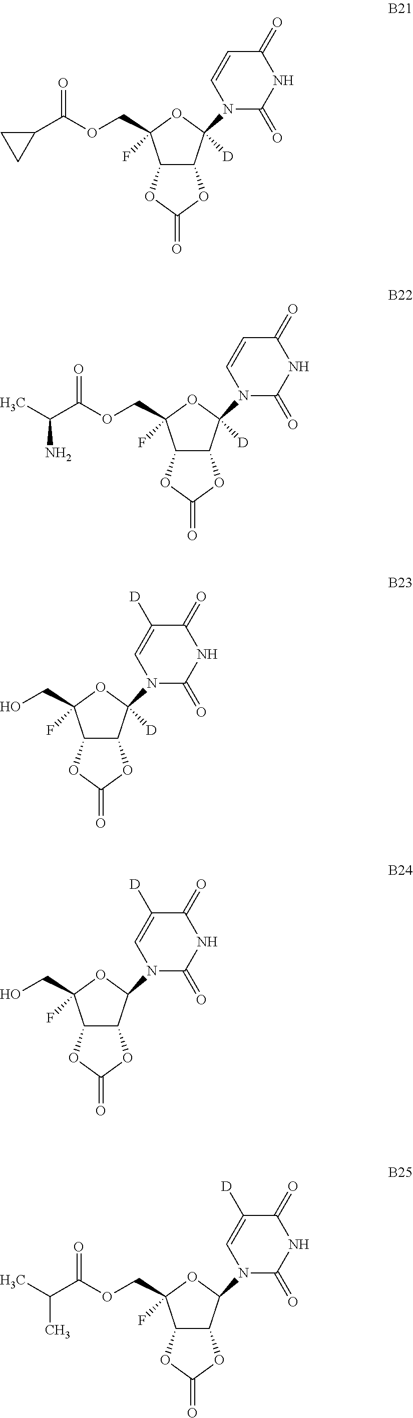 [SMILES: [2H][C@@]1(n2ccc(=O)[nH]c2=O)O[C@](F)(COC(=O)C2CC2)[C@H]2OC(=O)O[C@H]21 ; [2H][C@@]1(n2ccc(=O)[nH]c2=O)O[C@](F)(COC(=O)[C@H](C)N)[C@H]2OC(=O)O[C@H]21 ; [2H]c1cn([C@]2([2H])O[C@](F)(CO)[C@H]3OC(=O)O[C@H]32)c(=O)[nH]c1=O ; [2H]c1cn([C@@H]2O[C@](F)(CO)[C@H]3OC(=O)O[C@@H]23)c(=O)[nH]c1=O ; [2H]c1cn([C@@H]2O[C@](F)(COC(=O)C(C)C)[C@H]3OC(=O)O[C@@H]23)c(=O)[nH]c1=O]

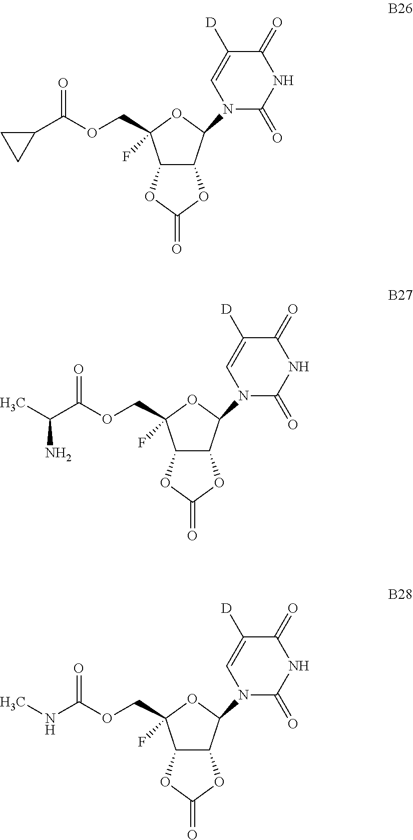 [SMILES: [2H]c1cn([C@@H]2O[C@](F)(COC(=O)C3CC3)[C@H]3OC(=O)O[C@@H]23)c(=O)[nH]c1=O ; [2H]c1cn([C@@H]2O[C@](F)(COC(=O)[C@H](C)N)[C@H]3OC(=O)O[C@@H]23)c(=O)[nH]c1=O ; [2H]c1cn([C@@H]2O[C@](F)(COC(=O)NC)[C@H]3OC(=O)O[C@@H]23)c(=O)[nH]c1=O]

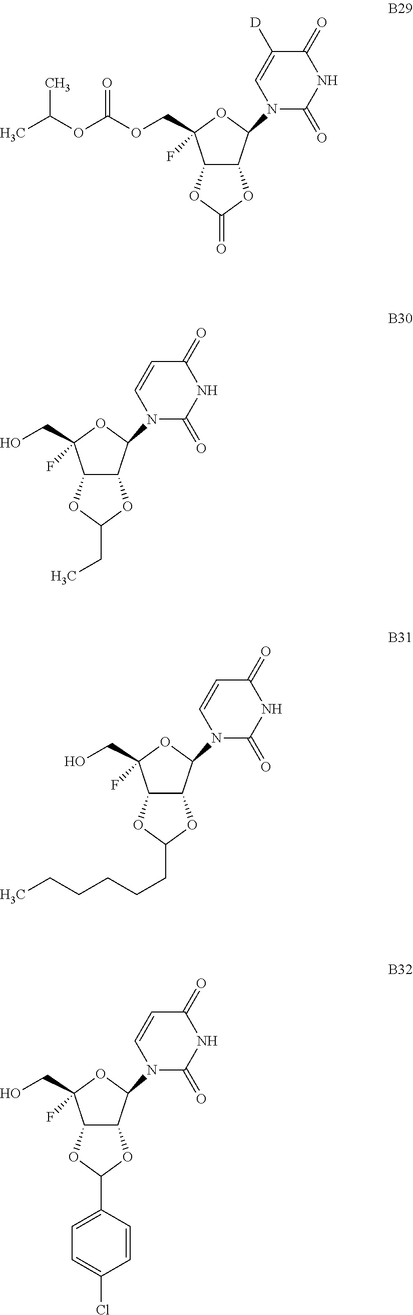

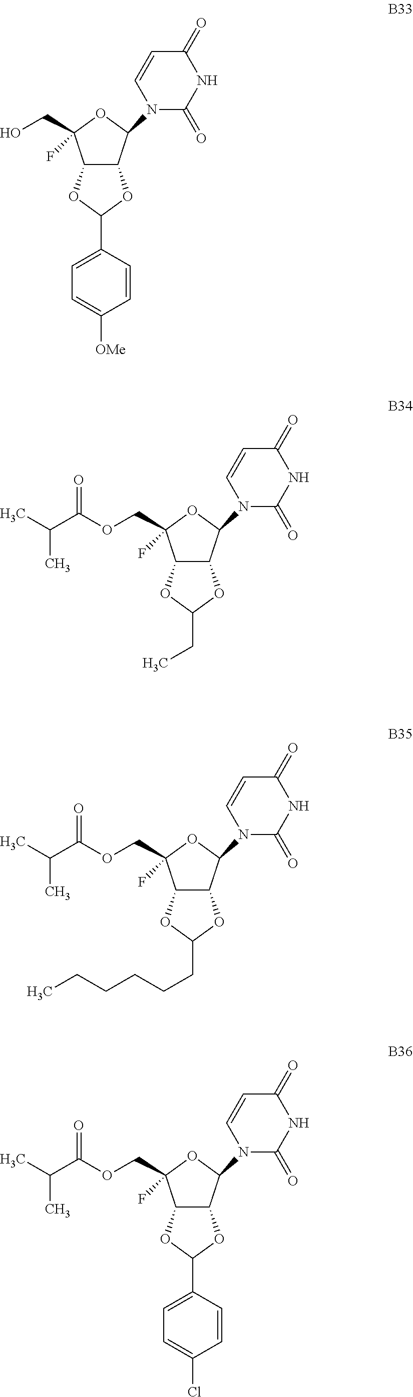

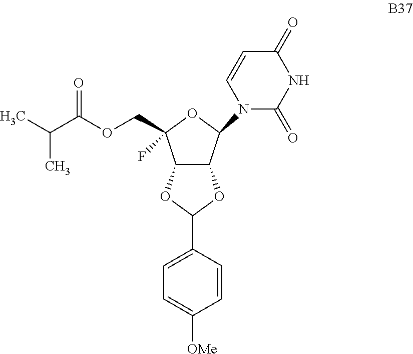

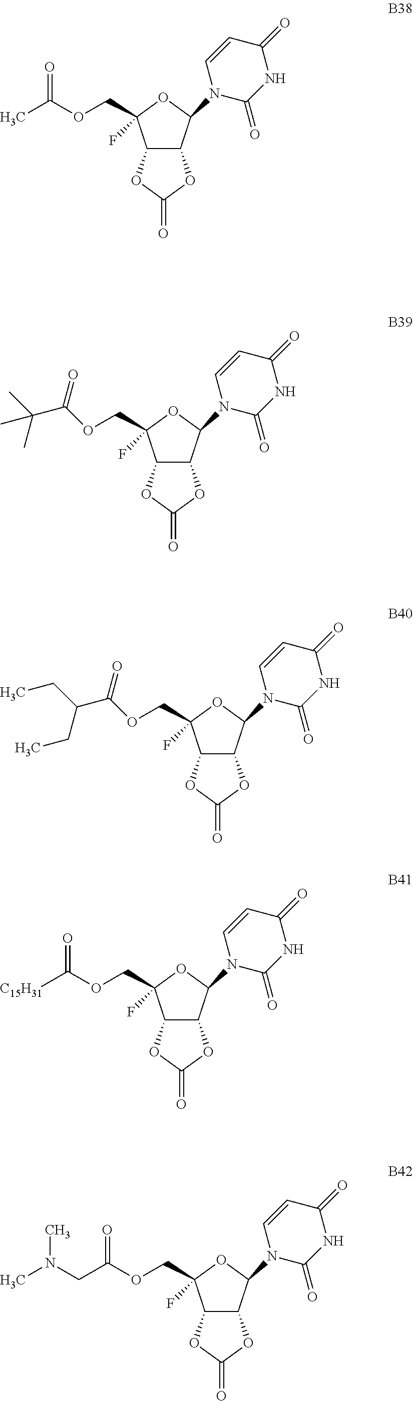

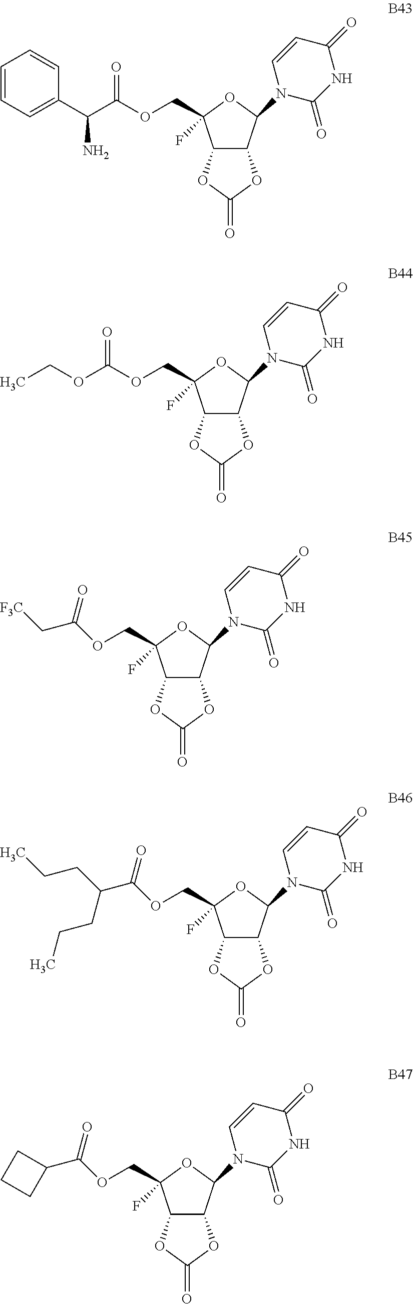 [SMILES: N[C@H](C(=O)OC[C@@]1(F)O[C@@H](n2ccc(=O)[nH]c2=O)[C@@H]2OC(=O)O[C@@H]21)c1ccccc1 ; CCOC(=O)OC[C@@]1(F)O[C@@H](n2ccc(=O)[nH]c2=O)[C@@H]2OC(=O)O[C@@H]21 ; O=C(CC(F)(F)F)OC[C@@]1(F)O[C@@H](n2ccc(=O)[nH]c2=O)[C@@H]2OC(=O)O[C@@H]21 ; CCCC(CCC)C(=O)OC[C@@]1(F)O[C@@H](n2ccc(=O)[nH]c2=O)[C@@H]2OC(=O)O[C@@H]21 ; O=C1O[C@H]2[C@H](n3ccc(=O)[nH]c3=O)O[C@](F)(COC(=O)C3CCC3)[C@H]2O1]

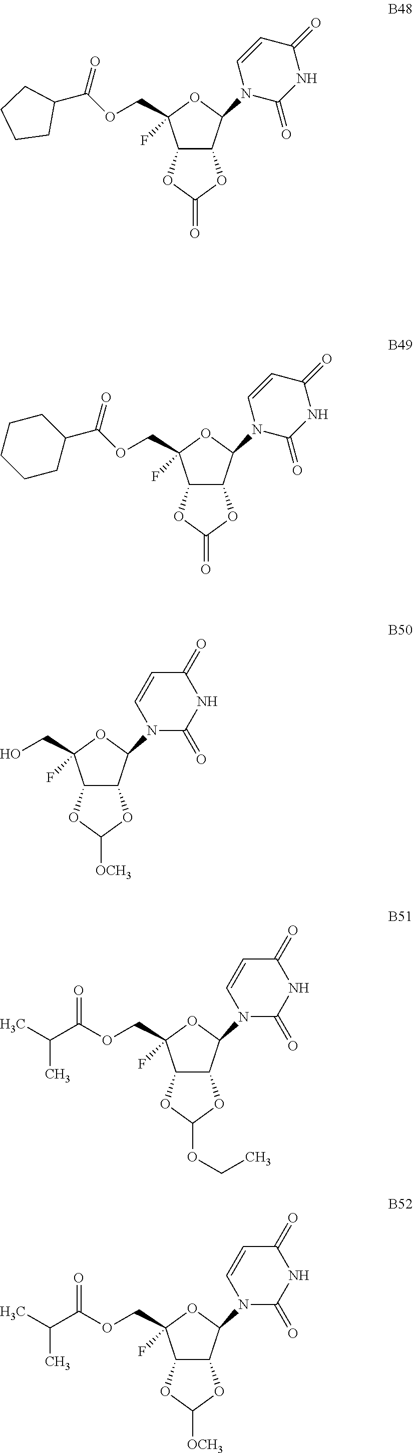

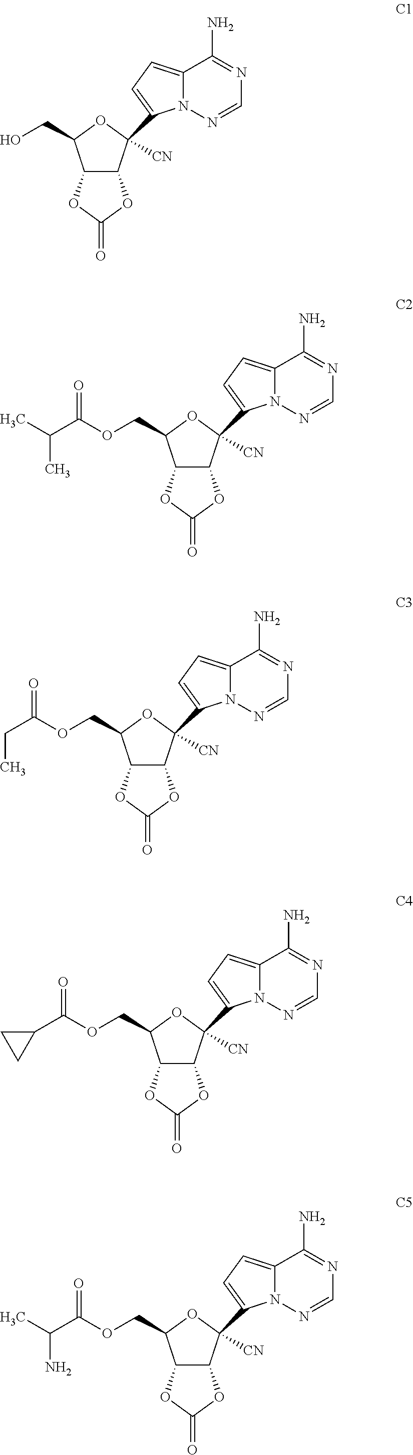 [SMILES: N#C[C@@]1(c2ccc3c(N)ncnn23)O[C@H](CO)[C@H]2OC(=O)O[C@H]21 ; CC(C)C(=O)OC[C@H]1O[C@@](C#N)(c2ccc3c(N)ncnn23)[C@@H]2OC(=O)O[C@@H]21 ; CCC(=O)OC[C@H]1O[C@@](C#N)(c2ccc3c(N)ncnn23)[C@@H]2OC(=O)O[C@@H]21 ; N#C[C@@]1(c2ccc3c(N)ncnn23)O[C@H](COC(=O)C2CC2)[C@H]2OC(=O)O[C@H]21 ; CC(N)C(=O)OC[C@H]1O[C@@](C#N)(c2ccc3c(N)ncnn23)[C@@H]2OC(=O)O[C@@H]21]

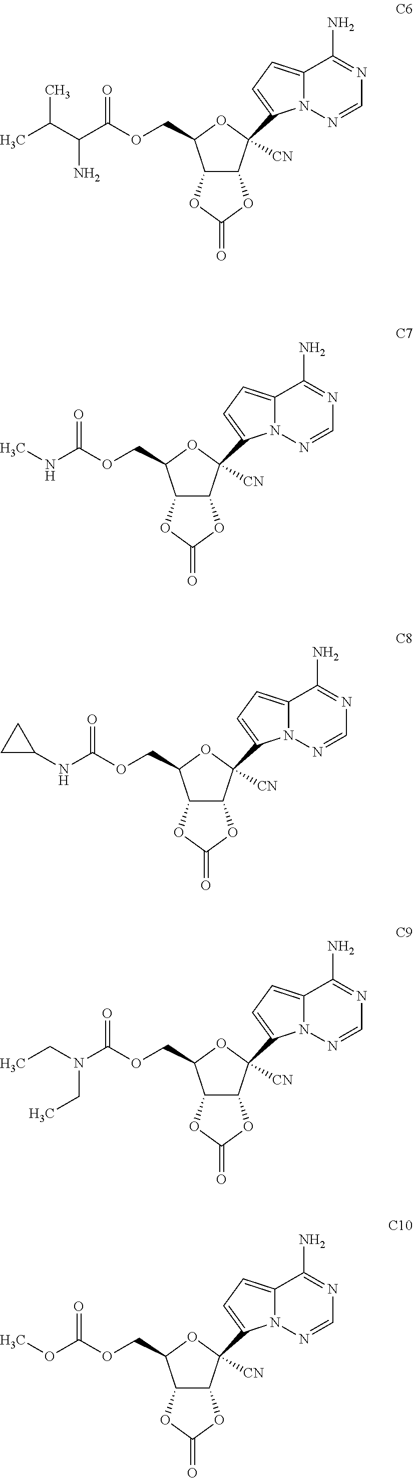 [SMILES: CC(C)C(N)C(=O)OC[C@H]1O[C@@](C#N)(c2ccc3c(N)ncnn23)[C@@H]2OC(=O)O[C@@H]21 ; CNC(=O)OC[C@H]1O[C@@](C#N)(c2ccc3c(N)ncnn23)[C@@H]2OC(=O)O[C@@H]21 ; N#C[C@@]1(c2ccc3c(N)ncnn23)O[C@H](COC(=O)NC2CC2)[C@H]2OC(=O)O[C@H]21 ; CCN(CC)C(=O)OC[C@H]1O[C@@](C#N)(c2ccc3c(N)ncnn23)[C@@H]2OC(=O)O[C@@H]21 ; COC(=O)OC[C@H]1O[C@@](C#N)(c2ccc3c(N)ncnn23)[C@@H]2OC(=O)O[C@@H]21]

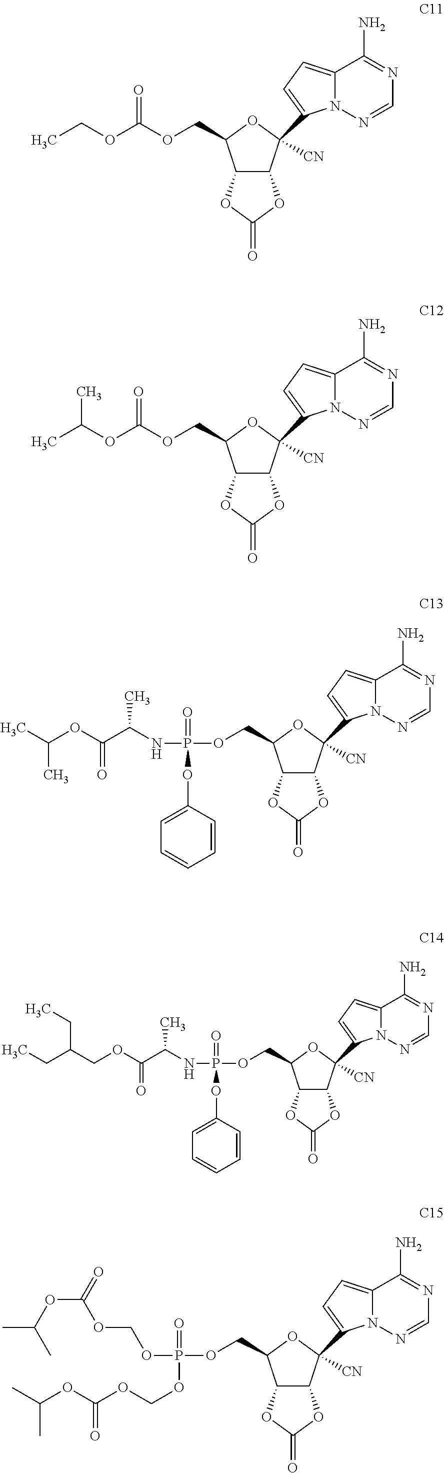 [SMILES: CCOC(=O)OC[C@H]1O[C@@](C#N)(c2ccc3c(N)ncnn23)[C@@H]2OC(=O)O[C@@H]21 ; CC(C)OC(=O)OC[C@H]1O[C@@](C#N)(c2ccc3c(N)ncnn23)[C@@H]2OC(=O)O[C@@H]21 ; CC(C)OC(=O)[C@H](C)N[P@](=O)(OC[C@H]1O[C@@](C#N)(c2ccc3c(N)ncnn23)[C@@H]2OC(=O)O[C@@H]21)Oc1ccccc1 ; CCC(CC)COC(=O)[C@H](C)N[P@](=O)(OC[C@H]1O[C@@](C#N)(c2ccc3c(N)ncnn23)[C@@H]2OC(=O)O[C@@H]21)Oc1ccccc1 ; CC(C)OC(=O)OCOP(=O)(OCOC(=O)OC(C)C)OC[C@H]1O[C@@](C#N)(c2ccc3c(N)ncnn23)[C@@H]2OC(=O)O[C@@H]21]

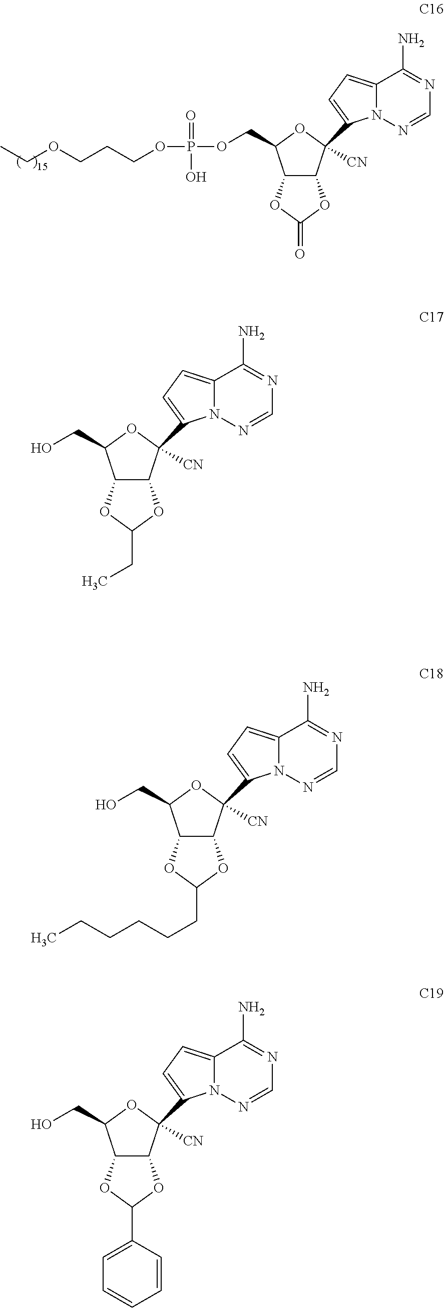

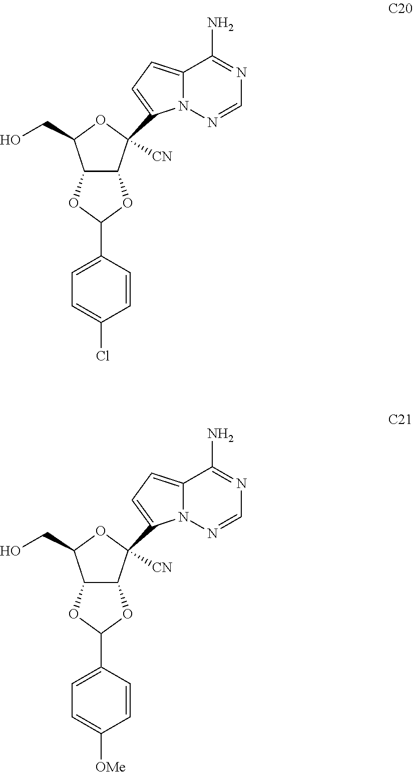

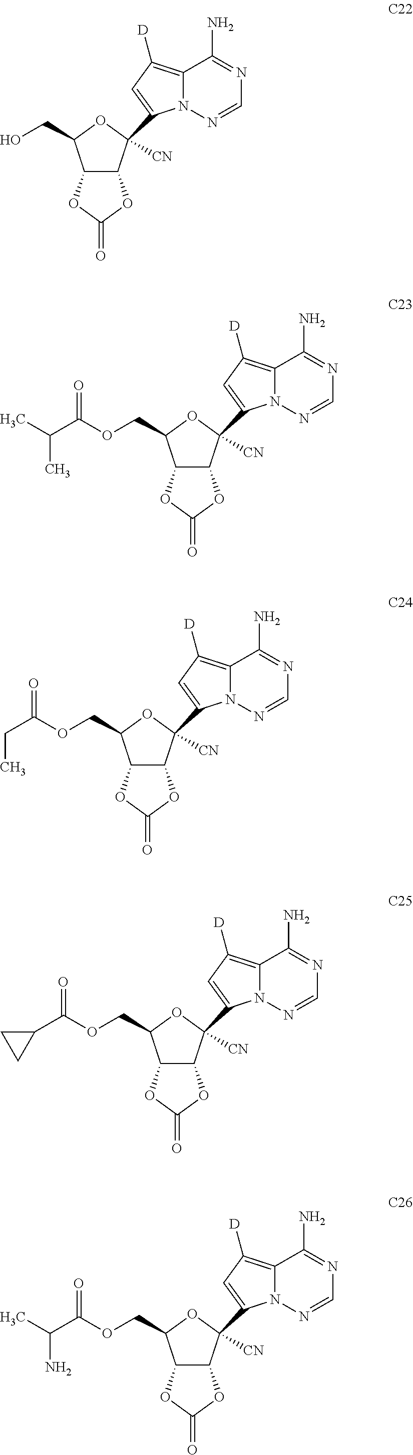 [SMILES: [2H]c1cc([C@]2(C#N)O[C@H](CO)[C@H]3OC(=O)O[C@H]32)n2ncnc(N)c12 ; [2H]c1cc([C@]2(C#N)O[C@H](COC(=O)C(C)C)[C@H]3OC(=O)O[C@H]32)n2ncnc(N)c12 ; [2H]c1cc([C@]2(C#N)O[C@H](COC(=O)CC)[C@H]3OC(=O)O[C@H]32)n2ncnc(N)c12 ; [2H]c1cc([C@]2(C#N)O[C@H](COC(=O)C3CC3)[C@H]3OC(=O)O[C@H]32)n2ncnc(N)c12 ; [2H]c1cc([C@]2(C#N)O[C@H](COC(=O)C(C)N)[C@H]3OC(=O)O[C@H]32)n2ncnc(N)c12]

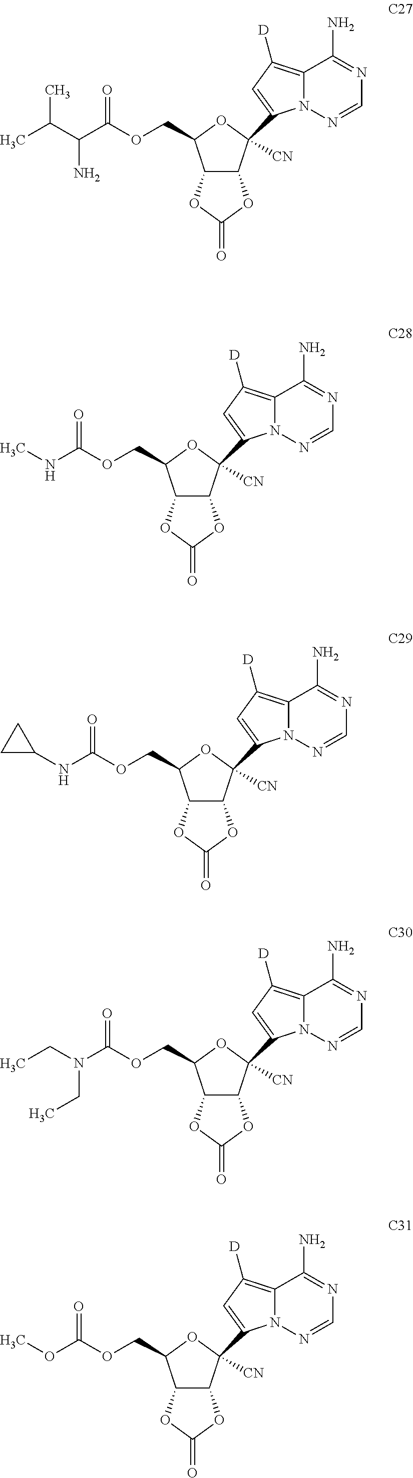 [SMILES: [2H]c1cc([C@]2(C#N)O[C@H](COC(=O)C(N)C(C)C)[C@H]3OC(=O)O[C@H]32)n2ncnc(N)c12 ; [2H]c1cc([C@]2(C#N)O[C@H](COC(=O)NC)[C@H]3OC(=O)O[C@H]32)n2ncnc(N)c12 ; [2H]c1cc([C@]2(C#N)O[C@H](COC(=O)NC3CC3)[C@H]3OC(=O)O[C@H]32)n2ncnc(N)c12 ; [2H]c1cc([C@]2(C#N)O[C@H](COC(=O)N(CC)CC)[C@H]3OC(=O)O[C@H]32)n2ncnc(N)c12 ; [2H]c1cc([C@]2(C#N)O[C@H](COC(=O)OC)[C@H]3OC(=O)O[C@H]32)n2ncnc(N)c12]

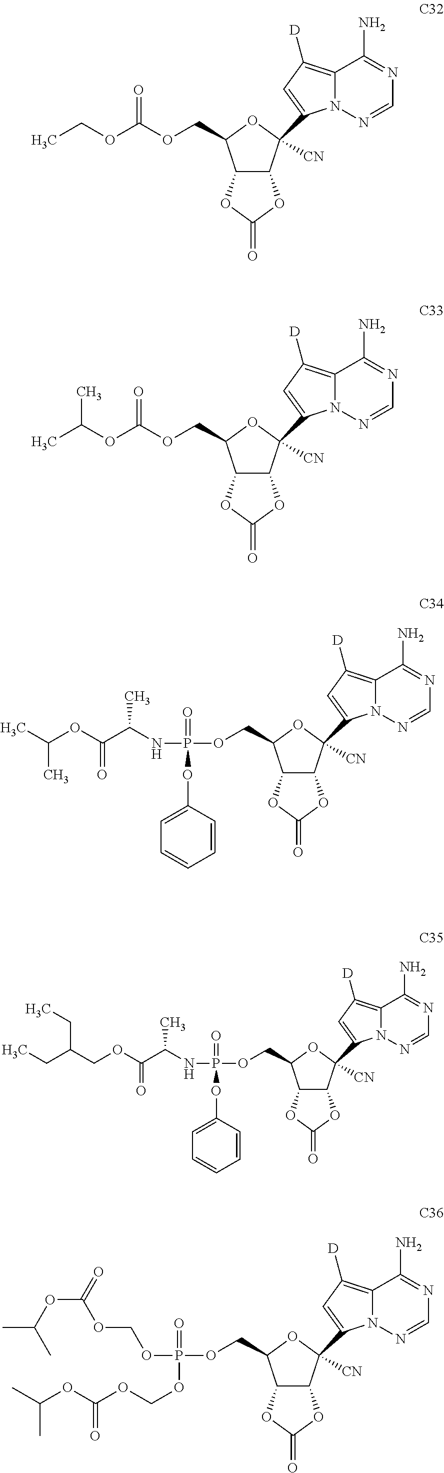 [SMILES: [2H]c1cc([C@]2(C#N)O[C@H](COC(=O)OCC)[C@H]3OC(=O)O[C@H]32)n2ncnc(N)c12 ; [2H]c1cc([C@]2(C#N)O[C@H](COC(=O)OC(C)C)[C@H]3OC(=O)O[C@H]32)n2ncnc(N)c12 ; [2H]c1cc([C@]2(C#N)O[C@H](CO[P@@](=O)(N[C@@H](C)C(=O)OC(C)C)Oc3ccccc3)[C@H]3OC(=O)O[C@H]32)n2ncnc(N)c12 ; [2H]c1cc([C@]2(C#N)O[C@H](CO[P@@](=O)(N[C@@H](C)C(=O)OCC(CC)CC)Oc3ccccc3)[C@H]3OC(=O)O[C@H]32)n2ncnc(N)c12 ; [2H]c1cc([C@]2(C#N)O[C@H](COP(=O)(OCOC(=O)OC(C)C)OCOC(=O)OC(C)C)[C@H]3OC(=O)O[C@H]32)n2ncnc(N)c12]

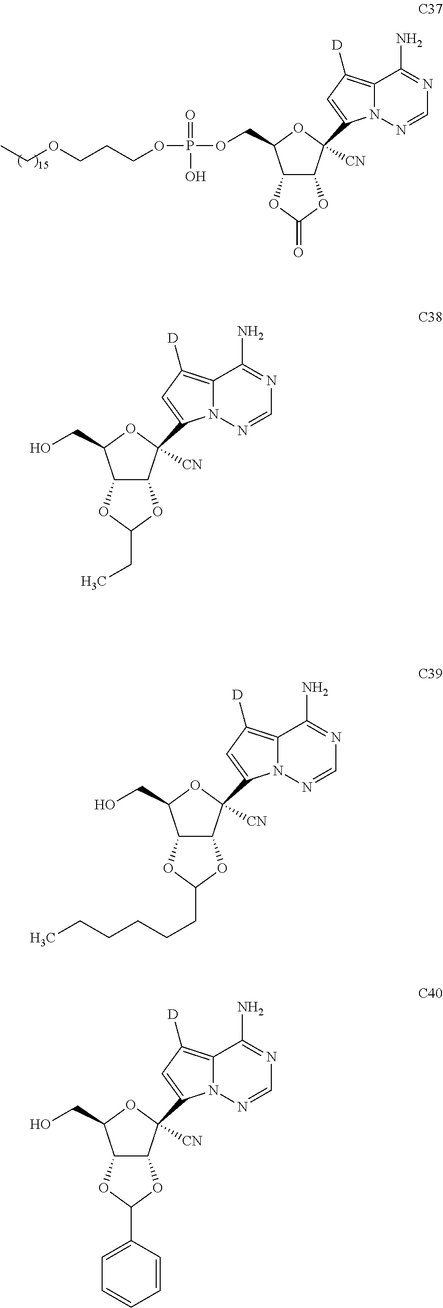

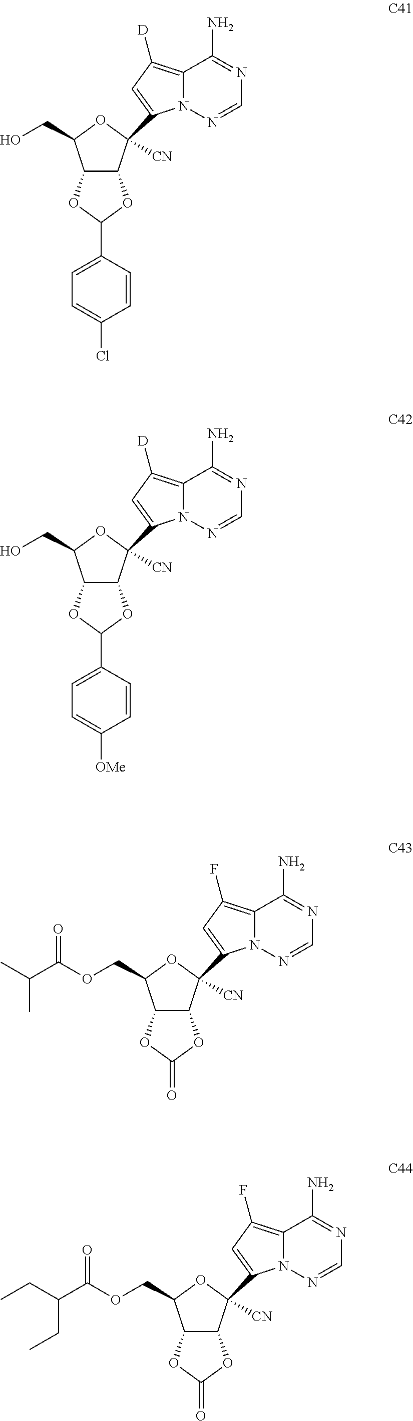

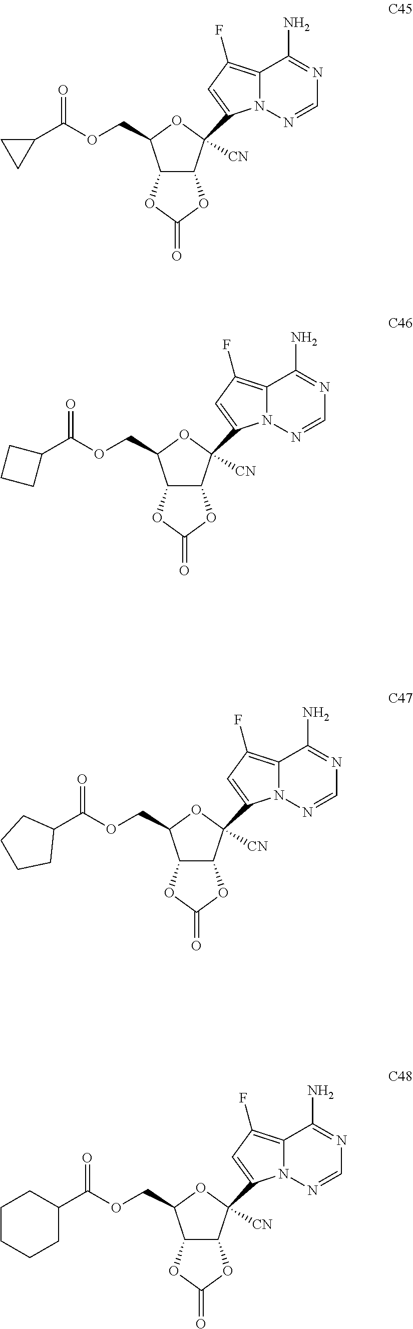 [SMILES: N#C[C@@]1(c2cc(F)c3c(N)ncnn23)O[C@H](COC(=O)C2CC2)[C@H]2OC(=O)O[C@H]21 ; N#C[C@@]1(c2cc(F)c3c(N)ncnn23)O[C@H](COC(=O)C2CCC2)[C@H]2OC(=O)O[C@H]21 ; N#C[C@@]1(c2cc(F)c3c(N)ncnn23)O[C@H](COC(=O)C2CCCC2)[C@H]2OC(=O)O[C@H]21 ; N#C[C@@]1(c2cc(F)c3c(N)ncnn23)O[C@H](COC(=O)C2CCCCC2)[C@H]2OC(=O)O[C@H]21]

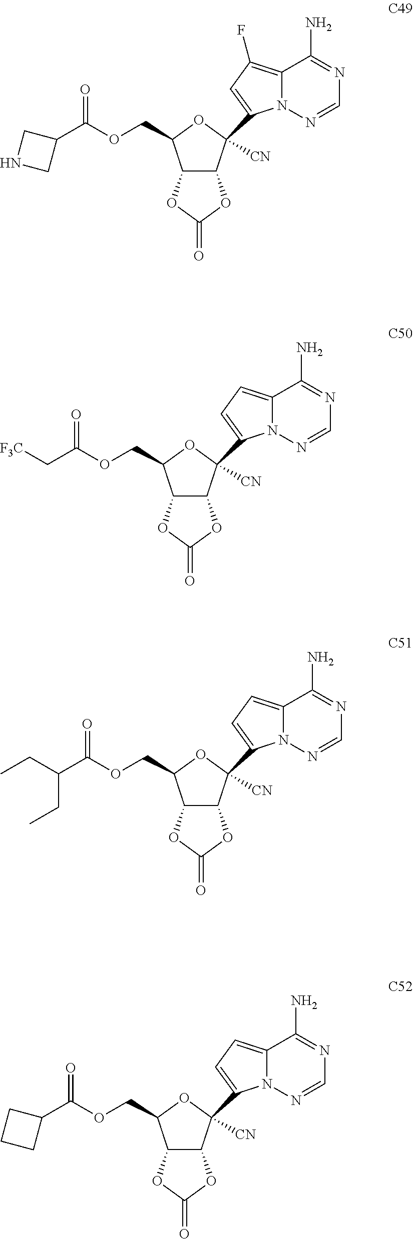 [SMILES: N#C[C@@]1(c2cc(F)c3c(N)ncnn23)O[C@H](COC(=O)C2CNC2)[C@H]2OC(=O)O[C@H]21 ; N#C[C@@]1(c2ccc3c(N)ncnn23)O[C@H](COC(=O)CC(F)(F)F)[C@H]2OC(=O)O[C@H]21 ; CCC(CC)C(=O)OC[C@H]1O[C@@](C#N)(c2ccc3c(N)ncnn23)[C@@H]2OC(=O)O[C@@H]21 ; N#C[C@@]1(c2ccc3c(N)ncnn23)O[C@H](COC(=O)C2CCC2)[C@H]2OC(=O)O[C@H]21]

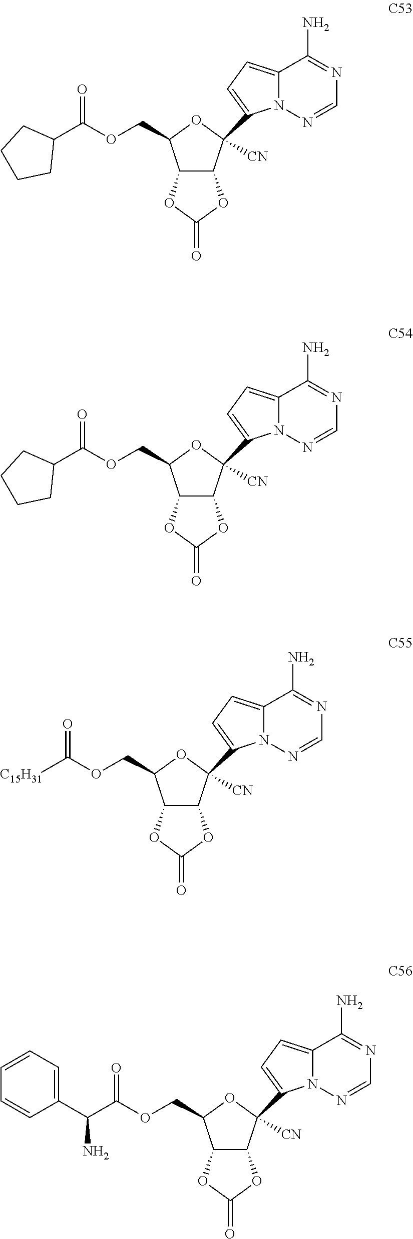

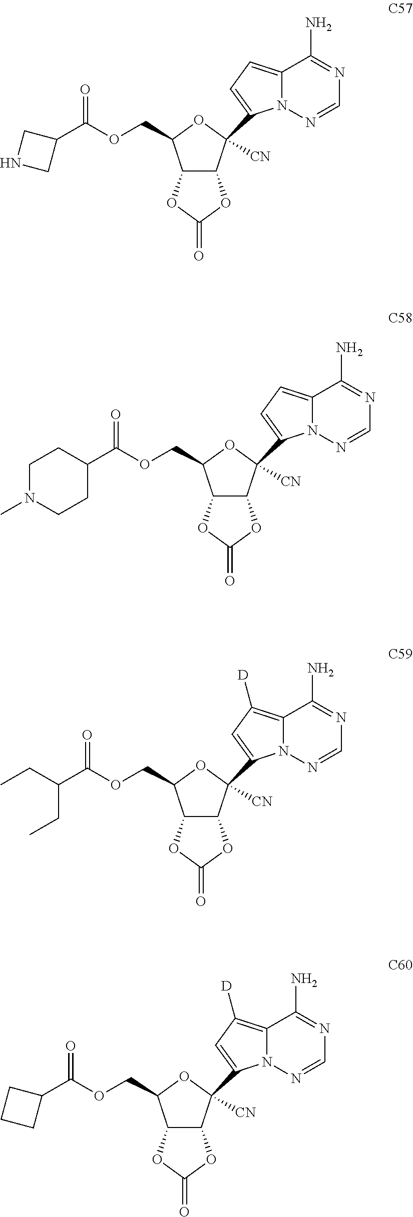 [SMILES: N#C[C@@]1(c2ccc3c(N)ncnn23)O[C@H](COC(=O)C2CNC2)[C@H]2OC(=O)O[C@H]21 ; CN1CCC(C(=O)OC[C@H]2O[C@@](C#N)(c3ccc4c(N)ncnn34)[C@@H]3OC(=O)O[C@@H]32)CC1 ; [2H]c1cc([C@]2(C#N)O[C@H](COC(=O)C(CC)CC)[C@H]3OC(=O)O[C@H]32)n2ncnc(N)c12 ; [2H]c1cc([C@]2(C#N)O[C@H](COC(=O)C3CCC3)[C@H]3OC(=O)O[C@H]32)n2ncnc(N)c12]

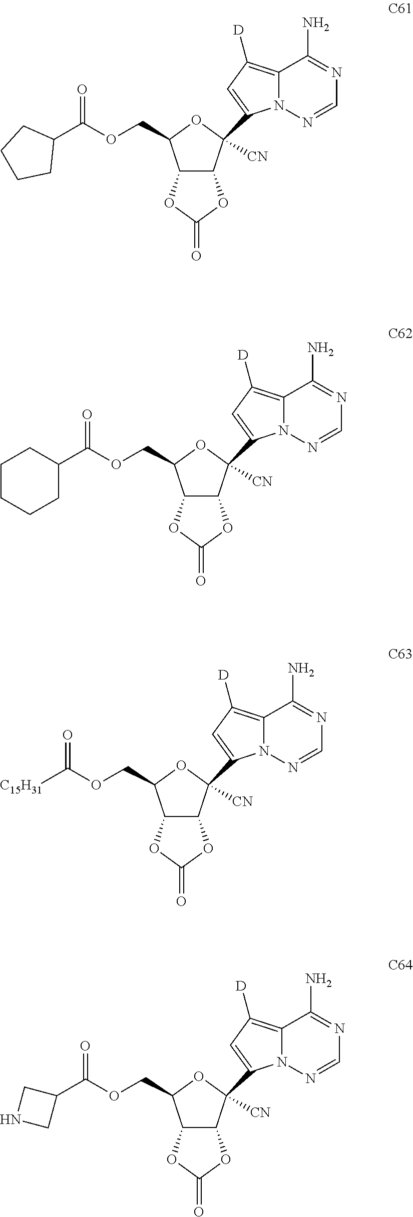

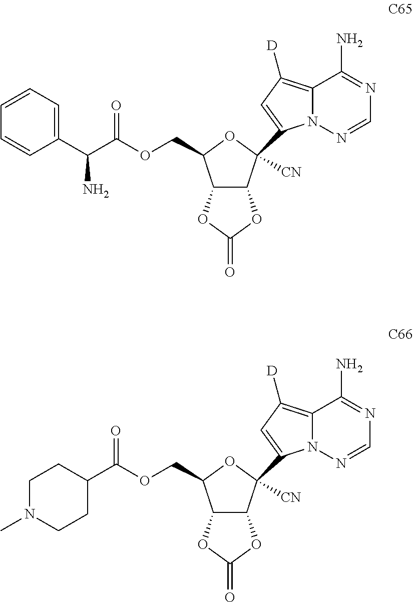 [SMILES: [2H]c1cc([C@]2(C#N)O[C@H](COC(=O)[C@@H](N)c3ccccc3)[C@H]3OC(=O)O[C@H]32)n2ncnc(N)c12 ; [2H]c1cc([C@]2(C#N)O[C@H](COC(=O)C3CCN(C)CC3)[C@H]3OC(=O)O[C@H]32)n2ncnc(N)c12]

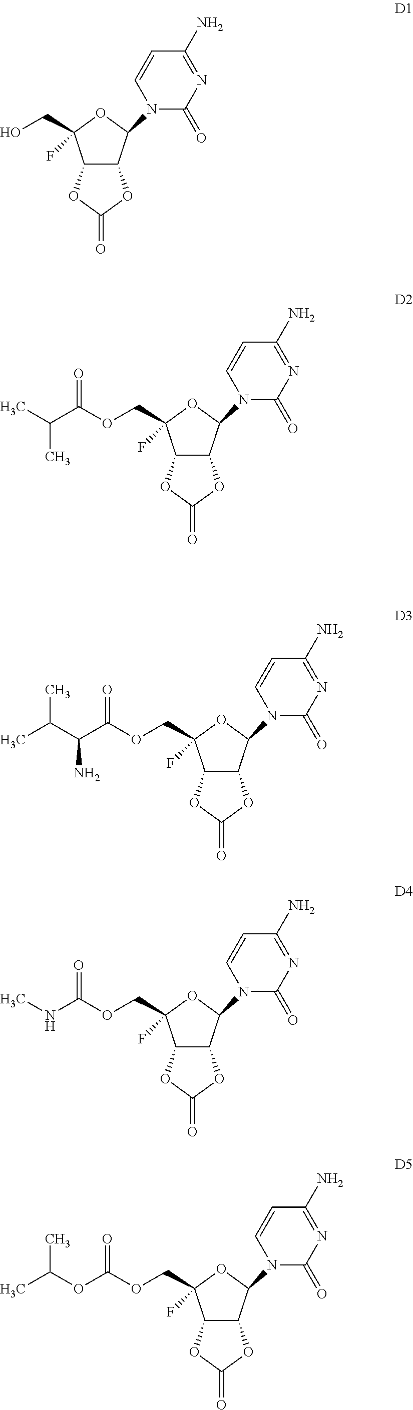 [SMILES: Nc1ccn([C@@H]2O[C@](F)(CO)[C@H]3OC(=O)O[C@@H]23)c(=O)n1 ; CC(C)C(=O)OC[C@@]1(F)O[C@@H](n2ccc(N)nc2=O)[C@@H]2OC(=O)O[C@@H]21 ; CC(C)[C@H](N)C(=O)OC[C@@]1(F)O[C@@H](n2ccc(N)nc2=O)[C@@H]2OC(=O)O[C@@H]21 ; CNC(=O)OC[C@@]1(F)O[C@@H](n2ccc(N)nc2=O)[C@@H]2OC(=O)O[C@@H]21 ; CC(C)OC(=O)OC[C@@]1(F)O[C@@H](n2ccc(N)nc2=O)[C@@H]2OC(=O)O[C@@H]21]

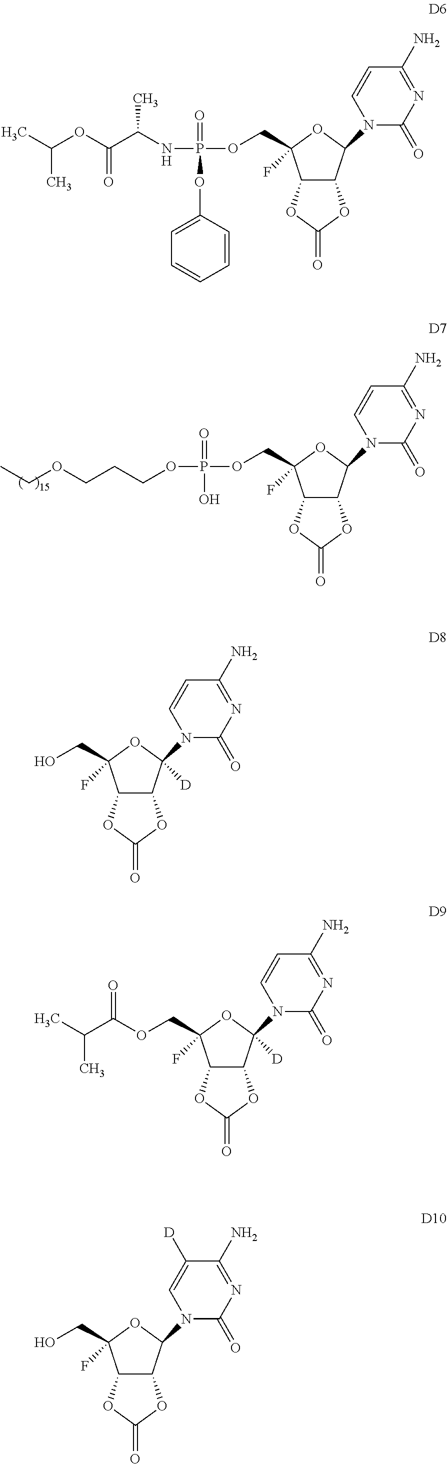

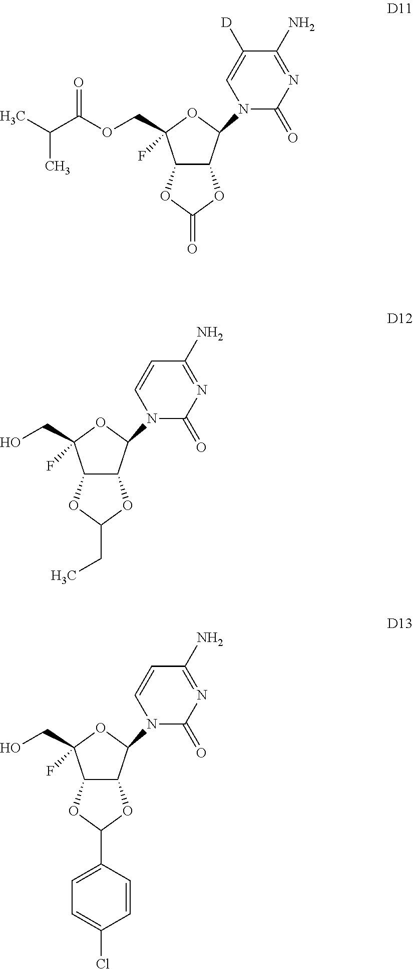

6. A pharmaceutical composition comprising:
(a) one or more selected from the group consisting of the compound and the pharmaceutically acceptable salt thereof according to claim 1, and (b) a pharmaceutically acceptable carrier.

7. A method of inhibiting virus replication or treating or preventing or alleviating a disease caused by a viral infection comprising administering to a subject the compound or the pharmaceutically acceptable salt thereof according claim 1 optionally in the presence of a pharmaceutically acceptable carrier.

8. The method according to claim 7, wherein the virus is one or more selected from the group consisting of:
(1) coronaviruses, such as coronaviruses that infect humans: such as severe acute respiratory syndrome coronavirus (SARS-CoV), 2019 novel coronavirus (SARS-CoV-2), Middle East respiratory syndrome coronavirus (MERS-CoV), Human coronavirus OC43, Human coronavirus 229E, Human coronavirus NL63, Human coronavirus HKU1; and
coronaviruses that infect animals: such as porcine epidemic diarrhea virus (PEDV), feline infectious peritonitis virus (FIFV);
(2) paramyxoviruses: such as paraflu virus, measles virus, respiratory syncytial virus (RSV);
(3) influenza viruses: such as influenza A virus, influenza B virus, influenza C virus, influenza D virus;
(4) flaviviruses: such as hepatitis C virus (HCV), dengue virus (DENY), Zika virus (ZIKV);
(5) filoviruses: such as Marburg virus (MBV), Ebola virus (EBV), Cueva virus;
(6) bunyaviridae viruses: such as Bunyaviviruses, Phleboviruses, Nairoviruses, Hantaviruses;
(7) arenaviruses: such as Lassa fever virus (LASV), Junin virus (JUNV), Machupo virus (MACV);
in particular, the virus is SARS-CoV-2 or an influenza virus.

9. The method according to claim 7, wherein the disease caused by viral infection is one or more selected from the group consisting of:
(D1) common cold, high-risk symptom infection, respiratory tract infection, pneumonia and complications thereof caused by human coronavirus infection;
(D2) porcine epidemic diarrhea caused by porcine epidemic diarrhea virus;
(D3) Feline infectious peritonitis caused by feline coronavirus;
(D4) common cold, high-risk symptom infection, respiratory tract infection, pneumonia and complications thereof caused by human respiratory syncytial virus infection;
(D5) common cold, high-risk symptom infection, respiratory tract infection, pneumonia and complications thereof caused by influenza virus infection;
(D6) chronic hepatitis C and complications thereof caused by hepatitis C virus;
(D7) dengue fever and complications thereof caused by dengue virus;
(D8) infection and complications thereof caused by Zika virus;
(D9) hemorrhagic fever and complications thereof caused by Marburg virus or Ebola virus;
(D10) infection and complications thereof caused by Bunyaviridae viruses;
(D11) infection and complications thereof caused by arenaviruses.

10. The method according to claim 7, wherein the disease caused by viral infection is a disease caused by SARS-CoV-2 infection, in particular one or more selected from the group consisting of respiratory tract infection, pneumonia and complications thereof; or
a disease caused by influenza virus infection; in particular, one or more selected from the group consisting of common cold, high-risk symptom infection, respiratory tract infection, pneumonia and complications thereof.
